(19) United States
(12) Patent Application Publication
(10) Pub. No.: US 2007/0092926 A1
(43) Pub. Date: Apr. 26, 2007

(54) Analysis of protein isoforms using unique tryptic peptides by mass spectrometry and immunochemistry

(76) Inventors: Michail A. Alterman, Bethesda, MD (US); Boris A. Kornilayev, Lawrence, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/581,013

(22) Filed: Oct. 13, 2006

Related U.S. Application Data
(60) Provisional application No. 60/727,171, filed on Oct. 14, 2005.

(51) Int. Cl. C12Q 1/37

(52) U.S. Cl. 435/23

(57) ABSTRACT

A method for qualitatively and quantitatively detecting a protein isoform (p450 isozyme) in a sample using MALDI-TOF mass spectrometry or immunochemistry using a unique proteolytic peptide for the isoform. Relative and absolute quantitation can be performed using calibration curves with P450 isozyme-specific peptide standards.

### CROSS-REFERENCE TO RELATED APPLICATIONS

This application is claims priority to and is based on U.S. Provisional Application Ser. No. 60/727,171 filed on Oct. 14, 2005, which is hereby incorporated herein by reference.

### STATEMENT REGARDING FEDERALLY SPONSORED RESEARCH OR DEVELOPMENT

The present invention was funded in part by the National Institutes of Health, and the government may have certain rights in the invention.

### BACKGROUND OF THE INVENTION

1. Field of the Invention

This present invention relates proteomics. More specifically, it relates to a method for the qualitative and quantitative analysis of protein isoforms/isozymes or any other protein family sharing high degree of homology in complex mixtures representing tissue samples as well as subcellular structures.

2. Description of Related Art

The road to personalized medicine is impossible without knowledge of each patient's unique genetic make-up. However, interrogation of DNA and mRNA information is not enough because only proteins determine real responses on a cellular level. The major objective of personalized medicine is to select individual drug therapies depending upon the correlation of proteomic profiles from diseased tissues with patient response to drug therapy. To a large degree, a person's response is determined by the expression profiles of cytochrome P450 isozymes in a particular tissue. Thus, to establish a correlation between proteomic profiles and drug efficacy, a understanding of the qualitative and quantitative composition of P450 isozymes is desired.

A proteomic case study in personalized medicine is provided by the superfamily of cytochrome P450 enzymes (“CYP”). P450s are the key enzymes responsible for biotransformations of numerous endogenous compounds, i.e. steroids, bile acids, fatty acids, prostaglandins, leukotrienes, and also metabolize a wide range of xenobiotics including drugs, environmental pollutants and alcohols. To date, this superfamily is the largest group of enzymes that share a high degree of similarity in protein sequence. The number of named and sequenced CYPs has already surpassed 6000, what constitutes more than 2% of all known to date proteins. P450s are located in almost every tissue, with the highest concentration in liver and kidney. The human genome encodes at least 57 CYP genes and 58 pseudogenes. The composition of CYP isozymes in a particular tissue determines a human's response to a drug and/or elicits drug-drug interaction or causes changes in a mediator response. Yet, a major gap exists in the knowledge about individual and inter-individual, racial, age and gender differences in CYP isozyme expression on a protein level. The very limited amounts of data available to date were obtained from DNA and mRNA-based experiments. While applicable to proteomics generally, this invention is focused on the development and application of new methods for targeted differential proteomics of CYP isozymes.

Arguably, the largest and most functionally diverse superfamily of cytochrome(s) P450 is of great interest in biomedical research. CYPs have been widely investigated in studies ranging from the molecular biology of CYP isozyme expression to the role of CYP isozymes in clinical pharmacology and toxicology. The P450 field has been constantly reviewed from different perspectives. On the other hand, the field of proteomics, despite its relative youth (the very term “proteomics” was coined by Wilkins 10 years ago, in 1995), is reviewed even more extensively.

Current approaches to the identification and characterization of isozymes, such as the P450s, include: (1) isozyme-selective P450 substrates, (2) isozyme-selective P450 inhibitors, (3) antibody-based CYP isozyme identification, and (4) mRNA-based assessment of CYP isozyme expression. However, each of these approaches suffers from various shortcomings. First, only a minority of known P450 isozymes is fully characterized by substrate specificity, and since they exhibit broad, often overlapping substrate specificity, there is no known substrate or inhibitor that is absolutely specific for an individual P450 isozyme. Second, in many instances there is an absence of any CYP isozyme-selective inhibitor. Third, the high degree of sequence homology among members of the P450 superfamily confounds high specificity of antibody-based analysis, particularly among members of the same subfamily. The application of a quantitative mRNA analysis for the evaluation of P450 isozyme expression, which once looked very promising, is questionable, too. It was shown that in many cases, correlation between protein abundances and mRNA levels for numerous hepatic and extrahepatic proteins is poor. Most importantly, if an unknown or an unexpected P450 isozyme is expressed in the microsomes under investigation, none of these approaches will reveal it.

In recent years, there has been a growing interest in proteomics, using methods involving mass spectrometry (“MS”). Gerber et al. introduced an absolute quantitation method based on the use of peptides synthesized with incorporated stable isotopes with selected reaction monitoring analysis in a tandem ESI MS/MS. See Gerber et al., Absolute quantification of proteins and phosphoproteins from cell lysates by tandem MS, PNAS 100(12): 6940-6945 (2003); see also Gygi et al., U.S. Published Patent No. 2004/0229283 entitled “Absolute quantification of proteins and modified forms thereof by multistage mass spectrometry.” A different twist on the same classical analytical chemistry approach (i.e. use of internal standards for quantitation) was the application of matrix-assisted laser desorption/ionization time of flight mass spectrometry (“MALDI TOF MS”) without introduction of stable isotopes for quantitative analysis by Helmke et al., Simultaneous quantification of human cardiac alpha- and beta-myosin heavy chain proteins by MALDI-TOF mass spectrometry, Anal Chem 76(6): 1683-9 (2004); see also Perryman et al., U.S. Published Patent 2004/0119010 entitled “Quantitative analysis of protein isoforms using matrix-assisted laser desorption/ionization time of flight mass spectrometry.” Yet, the main existing quantitative methods, such as stable isotope labeling by amino acids (“SILAC”) and isotope-coded affinity tag (“ICAT”), could not be applied to quantitation of CYPs. ICAT relies on cysteine containing peptides, and such peptides are conserved among different isozymes, particularly belonging to the same subfamily, not to mention that SILAC as well as ICAT, provide relative not absolute quantitation.

In summary, the drawbacks of current approaches to the identification and quantification protein isoforms (and the cytochrome P450 isozymes in particular) necessitate the need for a development of a comprehensive analytical approach for the determination of CYP isozyme composition.

### BRIEF SUMMARY OF THE INVENTION

This present invention constitutes an analytical method for detecting a protein of interest (e.g., isoform/isozyme) based on the measurement of unique or distinctive proteolytic peptides, such as unique tryptic peptides for the cytochrome P450 isozymes. Mass spectrometry (e.g., MALDI-TOF MS) and immunochemical analysis (e.g., ELISA, Western, dot-blot, or attachment of polyclonal anti-peptide antibodies to Protein A and G magnetic beads) of anti-peptide antibodies developed against the unique proteolytic peptides can be used to detect the protein of interest in a sample both qualitatively and quantitatively.

In one aspect, the present invention overcomes the deficiencies of prior methodologies by taking advantage of MALDI-TOF-MS technology and applying it to proteins and peptides in a way that allows for accurate, quantitative measurement in vivo or in vitro of protein concentrations. Because the unique proteolytic peptides are specific only for the isoform/isozyme that is the protein of interest, the methods can reliably detect and distinguish isoforms/isozymes of a protein family.

In another aspect, the detection methods of the present invention will be useful for drug development, analysis of drug-drug interaction, drug safety assessment and any other area where there is a need to analyze major drug-metabolizing enzymes, such as the cytochromes P450s.

In yet another aspect, the present invention is directed to a method to for detecting a protein of interest contained in a sample. The detection method includes the steps of obtaining a sample; identifying a unique proteolytic peptide derived from the protein of interest by digestion with protease; subjecting the sample to proteolysis using the protease to obtain a mixture of proteolytic peptides; detecting the unique proteolytic peptide in the mixture; wherein the presence or absence of the unique proteolytic peptide in the mixture is indicative of the presence or absence of the protein of interest in the sample.

In another aspect of the invention, the sample may be derived from a cell, a prokaryotic cell, a eukaryotic cell, a mammalian cell, or a human cell. The sample may also be derived from an organ, a human organ, such as the liver. The sample may further be derived from plasma or from serum.

In one embodiment, the detecting step is performed by detecting the unique proteolytic peptide using mass spectrometry, and is preferably matrix-assisted laser desorption/ionization time of flight (MALDI-TOF) mass spectrometry. The standards used to quantitate the concentrations of protein can be produced synthetically. In a variation on the invention, the method may not utilize standards but, rather, may involve determining relative quantities of two proteins by comparing unique aspects of the individual MALDI-TOF profiles, as compared to standard profiles. These proteins may be isoforms/isozymes of each other.

In another embodiment, the detecting step is performed by detecting the unique proteolytic peptide using immunochemistry, and is preferably a fluorescent antibody method, enzyme-linked immunosorbent assay method (ELISA), radioimmunoassay (RIA), or sandwich ELISA method.

In one aspect of the invention, the proteins of interest are isoforms of the same protein, and in another embodiment, these isoforms are isozymes from the cytochrome P450 superfamily.

In one aspect, the detection method is used to detect a protein which is member of the P450 superfamily, and the sample contains multiple isozymes of the P450 superfamily. The detection method is able to reliably and accurately detect and quantify the various P450 isoforms in the sample using unique tryptic peptides associated with each of the isoforms.

In another aspect, the unique proteolytic peptides are produced using a protease which a serine protease, such as trypsin.

In still another aspect, proteins of interest are isozymes of the P450 superfamily and the unique proteolytic peptide is a unique tryptic peptide selected from the group consisting of SEQ ID NO. 1 to 502.

Thus, in one embodiment, the detection method comprises the steps of obtaining a sample containing a cytochrome P450 isozyme; identifying a unique tryptic peptide derived from the cytochrome P450 isozyme; subjecting the sample to proteolysis using trypsin to obtain a mixture of tryptic peptides; detecting the amount of unique tryptic peptide in the mixture using MALDI-TOF MS or immunochemistry; wherein the amount of the unique proteolytic peptide in the mixture is indicative of the amount of cytochrome P450 isozyme in the sample. Exemplary unique tryptic peptides for the P450 cytochrome isozymes to be detected are SEQ ID NO. 1-502.

In still other aspects of the present invention, antibodies that bind to the unique tryptic peptides are provided. In one embodiment, the antibodies bind to an epitope consisting essentially of a unique tryptic peptide derived from a cytochrome P450 isozyme. For example, antibodies that bind to an epitope consisting of the unique tryptic peptides having SEQ ID No. 1-494 will be useful in detecting cytochrome P450 isozymes in a sample. The antibodies are preferably labeled with a reporter group. Exemplary antibodies are those that bind to an epitope that is the CYP2E1 unique tryptic peptide having SEQ ID NO. 88 (FITLVPSNLPHEATR) and antibodies that bind to an epitope that is the CYP1A2 unique tryptic peptide having SEQ ID NO. 13 (YLPNPALQR). The antibodies may exhibit inhibitory properties, for example, inhibition of chloroxazone 6-hydroxylation.

In yet another aspect, the invention includes the approach to predict and calculate presence and/or existence of unique tryptic peptides with respect to the human genome by combination of simulated tryptic digest of proteins of interest followed by comparative analysis of the obtained tryptic peptide sequences with the simulated tryptic digests of all human and non-human proteins in Protein Data Base (SwissProt and NCBI). Examples of calculated P450 isozyme-specific unique tryptic peptides are presented as SEQ ID NO. 1-502.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a general scheme of the proposed invention showing an integrated proteomic analysis flow-chart.

FIG. 2 is a representative MALDI TOF mass spectrum of a tryptic peptide mass fingerprinting (“PMF”) of SDS-PAGE band containing CYP2B1/2B2. Filled circles indicate mass peaks corresponding to common CYP2B1/2B2 tryptic peptides. Open circles correspond to CYP2B1 isozyme-specific tryptic peptides and triangles to CYP2B2 isozyme-specific tryptic peptides. Inset: expanded view showing resolution attained.

FIG. 3 shows the linear dependence between molar ratio of rat CYP2B1 and CYP2B2 isozyme-specific unique tryptic peptides and corresponding monoisotopic peak areas. Each data point is the mean±S.D. of data collected in six experiments.

FIG. 4 is a linearity plot of monoisotopic peak areas of CYP2B1 and CYP2B2 isozyme-specific unique tryptic peptides spiked into BSA (Panel A) and β-LGA (Panel B) tryptic digests. Each data point the mean±S.D. of data collected in six experiments.

FIG. 5 shows the relative quantitation of CYP2B1/2B2 isozymes. Triangles represent monoisotopic peak areas of synthesized CYP2B1 and CYP2B2 isozyme-specific unique tryptic peptides mixtures used to build calibration curve. Open circles represent monoisotopic peak areas of synthesized CYP2B1 and CYP2B2 isozyme-specific unique tryptic peptides spiked into a tryptic digest of a band excised from SDS-PAGE and containing CYP2D2. Calibration curve and experimental samples were extracted with ZipTip C₁₈ and then eluted with MALDI matrix on target. Each data point represents the average±S.D. of data collected in six experiments.

FIG. 6 is a representative tryptic peptide mass fingerprinting MALDI TOF mass spectra of isolated human CYPs. Panel A—CYP1A2 digest (asterisks denote 1A2 tryptic peptides); panel B—CYP1A2 simplified digest without destaining, alkylation and reduction; panel C—CYP2C19 digest (asterisks denote CYP2C19 tryptic peptides); panel D—CYP2E1 digest (asterisks denote CYP2E1 tryptic peptides).

FIG. 7 shows absolute quantitation standard curves: panel A—CYP1A2 standard curve; panel B—CYP2E1 standard curve; panel C—CYP2C19 standard curve. Each data point represents the average±S.D. of data collected in six experiments.

FIG. 8 is a representative tryptic peptide mass fingerprint MALDI TOF mass spectrum of a combined tryptic digest of CYP1A2, CYP2C19 and CYP2E1.

FIG. 9 are the results of ELISA showing interaction of major isozyme-specific unique tryptic peptides of CYP2E1 (A), CYP1A2 (B), CYP2B1 (C), CYP2B2 (D) and CYP2C19 (E) with monospecific antibodies against CYP2E1 isozyme-specific tryptic peptide separately and in the mixture—panel F. Three different dilution of Ab were used (1:2000, 1:5000 and 1:10000). Amount of synthetic peptides used in each experiment ranged from 17.4 fmol to 120 pmol.

FIG. 10 is a Dot-Blot titration of CYP2E1 tryptic peptide.

FIG. 11 shows optimization of ELISA conditions for the analysis of interaction of monospecific antibodies against CYP2E1 isozyme-specific unique tryptic peptide with the major isozyme-specific unique tryptic peptide of CYP2E1 alone (A), in mixture with major isozyme-specific unique tryptic peptides of CYP2E1, CYP1A2, CYP2B1, CYP2B2 and CYP2C19 (B), and CYP2E1 (C). Red line corresponds to optimal conditions for antibody-antigen interaction conditions.

FIG. 12 is an ELISA showing the interaction of monospecific antibodies against CYP2E1 isozyme-specific unique tryptic peptide with major CYP2E1 isozyme-specific unique tryptic peptide (blue triangles), CYP2E1 (pink circles) and mixture of five major isozyme-specific unique tryptic peptides of CYP2E1, CYP1A2, CYP2B1, CYP2B2 and CYP2C19 (red triangles).

FIG. 13 shows the specificity of binding of CYP2E1 isozyme-specific unique tryptic peptide antibodies (Ab dilution 1:5000). The lanes are: 1, CYP2E1 (1 pmol); 2, CYP2E1 (600 fmol); 3, CYP2E1 (240 fmol); 4, CYP2E1 (100 fmol); 5, CYP1A2 (2.1 pmol); 6, CYP2A6 (2.1 pmol); 7, CYP2A13 (2.1 pmol); 8, CYP2C19 (2.1 pmol); 9, mixture of CYP2B1 and CYP2B2 (2.1 pmol).

FIG. 14 shows the effect of CYP2E1 isozyme-specific unique tryptic peptide antibodies on chlozoxazone 6-hydroxylation in human liver microsomes.

FIG. 15 is a Western blot of CYP1A2 isozyme-specific unique tryptic peptide antibodies. The lanes are: 1, CYP1A2 (15 pmol); 2, CYP1A2 (8.1 pmol); 3, CYP1A2 (3.2 pmol); 4, CYP1A2 (1.2 pmol).

FIG. 16 shows the ELISA results of interaction of major isozyme-specific unique tryptic peptides of CYP2E1, CYP1A2, CYP2B1, CYP2B2 and CYP2C19 with polyclonal monospecific antibodies against the CYP1A2 isozyme-specific unique tryptic peptide separately and in the mixture with different peptides under varying experimental conditions.

FIG. 17 shows the MALDI TOF mass spectra before (A) and after (B) incubation of CYP2E1 anti-peptide antibody with a mixture of different CYP isozyme-specific unique tryptic peptides alone and spiked in BSA digest (C vs D).

FIG. 18 shows the MALDI TOF spectra obtained following elution of CYP1A2 (1071 Da) and CYP2E1 (1694 Da) isozyme-specific unique-tryptic peptides from magnetic beads with immobilized polyclonal antibodies.

FIG. 19 are the absolute quantitation standard curves: panel A—CYP2E1 standard curve; panel B—CYP1A2 standard curve. Each data point represents the average±S.D. of data collected in six experiments.

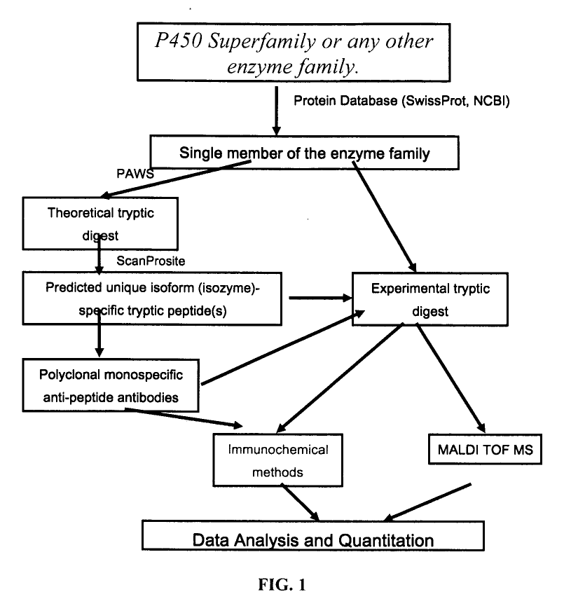

### DETAILED DESCRIPTION OF PREFERRED EMBODIMENT

The present invention involves the use of mass spectrometry and immunochemical methods to accurately measure the amounts of proteins in samples, including the situation where multiple distinct cytochrome P450 isozymes are present in the same sample. The isozymes are highly homologous and very difficult to distinguish by conventional means, yet are quite amenable to evaluation by the present invention.

From the studies illustrated herein, it was demonstrated that unique tryptic peptides derived from the isoforms/isozymes, when present in a sample, will produce MALDI-TOF MS signals that are proportional to the relative concentrations of those unique tryptic peptides. This relationship holds for the reflector mode of MALDI-TOF MS, when signals are measured by both peak intensity or peak area. Thus, MALDI-TOF MS can also be used to measure the relative amounts of closely related protein isoform s/isozymes.

The unique tryptic peptides are also useful for immunochemical detection methods of isoforms/isozymes. Antibodies raised against the unique tryptic peptides have been found to be highly specific for the isoform/isozyme of interest, and can be incorporated into various immunoassays for detection, and ultimate quantitation of the isoform/isozyme of interest. The antibodies against the unique tryptic peptides may also exhibit an inhibitory action, e.g., inhibition of the enzymatic action of the cytochrome P450 isozyme.

Definitions

The following definitions are provided for specific terms which are used in the following written description.

As used herein, the singular form “a”, “an” and “the” includes plural references unless the context clearly dictates otherwise. For example, the term “a protein” includes a plurality of proteins.

As used herein, the term “antibody” embraces a polypeptide substantially encoded by an immunoglobulin gene or immunoglobulin genes, or fragments thereof, which specifically binds and recognizes an epitope (e.g., a unique proteolytic peptide). The recognized immunoglobulin genes include the kappa and lambda light chain constant region genes, the alpha, gamma, delta, epsilon and mu heavy chain constant region genes, and the myriad immunoglobulin variable region genes. Antibodies exist as intact immunoglobulins or as a number of well characterized fragments produced by digestion with various peptidases. This includes, for example, Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. Most preferably, the antibodies of the present invention are polyclonal monospecific antibodies.

As used herein, the term “detecting” embraces the act of determining the presence, absence, or amount of a compound (e.g., the amount of the unique proteolytic peptide or unique tryptic peptide) in a sample, and can include quantifying the amount of the compound in a sample.

As used herein, an “immunoassay” embraces an assay that uses an antibody to specifically bind an antigen (e.g., the unique proteolytic peptide). The immunoassay is characterized by the use of specific binding properties of a particular antibody to isolate, target, and/or quantify the antigen. The immunoassay typically includes contacting a test sample with an antibody that specifically binds the antigen, and detecting the presence of a complex of the antibody bound to the antigen in the sample. The immunoassay procedure may be selected from a wide variety of immunoassay procedures known to the art involving recognition of antibody/antigen complexes, including enzyme immunoassays, competitive or non-competitive, and including enzyme-linked immunosorbent assays “(ELISA)”, radioimmunoassays “(RIA)” and Western blots. Such assays are well known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

As used herein, the term “distinctive proteolytic peptide” or “unique proteolytic peptide” embraces a compound comprised of subunit amino acids linked by peptide bonds generated by proteolytically cleaving a protein with a protease, which differs from any other proteolytic peptide derived from digestion of other proteins using the same protease. Preferably, the protease used to generate the unique proteolytic peptide is a serine protease, and most preferably the protease is trypsin. In such a case, the unique proteolytic peptide may be known as a “unique tryptic peptide.” Preferably, the distinctiveness or uniqueness refers to the entire genome, and most preferably to the human genome, when referenced against the SwissProt or NCBI databases. The peptide's boundaries may determined by predicting the cleavage sites of a protease. In another aspect, a protein is digested by the protease and the actual sequence of one or more peptide fragments is determined. The “unique proteolytic peptide” is preferably at least about 6 amino acids. The size of the “unique proteolytic peptide” is also optimized to maximize ionization frequency. Thus, unique proteolytic peptides longer than about 20 amino acids are not preferred. In one aspect, an optimal unique proteolytic peptide ranges from about 6 amino acids to about 20 amino acids, and preferably from about 7 amino acids to about 15 amino acids.

As used herein, the term “isoform” embraces different forms of a protein encoded by related forms or alleles of a gene located at the same or at different loci as, for example, the different forms of the cytochrome P450 family of proteins. The term also embraces a family of related proteins (i.e. multiple forms of the same protein) that differ somewhat in their amino acid sequence. They can be produced by different genes or by alternative splicing of RNA transcripts from the same gene. Thus, the term “isoform” comprises homologous sequences of amino acid residues interspersed with variable sequences. Also, the term “isoform” comprises a form of the protein which has been post translationally processed, e.g., phosphorylated (phospho-isoform). When the proteins which function as enzymes are involved, the isoform may be denominated as an “isozyme.”

As used herein, the term “monospecific” embraces antibodies that do not have any epitopes for antigens other than the unique proteolytic peptide.

As used herein, the term “sample” embraces any quantity of a substance from a living thing or formerly living thing. Such living things include, but are not limited to, humans, mice, monkeys, rats, rabbits, and other mammals. Such substances include, but are not limited to, blood, serum, urine, cells, organs, tissues, bone, bone marrow, lymph nodes, and skin.

As used herein, the term “protein” embraces any protein, including, but not limited to peptides, enzymes (e.g., P450s), hormones, receptors, antigens, antibodies, growth factors, etc., without limitation. The terms “polypeptide” and “protein” are generally used interchangeably herein to refer to a polymer of amino acid residues.

As used herein, a “protein of interest” is a protein whose presence or amount is being determined in a protein sample. The protein/polypeptide may be a known protein (i.e., previously isolated and purified) or a putative protein (i.e., predicted to exist on the basis of an open reading frame in a nucleic acid sequence).

As used herein, “a protease cleavage site” refers to an amide bond which is broken by the action of a protease.

As used herein, the term “reporter group” embraces enzymatic groups, photochemically reactive groups, chromophoric or fluorophoric groups, luminescent groups, radioactive groups, paramagnetic ions, thermochemically reactive groups, and one part of an affinity pair. Examples enzymatic groups include horseradish peroxidase, alkaline phosphatase, and beta-galactosidase. Detection agents for reporter groups generally utilize a form of the enzyme's substrate. The substrate is typically modified, or provided under a set of conditions, such that a chemiluminescent, colorimetric, or fluorescent signal is observed after the enzyme and substrate has been contacted (Vargas et al., Anal. Biochem. 209: 323, 1993). Examples photochemically reactive groups include substituted coumarins, benzofurans, indols, angelicins, psoralens, carbene and nitrene precursors, ketones, and quinones, e.g., anthraquinones (AQ), phenanthraquinones and benzoquinonones. Examples of chromophoric and fluorophoric reporters include groups having an extensive delocalized electron system, eg. cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes, etc. Examples of suitable organic or metallated organic chromophores may be found in “Topics in Applied Chemistry: Infrared absorbing dyes” Ed. M. Matsuoka, Plenum, N.Y. 1990, “Topics in Applied Chemistry: The Chemistry and Application of Dyes”, Waring et al., Plenum, N.Y., 1990, “Handbook of Fluorescent Probes and Research Chemicals” Haugland, Molecular Probes Inc, 1996, DE-A-4445065, DE-A-4326466, JP-A-3/228046, Narayanan et al. J. Org. Chem. 60: 2391-2395 (1995), Lipowska et al. Heterocyclic Comm. 1: 427-430 (1995), Fabian et al. Chem. Rev. 92: 1197 (1992), WO96/23525, Strekowska et al. J. Org. Chem. 57: 4578-4580 (1992), WO (Axis) and WO96/17628. Particular examples of chromophores and fluorophores which may be used include xylene cyanole, fluorescein, dansyl, NBD, indocyanine green, DODCI, DTDCI, DOTCI and DDTCI. Examples of fluorescent groups include umbelliferone, fluorescein, fluorescein isothiocyanate, rhodamine, dichlorotriazinylamine fluorescein, dansyl chloride, Cy-dyes, Alexa-dyes or phycoerythrin. Examples of luminescent groups include luminol, luciferase, luciferin, and aequorin. Examples of radioactive groups are ¹²⁵I, ¹³¹I, ³⁵S or ³H. Examples of the paramagnetic groups include those containing chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Examples of thermochemically reactive groups include carboxylic acids, primary amines, secondary amines, acid hydrazides, semicarbazides, thiosemicarbazides, thiols, aliphatic hydrazines, aromatic hydrazines, epoxides and maleimides. Examples one part of an affinity pair (preferably the part having the lower molecular weight, e.g., a molecular weight of up to 7,000) include one part of biotin/avidin, biotin/streptavidin, biotin/NeutrAvidin, glutathione/glutathione-S-transferase. Preferably, the reporter group comprises a biotin (a part of an affinity pair).

It will be appreciated from the foregoing that some of these reporter groups can be detected directly or indirectly. For example, fluorescent groups can be directly detected with a suitable detection device, such as a fluorescent microscope. Similarly, radioisotopes can be detected through the use of a scintillation counter or Geiger counter. Other reporter groups can be detected indirectly. These reporter groups may require the use of a suitable detection agent. The choice of a suitable detection agent generally depends on which detectable label is used. For example, if a protein such as biotin is used as the reporter group, a detection agent comprising avidin or streptavidin may generally employed (Bayer et al., Meth. Biochem. Anal. 26: 1-10, 1980).

Mass Spectrometry

One skilled in the art will recognize that measurement of the unique proteolytic peptides (e.g., the unique tryptic peptides) may be accomplished by mass spectrometry. For a general discussion of mass spectrometry and its application to biotechnology see Mass Spectrometry for Biotechnology (1996). In addition, MALDI-TOF techniques are discussed in Perryman et al., U.S. Published Patent 2004/0119010 entitled “Quantitative analysis of protein isoforms using matrix-assisted laser desorption/ionization time of flight mass spectrometry,” which is incorporated by reference.

Certain Antibody Uses

According to certain embodiments, the antibodies of the present invention are useful for detecting a particular antigen (protein of interest or unique proteolytic peptide, such as a unique tryptic peptide) in a sample. In certain embodiments, this allows the identification of cells or tissues which produce the protein. For example, in certain embodiments, antibodies against CYP2E1 unique tryptic peptides may be used to detect the presence or absence of the CYP2E1 enzyme in a sample. Similarly, antibodies against CYP1A2 unique tryptic peptides may be used to detect the presence or absence of the CYP1A2 enzyme in a sample.

In certain embodiments, a method for detecting the presence or absence of CYP2E1 enzyme in a sample comprises (a) combining an antibody against a CYP2E1 unique tryptic peptide and the sample; (b) separating antibodies bound to an antigen from unbound antibodies; and (c) detecting the presence or absence of antibodies bound to the antigen. Similarly, in certain embodiments, a method for detecting the presence or absence of CYP1A2 enzyme in a sample comprises (a) combining an antibody against a CYP1A2 unique tryptic peptide and the sample; (b) separating antibodies bound to an antigen from unbound antibodies; and (c) detecting the presence or absence of antibodies bound to the antigen.

Assays in which an antibody may be used to detect the presence or absence of an antigen include, but are not limited to, an ELISA and a Western blot. In certain embodiments, a unique proteolytic peptide antibody (e.g., antibody against a unique tryptic peptide) may be labeled with a reporter group. In certain embodiments, a kit for detecting the presence or absence of a protein of interest (such as a cytochrome P450 isozyme) in a sample is provided. In certain embodiments, the kit comprises an polyclonal monospecific antibody against a unique proteolytic peptide for the protein of interest (such as a unique tryptic peptide for a cytochrome P450 isozyme) and reagents for detecting the antibody.

In certain embodiments, antibodies may be used to substantially isolate a protein of interest. In certain embodiments, the antibody is attached to a “substrate,” which is a supporting material used for immobilizing the antibody. Substrates include, but are not limited to, tubes, plates (i.e., multi-well plates), beads such as microbeads, filters, balls, and membranes. In certain embodiments, a substrate can be made of water-insoluble materials such as, but not limited to, polycarbonate resin, silicone resin, or nylon resin. Exemplary substrates for use in affinity chromatography include, but are not limited to, cellulose, agarose, polyacrylamide, dextran, polystyrene, polyvinyl alcohol, and porous silica. There are many commercially available chromatography substrates that include, but are not limited to, Sepharose 2b, Sepharose 4B, Sepharose 6B and other forms of Sepharose (Pharmacia); Bio-Gel (and various forms of Bio-Gel such as Biogel A, P, or CM), Cellex (and various forms of Cellex such as Cellex AE or Cellex-CM), Chromagel A, Chromagel P and Enzafix (Wako Chemical Indus.). The use of antibody affinity columns is known to a person of ordinary skill in the art. In certain embodiments, a method for isolating the protein of interest comprises (a) attaching an antibody raised against a unique tryptic peptide for the protein of interest to a substrate; (b) exposing a sample containing the protein of interest to the antibody of part (a); and (c) isolating the protein of interest. In certain embodiments, a kit for isolating a protein of interest is provided. In certain embodiments, the kit comprises an antibody raised against a unique tryptic peptide for the protein of interest, the antibody attached to a substrate and reagents for isolating protein of interest. In certain embodiments, the kit comprises polyclonal monospecific antibodies against a CYP2E1 unique tryptic peptide attached to a substrate and reagents for isolating CYP2E1 from a sample. In certain embodiments, the kit comprises polyclonal monospecific antibodies against a CYP1A2 unique tryptic peptide attached to a substrate and reagents for isolating CYP1A2 from a sample.

It will be appreciated that in the immunoassays of the present invention, after incubating the test sample with the antibody, the mixture is washed and the antibody-marker complex may be detected. The detection can be accomplished by incubating the washed mixture with a detection reagent, and observing, for example, development of a color or other indicator. The detection reagent may be, for example, a second antibody which is labeled with a detectable label. Exemplary detectable labels include magnetic beads (e.g., DYNABEADS), fluorescent dyes, radiolabels, enzymes (e.g., horseradish peroxide, alkaline phosphatase and others commonly used in enzyme immunoassay procedures), and colorimetric labels such as colloidal gold, colored glass or plastic beads. Alternatively, the marker in the sample can be detected using an indirect assay, wherein, for example, a second, labeled antibody is used to detect bound marker-specific antibody. The amount of an antibody-marker complex can be determined by comparing to a standard.

### EXAMPLE 1

### Selection of Distinctive or Unique Proteolytic Peptides from CYP2B1 and CYP2B2

In this present invention, it was shown that CYP isozyme-specific unique tryptic peptides peak height, or peak area, ratios obtained by MALDI-TOF MS could reflect protein molar ratios in the digested samples. The first step in this process involves the selection of the unique proteolytic peptides for ultimate quantification. Two very closely related cytochrome P450 isozymes, CYP2B1 and 2B2, were chosen for this example. CYP2B1 is the major form of P450 induced in the liver of adult rats after exposure to phenobarbital (“PB”). PB also induces CYP2B2, but it is not clear how extensively.

The isozymes CYP2B1 and CYP2B2 are highly similar (greater than 97%) differing in only 14 amino acids out of 491. Their theoretical tryptic digests differ in five pairs of peptides, and four pairs of those peptides fall within the optimal MALDI working range, 800-2500 amu, as shown in the following table.

SEQ

| | | |
|---|---|---|
| ID | CYP | MH⁺ |

| | | | | |
|---|---|---|---|---|
| NO. | isozyme | Start-End | Peptide Sequence | (calculated) |

| | | | | |
|---|---|---|---|---|
| 495 | CYP2B1 | 1-21 | MEPTILLLLALLVGFL | 2350.484 |
| | | | LLLVR | |
| 496 | CYP2B2 | 1-21 | MEPSILLLLALLVGFL | 2336.468 |
| | | | LLLVR | |
| 497 | CYP2B1 | 317-323 | YPHVAEK | 843.429 |
| 498 | CYP2B2 | 317-323 | YPHVTEK | 873.439 |
| 499 | CYP2B1 | 327-336 | EIDQVIGSHR(LPTLD | 1153.589 |
| | | | DR) | |
| 500 | CYP2B2 | 327-343 | EIDQVIGSHRPPSLDD | 1933.965 |
| | | | R | |
| 501 | CYP2B1 | 359-370 | FSDLVPIGVPHR | 1336.738 |
| 502 | CYP2B2 | 359-370 | FADLAPIGLPHR | 1306.719 |

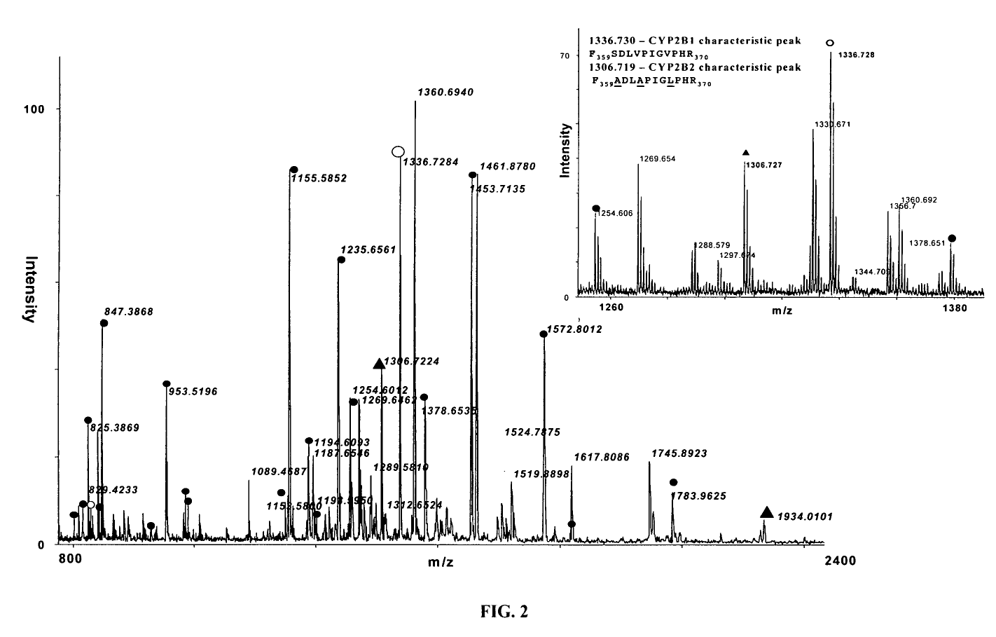

The first pair of peptides that originate from N-terminus (positions 1-21) were rarely found in experimental digests of purified CYPs or microsomal fractions (FIG. 2). One of the peptides in the second pair (317-323) has a molecular weight that differs in 1 amu from one of the self-digest fragments of trypsin (842.439 vs. 841.502) and cannot be a reliable indicator because of the overlap of resolved isotopomers. The third pair of peptides presents an interesting case. The CYP2B2 sequence contains Arg followed by Pro and as a result there is a missed cleavage in this position. In CYP2B1, Arg is followed by Leu and then by Pro, creating a more accessible cleavage site. However, in many experiments, the 1964.0 peak corresponded to a missed cleavage. Finally, a fourth pair of tryptic peptides was thus used as the isozyme-specific unique tryptic peptides and was selected for further experiments. Further, since the selected peptides originate from the same part of the molecule, position 359-370 (FIG. 2, inset) there should not be any doubt in “equal accessibility” to tryptic digest. The mass peaks of the chosen peptides were among the strongest peaks in over hundred of rat liver microsomal digests performed to date and their identity was confirmed by MS/MS (data not shown).

### EXAMPLE 2

### Quantitative Analysis by Correlation Mass Peak Area to Molar Content

In this example, it was shown that CYP isozyme-specific unique tryptic peptide's peak height, or peak area, ratios obtained by MALDI-TOF MS could reflect protein molar ratios in the digested samples.

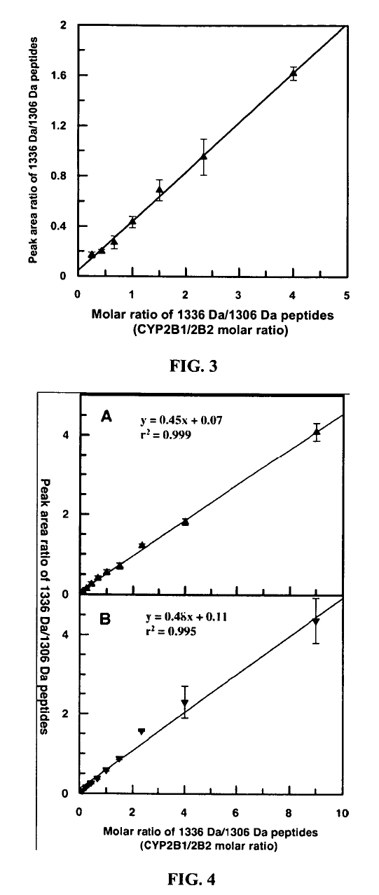

The selected tryptic peptides for CYP2B1 and CYP2B2 (SEQ. ID NO. 501 and 502) were synthesized, mixed in different ratios and analyzed by MALDI-TOF MS. FIG. 3 shows that the molar ratio of isozyme-specific unique tryptic peptides is linearly proportional to the mass peak area ratio of corresponding peptides (trend line R²=0.993).

Several factors related to sample preparation and some instrument-related parameters are known to contribute to difficulties associated with quantitative MALDI TOF MS applications. Most significant factors are heterogeneity of analyte crystallization (Cohen and Chait 1996; Figueroa, Torres et al. 1998; Garden and Sweedler 2000), and control of ion suppression effects (Kratzer, Eckerskorn et al. 1998; Knochenmuss, Dubois et al. 1999). In this example, the data acquisition conditions were optimized to control reproducibility. First, a 400 well target plate with Teflon coating was used. These plates have well areas smaller and better defined than in other types of targets. As a result, a more concentrated distribution of crystals was achieved and laser-firing patterns needed to cover less area. Furthermore to compensate for the heterogeneity of the analyte crystallization and to cover as much target area as possible, a spiral-firing pattern was used when laser beam was moved from crystal area to crystal area with 4-5 laser shots at each firing position (total 100 shots/spectrum). Next, the matrix-to-analyte ratio (v/v) was kept constant for all points in the same experimental series. Then, the high voltage was turned on at least 40 minutes before start of a data acquisition to stabilize laser power and all samples were analyzed at the same laser power adjusted so that it would not produce saturated signals of analytes while producing analyte peaks with signal-to-noise ratio greater than 5. Finally, the effect of laser shots per spectrum on linearity of analytes signal area ratios was analyzed, and it was found that 100 shots/spectrum provided better correlation coefficients, although all correlation coefficients obtained were in a good range (from 0.97 for 500 shots to 0.99 for 100 shots).

### EXAMPLE 3

### Ion Suppression Effect

The evaluation of the ion suppression effect was performed by spiking digests of bovine serum albumin (BSA) and beta-lactoglobulin A (β-LGA) with synthesized CYP2B1 and CYP2B2 isozyme-specific unique tryptic peptides (SEQ. ID NO. 501 and 502) in various ratios. In both cases a linear response between the molar ratio and the corresponding mass peak areas was observed. FIG. 4 illustrates such dependence for digests of BSA (panel A) and β-LGA (panel B) spiked with synthesized CYP2B1 and CYP2B2 isozyme-specific unique tryptic peptides.

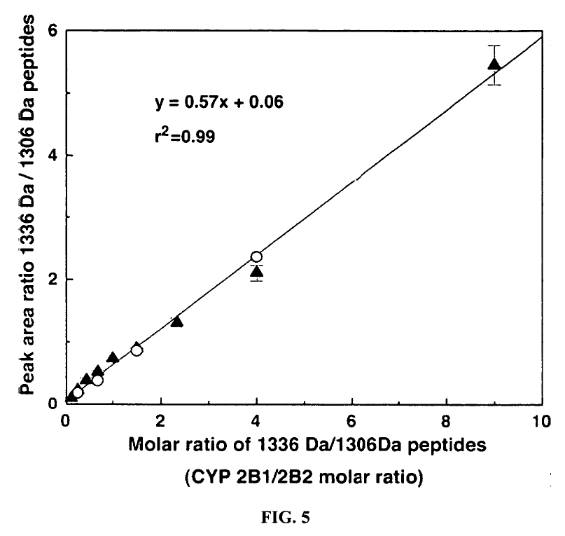

Next, the developed method was applied to the microsomal sample separated on SDS-PAGE gel. Rat liver microsomes were obtained from untreated male rats. Previously it was shown that such microsomes do not contain CYP2B1 and CYP2B2 (Galeva and Altermann 2002; Galeva, Yakovlev et al. 2003; Nisar, Lane et al. 2004). Twenty μg of total microsomal protein were electrophoresed on 10% SDS PAGE. Several bands with an apparent molecular mass of 50-60 KDa were excised and subjected to tryptic digest. The band containing CYP2D2 (sequence identity to CYP2B1 and CYP2B2 41%) was chosen for further experiments. To determine the relative amounts of CYP2B1 and CYP2B2 a calibration curve was developed using corresponding synthetic isozyme-specific unique tryptic peptides (FIG. 5). Then, the tryptic digest of CYP2D2 was spiked with these two peptides in different ratios to simulate digests of CYP2B1 and CYP2B2 and analyzed by MALDI TOF. As is seen from FIG. 5, there was a very good correlation between the experimental points (circles) and the calibration curve.

### EXAMPLE 4

### Selection of Other Unique Proteolytic Peptides from CYP Isozymes

Based on the results obtained from experiments with CYP2B1 and CYP2B2, the PMF MALDI TOF-based quantitative approach was applied to other CYP isozymes and particularly to human CYPs. The human genome encodes 57 cytochrome P450 genes. Thirty-five of these genes encode P450s belonging to families 1 to 4 (Danielson 2002). CYPs associated with families 1 to 3 are the key enzymes of Phase I in human drug metabolism, while members of CYP4 family are mainly involved in fatty acid and arachidonic acid metabolism. The remaining 14 CYP families for the most part are implicated in steroid metabolism. First of all, considering large number of human CYPs and high degree of homology between members of CYP subfamilies, a search was performed to determine if all of human CYPs possess unique isozyme-specific unique tryptic peptides. To this end, a database search was performed for unique isozyme-specific unique tryptic peptides of human P450s.

The following set of requirements was considered in this search. First, suitable unique tryptic peptide candidates do not have any similar counterparts (homologues) preferably in any organism, or, at least, in humans. The peptides preferably have a mass between 900 and 1900 Da to achieve best possible accuracy and resolution in MALDI TOF spectrum. In addition, the peptides preferably have an Arg at the C-terminus since Arg-ending peptides produce much stronger MS signals in MALDI TOF MS than Lys-ending peptides. Finally, the peptides preferably do not contain any missed cleavages. A list of isozyme-specific unique tryptic peptides was developed using PAWS software (Genomic Solutions) to generate simulated tryptic digests and ScanProsite search engine (http://au.expasy.org/tools/scanprosite) to scan protein sequences from Swiss-Prot, TrEMBL and PDB with a user-entered pattern (in our case candidate tryptic peptides).

Based on these parameters, it was determined that all human CYPs have from 2 to 14 isozyme-specific unique tryptic peptides, and the complete list encompasses hundreds of peptides. The following table shows predicted unique isozyme-specific unique tryptic peptides, including three human CYPs (CYP1A2, CYP2E1, and CYP2C19) that were used in further experiments. The asterisk designates that the unique tryptic peptide exhibits a dominant peak in MALDI-TOF.

SEQ.
ID

| | | | | | |
|---|---|---|---|---|---|
| NO. | CYP | Start | Finish | Sequence | Mass |

| | | | | | |
|---|---|---|---|---|---|
| 1 | 1A1 | 66 | 77 | MSQQYGDVLQIR | 1436.708 |
| 2 | 1A1 | 242 | 252 | YLPNPSLNAFK | 1262.666 |
| 3 | 1A1 | 293 | 306 | QLDENANVQLSDEK | 1601.753 |
| 4 | 1A1 | 343 | 353 | IQEELDTVIGR | 1271.762 |
| 5 | 1A1 | 363 | 377 | SHLPYMEAFILETFR | 1852.918 |
| 6 | 1A1 | 378 | 392 | HSSFVPFTIPHSTTR | 1712.863 |
| 7 | 1A1 | 420 | 431 | LWVNPSEFLPER | 1485.762 |
| 8 | 1A1 | 432 | 441 | FLTPDGAIDK | 1075.555 |
| 9 | 1A1 | 465 | 477 | WEVFLFLAILLQR | 1646.955 |
| 10 | 1A1 | 478 | 487 | VEFSVPLGVK | 1073.612 |
| 11 | 1A1 | 488 | 499 | VDMTPIYGLTMK | 1367.683 |
| 12 | 1A2 | 80 | 90 | IGSTPVLVLSR | 1140.687 |
| 13 | 1A2 | 244 | 252 | YLPNPALQR | 1070.587* |
| 14 | 1A2 | 267 | 277 | TVQEHYQDFDK | 1408.626 |
| 15 | 1A2 | 297 | 306 | ASGNLIPQEK | 1055.561 |
| 16 | 1A2 | 378 | 392 | HSSFLPFTIPHSTTR | 1726.879 |
| 17 | 1A2 | 393 | 403 | DTTLNGFYIPK | 1267.645 |
| 18 | 1A2 | 432 | 447 | FLTADGTAINKPLSEK | 1703.909 |
| 19 | 1A2 | 489 | 500 | VDLTPIYGLTMK | 1349.726 |
| 20 | 1B1 | 131 | 142 | SMAFGHYSEHWK | 1478.640 |
| 21 | 1B1 | 164 | 175 | QVLEGHVLSEAR | 1336.710 |
| 22 | 1B1 | 214 | 222 | YSHDDPEFR | 1164.484 |
| 23 | 1B1 | 223 | 233 | ELLSHNEEFGR | 1329.631 |
| 24 | 1B1 | 267 | 275 | NFSNFILDK | 1096.555 |
| 25 | 1B1 | 291 | 302 | DMMDAFILSAEK | 1369.626 |
| 26 | 1B1 | 356 | 366 | VQAELDQVVGR | 1212.646 |
| 27 | 1B1 | 434 | 444 | WPNPENFDPAR | 1341.610 |
| 28 | 1B1 | 503 | 514 | MNFSYGLTIKPK | 1397.738 |
| 29 | 1B1 | 524 | 539 | ESMELLDSAVQNLQAK | 1774.877 |
| 30 | 2A6 | 149 | 161 | IQEEAGFLIDAHR | 1497.757 |
| 31 | 2A6 | 162 | 176 | GTGGANIDPTFFLSR | 1551.768 |
| 32 | 2A6 | 388 | 400 | GTEVYPMLGSVLR | 1420.738* |
| 33 | 2A7 | 149 | 161 | IQEESGFLIEAIR | 1503.793 |
| 34 | 2A7 | 162 | 176 | SSHGANIDPTFFLSR | 1647.801 |
| 35 | 2A7 | 388 | 400 | GTEVFPMLGSVLR | 1404.743* |
| 36 | 2A13 | 102 | 112 | GEQATFDWLFK | 1340.640 |
| 37 | 2A13 | 149 | 161 | IQEEAGFLIDALR | 1473.783 |
| 38 | 2A13 | 162 | 176 | GTHGANIDPTFFLSR | 1631.806 |
| 39 | 2A13 | 240 | 250 | ELQGLEDFIAK | 1261.655 |
| 40 | 2A13 | 349 | 361 | MPYTEAVIHEIQR | 1585.792 |
| 41 | 2A13 | 362 | 373 | FGDMLPMGLAHR | 1343.648 |
| 42 | 2A13 | 388 | 400 | GTEVFPMLGSELR | 1434.718* |
| 43 | 2B6 | 101 | 109 | IAMVDPFFR | 1094.558 |
| 44 | 2B6 | 110 | 120 | GYGVIFANGNR | 1166.583 |
| 45 | 2B6 | 188 | 197 | FHYQDQEFLK | 1353.635 |
| 46 | 2B6 | 263 | 274 | DLIDTYLLHMEK | 1489.749 |
| 47 | 2B6 | 346 | 358 | MPYTEAVIYEIQR | 1611.797* |
| 48 | 2B6 | 423 | 433 | TEAFIPFSLGK | 1208.644 |
| 49 | 2C8 | 85 | 97 | EALIDNGEEFSGR | 1435.658 |
| 50 | 2C8 | 191 | 199 | DQNFLTLMK | 1108.559 |
| 51 | 2C8 | 250 | 261 | EHQASLDVNNPR | 1378.659 |
| 52 | 2C8 | 323 | 333 | VQEEIDHVIGR | 1293.668 |
| 53 | 2C8 | 384 | 399 | GTTIMALLTSVLHDDK | 1713.897* |
| 54 | 2C9 | 98 | 105 | GIFPLAER | 901.502 |
| 55 | 2C9 | 384 | 399 | GTTILISLTSVLHDNK | 1710.952* |
| 56 | 2C10 | 98 | 105 | GIFPLAER | 901.502 |
| 57 | 2C10 | 384 | 399 | GTTILISLTSVLHDNK | 1710.952* |
| 58 | 2C18 | 85 | 97 | EALIDHGEEFSGR | 1458.674 |
| 59 | 2C18 | 109 | 118 | GLGILFSNGK | 1004.565 |
| 60 | 2C18 | 233 | 241 | IAENFAYIK | 1067.565 |
| 61 | 2C18 | 250 | 261 | EHQESLDMNSAR | 1415.610 |
| 62 | 2C18 | 384 | 399 | GMTIITSLTSVLHNDK | 1728.908* |
| 63 | 2C18 | 466 | 478 | DIDITPIANAFGR | 1401.725 |
| 64 | 2C19 | 60 | 73 | IYGPVFTLYFGLER | 1673.882 |
| 65 | 2C19 | 74 | 84 | MVVLHGYEVVK | 1272.690 |
| 66 | 2C19 | 98 | 105 | GHFPLAER | 925.477 |
| 67 | 2C19 | 236 | 247 | NLAFMESDILEK | 1408.691 |
| 68 | 2C19 | 250 | 261 | EHQESMDINNPR | 1468.636 |
| 69 | 2C19 | 343 | 357 | GHMPYTDAVVHEVQR | 1737.826* |
| 70 | 2C19 | 384 | 399 | GTTILTSLTSVLHDNK | 1698.915 |
| 71 | 2C19 | 400 | 410 | EFPNPEMFDPR | 1377.602 |
| 72 | 2C19 | 422 | 432 | SNYFMPFSAGK | 1247.564 |
| 73 | 2D6 | 205 | 214 | LLDLAQEGLK | 1098.628 |
| 74 | 2D6 | 246 | 259 | AFLTQLDELLTEHR | 1684.878 |
| 75 | 2D6 | 260 | 269 | MTWDPAQPPR | 1197.560 |
| 76 | 2D6 | 270 | 281 | DLTEAFLAEMEK | 1395.659 |
| 77 | 2D6 | 284 | 296 | GNPESSFNDENLR | 1477.643 |
| 78 | 2D6 | 331 | 343 | VQQEIDDVIGQVR | 1497.779 |
| 79 | 2D6 | 366 | 380 | FGDIVPLGMTHMTSR | 1660.806 |
| 80 | 2D6 | 392 | 404 | GTTLITNLSSVLK | 1345.782 |
| 81 | 2D6 | 405 | 414 | DEAVWEKPFR | 1275.625 |
| 82 | 2E1 | 64 | 76 | FGPVFTLYVGSQR | 1469.767 |
| 83 | 2E1 | 101 | 110 | GDLPAFHAHR | 1119.557 |
| 84 | 2E1 | 113 | 123 | GIIFNNGPTWK | 1245.651 |
| 85 | 2E1 | 150 | 159 | EAHFLLEALR | 1197.651 |
| 86 | 2E1 | 188 | 195 | HFDYNDEK | 1066.436 |
| 87 | 2E1 | 345 | 359 | QEMPYMDAVVHEIQR | 1844.855 |
| 88 | 2E1 | 360 | 374 | FITLVPSNLPHEATR | 1693.915* |
| 89 | 2E1 | 409 | 420 | FKPEHFLNENGK | 1458.725 |
| 90 | 2E1 | 423 | 434 | YSDYFKPFSTGK | 1438.677 |
| 91 | 2F1 | 61 | 73 | EYGSMYTVHLGPR | 1508.708 |
| 92 | 2F1 | 75 | 85 | VVVLSGYQAVK | 1161.676 |
| 93 | 2F1 | 86 | 98 | EALVDQGEEFSGR | 1435.658 |
| 94 | 2F1 | 110 | 120 | GNGIAFSSGDR | 1079.499 |
| 95 | 2F1 | 126 | 133 | QFSIQILR | 1003.581 |
| 96 | 2F1 | 146 | 158 | ILEEGSFLLADVR | 1460.787 |
| 97 | 2F1 | 160 | 173 | TEGEPFDPTFVLSR | 1593.767 |
| 98 | 2F1 | 224 | 237 | FPSLLDWVPGPHQR | 1647.852 |
| 99 | 2F1 | 247 | 262 | DLIAHSVHDHQASSPR | 1768.860 |
| 100 | 2F1 | 302 | 316 | TVSTTLHHAFLALMK | 1668.902 |
| 101 | 2F1 | 324 | 334 | TVSTTLHHAFLALMK | 1255.677 |
| 102 | 2F1 | 344 | 358 | AAMPYTDAVIHEVQR | 1699.835 |
| 103 | 2F1 | 359 | 370 | FADIIPMNLPHR | 1422.744 |
| 104 | 2F1 | 423 | 433 | SPAFMPFSAGR | 1166.554 |
| 105 | 2J2 | 20 | 36 | TLLLGTVAFLLAADFLK | 1805.070 |
| 106 | 2J2 | 124 | 135 | NGLIMSSGQAWK | 1290.639 |
| 107 | 2J2 | 159 | 171 | IQEEAQHLTEAIK | 1508.783 |
| 108 | 2J2 | 172 | 183 | EENGQPFDPHFK | 1443.642 |
| 109 | 2J2 | 201 | 213 | FEYQDSWFQQLLK | 1730.830 |
| 110 | 2J2 | 214 | 225 | LLDEVTYLEASK | 1379.718 |
| 111 | 2J2 | 239 | 252 | FLPGPHQTLFSNWK | 1670.857 |
| 112 | 2J2 | 256 | 264 | LFVSHMIDK | 1088.569 |
| 113 | 2J2 | 344 | 356 | VIGQGQQPSTAAR | 1311.689 |
| 114 | 2J2 | 357 | 371 | ESMPYTNAVIHEVQR | 1772.851 |
| 115 | 2J2 | 372 | 382 | MGNIIPLNVPR | 1222.686 |
| 116 | 2J2 | 383 | 397 | EVTVDTTLAGYHLPK | 1642.857 |
| 117 | 2J2 | 398 | 410 | GTMILTNLTALHR | 1439.792 |
| 118 | 2J2 | 436 | 445 | EAFMPFSIGK | 1125.553 |
| 119 | 2J2 | 456 | 468 | TELFIFFTSLMQK | 1603.832 |
| 120 | 2J2 | 469 | 478 | FTFRPPNNEK | 1248.625 |
| 121 | 2J2 | 485 | 495 | MGITISPVSHR | 1196.633 |
| 122 | 2R1 | 7 | 28 | AEEGAAALGGALFLLLF | 2158.215 |
| | | | | ALGVR | |
| 123 | 2R1 | 164 | 173 | FFNDAIETYK | 1246.587 |
| 124 | 2R1 | 181 | 199 | QLITNAVSNITNLIIFGE | 2115.169 |
| | | | | R | |
| 125 | 2R1 | 249 | 259 | NAAVVYDFLSR | 1253.640 |
| 126 | 2R1 | 289 | 297 | NDPSSTFSK | 981.440 |
| 127 | 2R1 | 358 | 370 | MPYTEAVLHEVLR | 1556.802 |
| 128 | 2R1 | 397 | 412 | GTTVITNLYSVHFDEK | 1822.910 |
| 129 | 2R1 | 416 | 424 | DPEVFHPER | 1124.525 |
| 130 | 2R1 | 425 | 434 | FLDSSGYFAK | 1133.539 |
| 131 | 2R1 | 436 | 445 | EALVPFSLGR | 1087.603 |
| 132 | 2R1 | 447 | 455 | HCLGEHLAR | 1034.508 |
| 133 | 2R1 | 456 | 468 | MEMFLFFTALLQR | 1645.836 |
| 134 | 2R1 | 469 | 484 | FHLHFPHELVPDLKPR | 1981.069 |
| 135 | 2R1 | 485 | 500 | LGMTLQPQPYLICAER | 1831.932 |
| 136 | 2S1 | 89 | 101 | EALGGQAEEFSGR | 1349.621 |
| 137 | 2S1 | 144 | 154 | EGEELIQAEAR | 1243.604 |
| 138 | 2S1 | 236 | 250 | QLLHHVSTLAAFTVR | 1691.947 |
| 139 | 2S1 | 251 | 266 | QVQQHQGNLDASGPAR | 1704.829 |
| 140 | 2S1 | 267 | 275 | DLVDAFLLK | 1032.585 |
| 141 | 2S1 | 276 | 290 | MAQEEQNPGTEFTNK | 1722.572 |
| 142 | 2S1 | 335 | 347 | ELGAGQAPSLGDR | 1269.631 |
| 143 | 2S1 | 350 | 362 | LPYTDAVLHEAQR | 1511.773 |
| 144 | 2S1 | 363 | 373 | LLALVPMGIPR | 1178.721 |
| 145 | 2S1 | 408 | 416 | HPEEFNPDR | 1139.500 |
| 146 | 2S1 | 427 | 437 | HEAFLPFSLGK | 1244.655 |
| 147 | 2U1 | 1 | 19 | MSSPGPSQPPAEDPPWPA | 2002.921 |
| | | | | R | |
| 148 | 2U1 | 93 | 112 | AAGIDPSVIGPQVLLAHL | 1997.142 |
| | | | | AR | |
| 149 | 2U1 | 164 | 175 | GVVFAHYGPVWR | 1386.720 |
| 150 | 2U1 | 241 | 249 | FDYTNSEFK | 1149.498 |
| 151 | 2U1 | 304 | 311 | DHQESLDR | 998.442 |
| 152 | 2U1 | 398 | 412 | AQMPYTEATIMEVQR | 1766.833 |
| 153 | 2U1 | 439 | 451 | GTLILPNLWSVHR | 1504.851 |
| 154 | 2U1 | 452 | 466 | DPAIWEKPEDFYPNR | 1875.879 |
| 155 | 2U1 | 467 | 476 | FLDDQGQLIK | 1175.619 |
| 156 | 2U1 | 478 | 487 | ETFIPFGIGK | 1107.596 |
| 157 | 2U1 | 528 | 543 | FGLTLAPHPFNITISR | 1782.978 |
| 158 | 2W1 | 7 | 17 | FGLTLAPHPFNITISR | 1244.667 |
| 159 | 2W1 | 20 | 30 | TVVLTGFEAVK | 1162.660 |
| 160 | 2W1 | 55 | 65 | GGGIFFSSGAR | 1054.520 |
| 161 | 2W1 | 97 | 106 | CLSGQLDGYR | 1110.513 |
| 162 | 2W1 | 282 | 300 | LEDQQALPYTSAVLHEVQ | 2196.117 |
| | | | | R | |
| 163 | 2W1 | 301 | 310 | FITLLPHVPR | 1191.713 |
| 164 | 2W1 | 311 | 325 | CTAADTQLGGFLLPK | 1533.786 |
| 165 | 2W1 | 365 | 374 | EAFLPFSAGR | 1093.556 |
| 166 | 2W1 | 400 | 417 | LLPPPGVSPASLDTTPAR | 1787.978 |
| 167 | 3A3 | 105 | 114 | EPFGPVGFMK | 1107.542 |
| 168 | 3A3 | 268 | 279 | VDFLQLMIDSHK | 1444.738 |
| 169 | 3A4 | 35 | 54 | LGIPGPTPLPFLGNILSY | 2133.199 |
| | | | | HK | |
| 170 | 3A4 | 70 | 90 | VWGFYDGQQPVLAITDPD | 2392.177 |
| | | | | MIK | |
| 171 | 3A4 | 115 | 126 | SAISIAEDEEWK | 1376.646 |
| 172 | 3A4 | 243 | 249 | EVTNFLR | 877.466 |
| 173 | 3A4 | 268 | 281 | VDFLQLMIDSQNSK | 1636.813 |
| 174 | 3A4 | 390 | 402 | GWVVMIPSYALHR | 1527.802 |
| 175 | 3A4 | 476 | 491 | LSLGGLLQPEKPVVLK | 1690.039 |
| 176 | 3A5 | 116 | 127 | SAISLAEDEEWK | 1376.646 |
| 177 | 3A5 | 144 | 158 | EMFPIIAQYGDVLVR | 1749.912 |
| 178 | 3A5 | 269 | 282 | LDFLQLMIDSQNSK | 1650.829 |
| 179 | 3A5 | 380 | 390 | DVEINGVFIPK | 1229.665 |
| 180 | 3A5 | 391 | 406 | GSMVVIPTYALHHDPK | 1763.903 |
| 181 | 3A5 | 407 | 418 | YWTEPEEFRPER | 1637.747 |
| 182 | 3A5 | 476 | 491 | LDTQGLLQPEKPIVLK | 1791.050 |
| 183 | 3A7 | 116 | 127 | NAISIAEDEEWK | 1403.567 |
| 184 | 3A7 | 213 | 224 | FNPLDPFVLSIK | 1388.770 |
| 185 | 3A7 | 334 | 342 | EIDTVLPNK | 1027.556 |
| 186 | 3A7 | 391 | 406 | GVVVMIPSYVLHHDPK | 1789.955 |
| 187 | 3A7 | 479 | 492 | FGGLLLTEKPIVLK | 1526.943 |
| 188 | 3A43 | 56 | 62 | GLWNFDR | 906.435 |
| 189 | 3A43 | 116 | 127 | SALSFAEDEEWK | 1410.630 |
| 190 | 3A43 | 131 | 141 | TLLSPAFTSVK | 1162.660 |
| 191 | 3A43 | 269 | 282 | VDFFQQMIDSQNSK | 1685.772 |
| 192 | 3A43 | 391 | 406 | GLAVMVPIYALHHDPK | 1759.944 |
| 193 | 3A43 | 432 | 440 | YIPFGAGPR | 976.513 |
| 194 | 3A43 | 477 | 492 | LDNLPILQPEKPIVLK | 1829.103 |
| 195 | 4A11 | 1 | 10 | MSVSVLSPSR | 1061.554 |
| 196 | 4A11 | 34 | 41 | AVQLYLHR | 998.566 |
| 197 | 4A11 | 97 | 106 | VQLYDPDYMK | 1270.590 |
| 198 | 4A11 | 234 | 250 | NAFHQNDTIYSLTSAGR | 1893.897 |
| 199 | 4A11 | 288 | 299 | HLDFLDILLLAK | 1409.828 |
| 200 | 4A11 | 300 | 309 | MENGSILSDK | 1092.512 |
| 201 | 4A11 | 392 | 403 | ELSTPVTFPDGR | 1317.656 |
| 202 | 4A11 | 408 | 423 | GIMVLLSIYGLHHNPK | 1790.986 |
| 203 | 4A11 | 424 | 435 | VWPNPEVFDPFR | 1501.735 |
| 204 | 4A11 | 478 | 486 | FELLPDPTR | 1086.571 |
| 205 | 4B1 | 111 | 124 | APDVYDFFLQWIGR | 1725.851 |
| 206 | 4B1 | 282 | 293 | HLDFLDILLGAR | 1381.772 |
| 207 | 4B1 | 347 | 362 | EILGDQDFFQWDDLGK | 1924.884 |
| 208 | 4B1 | 376 | 385 | LYPPVPQVYR | 1230.676 |
| 209 | 4B1 | 386 | 397 | QLSKPVTFVDGR | 1345.735 |
| 210 | 4B1 | 398 | 414 | SLPAGSLISMHIYALHR | 1864.998 |
| 211 | 4B1 | 415 | 429 | NSAVWPDPEVFDSLR | 1730.826 |
| 212 | 4B1 | 439 | 451 | HPFAFMPFSAGPR | 1460.702 |
| 213 | 4B1 | 474 | 482 | FEFSLDPSR | 1096.519 |
| 214 | 4F2 | 58 | 75 | NWFWGHQGMVNPTEEGMR | 2174.941 |
| 215 | 4F2 | 76 | 89 | VLTQLVATYPQGFK | 1563.866 |
| 216 | 4F2 | 90 | 108 | VWMGPISPLLSLCHPDI | 2146.143 |
| | | | | IR | |
| 217 | 4F2 | 109 | 120 | SVINASAAIAPK | 1140.650 |
| 218 | 4F2 | 369 | 386 | EIEWDDLAHLPFLTMCMK | 2191.015 |
| 219 | 4F2 | 401 | 412 | HVTQDIVLPDGR | 1348.710 |
| 220 | 4F2 | 467 | 479 | NCIGQTFAMAEMK | 1442.636 |
| 221 | 4F3 | 34 | 47 | ILAWTYTFYDNCCR | 1767.775 |
| 222 | 4F3 | 101 | 120 | IFHPTYIKPVLFAPAAIV | 2221.303 |
| | | | | PK | |
| 223 | 4F3 | 178 | 188 | WQLLASEGSAR | 1216.620 |
| 224 | 4F3 | 391 | 400 | LHPPVPAVSR | 1071.619 |
| 225 | 4F3 | 480 | 488 | VVLGLTLLR | 982.654 |
| 226 | 4F8 | 34 | 46 | ILAWTYAFYHNGR | 1610.799 |
| 227 | 4F8 | 57 | 75 | QNWFLGHLGLVTPTEEGL | 2166.122 |
| | | | | R | |
| 228 | 4F8 | 76 | 90 | VLTQLVATYPQGFVR | 1690.941 |
| 229 | 4F8 | 91 | 108 | WLGPITPIINLCHPDIVR | 2056.129 |
| 230 | 4F8 | 109 | 120 | SVINTSDAITDK | 1262.635 |
| 231 | 4F8 | 127 | 143 | TLKPWLGDGLLLSVGDK | 1811.019 |
| 232 | 4F8 | 150 | 165 | LLTPAFHFNILKPYIK | 1914.113 |
| 233 | 4F8 | 244 | 254 | DFLYFLTPCGR | 1330.638 |
| 234 | 4F8 | 277 | 290 | TLTSQGVDDFLQAK | 1521.767 |
| 235 | 4F8 | 295 | 308 | TLDFIDVLLLSEDK | 1619.866 |
| 236 | 4F8 | 369 | 386 | EIEWDDLAQLPFLTMCLK | 2164.058 |
| 237 | 4F8 | 391 | 400 | LHPPIPTFAR | 1147.650 |
| 238 | 4F8 | 401 | 412 | GCTQDVVLPDSR | 1288.608 |
| 239 | 4F8 | 454 | 466 | SPMAFIPFSAGPR | 1376.691 |
| 240 | 4F8 | 508 | 515 | AEDGLWLR | 958.487 |
| 241 | 4F11 | 34 | 46 | VLAWTYTFYDNCR | 1650.750 |
| 242 | 4F11 | 76 | 89 | TLTQLVTTYPQGFK | 1595.856 |
| 243 | 4F11 | 170 | 177 | SVNIMHDK | 942.459 |
| 244 | 4F11 | 259 | 274 | ACHLVHDFTDAVIQER | 1852.889 |
| 245 | 4F11 | 277 | 288 | TLPTQGIDDFLK | 1346.708 |
| 246 | 4F11 | 347 | 355 | HPEYQEQCR | 1188.498 |
| 247 | 4F11 | 401 | 412 | CCTQDFVLPDGR | 1352.585 |
| 248 | 4F11 | 480 | 490 | VVLALTLLHFR | 1280.797 |
| 249 | 4F11 | 491 | 499 | ILPTHTEPR | 1062.582 |
| 250 | 4F12 | 34 | 46 | ILAWTYAFYNNCR | 1633.771 |
| 251 | 4F12 | 58 | 75 | NWFWGHLGLITPTEEGLK | 2097.068 |
| 252 | 4F12 | 109 | 120 | SITNASAAIAPK | 1142.629 |
| 253 | 4F12 | 127 | 143 | FLKPWLGEGILLSGGDK | 1829.009 |
| 254 | 4F12 | 162 | 169 | SYITIFNK | 984.528 |
| 255 | 4F12 | 178 | 188 | WQHLASEGSSR | 1256.590 |
| 256 | 4F12 | 262 | 272 | LVHDFTDAVIR | 1284.683 |
| 257 | 4F12 | 277 | 288 | TLPTQGIDDFFK | 1380.692 |
| 258 | 4F12 | 391 | 400 | LHPPAPFISR | 1133.634 |
| 259 | 4F12 | 480 | 490 | VVLALMLLHFR | 1310.790 |
| 260 | 4F12 | 491 | 499 | FLPDHTEPR | 1110.546 |
| 261 | 4F12 | 516 | 524 | VEPLNVGLQ | 967.534 |
| 262 | 4V2 | 1 | 12 | MAGLWLGLVWQK | 1400.764 |
| 263 | 4V2 | 56 | 71 | AYPLVGHALLMKPDGR | 1736.939 |
| 264 | 4V2 | 72 | 85 | EFFQQIIEYTEEYR | 1893.878 |
| 265 | 4V2 | 126 | 142 | FLEPWLGLGLLTSTGNK | 1845.004 |
| 266 | 4V2 | 208 | 221 | NIGAQSNDDSEYVR | 1566.691 |
| 267 | 4V2 | 236 | 249 | MPWLWLDLWYLMFK | 1940.972 |
| 268 | 4V2 | 260 | 272 | ILHTFTNSVIAER | 1499.810 |
| 269 | 4V2 | 273 | 283 | ANEMNANEDCR | 1265.476 |
| 270 | 4V2 | 298 | 313 | AFLDLLLSVTDDEGNR | 1776.889 |
| 271 | 4V2 | 355 | 365 | VDHELDDVFGK | 1272.599 |
| 272 | 4V2 | 366 | 376 | SDRPATVEDLK | 1229.625 |
| 273 | 4V2 | 391 | 400 | LFPSVPLFAR | 1145.660 |
| 274 | 4V2 | 401 | 412 | SVSEDCEVAGYR | 1313.556 |
| 275 | 4V2 | 416 | 428 | GTEAVIIPYALHR | 1438.793 |
| 276 | 4V2 | 432 | 443 | YFPNPEEFQPER | 1551.699 |
| 277 | 4V2 | 444 | 452 | FFPENAQGR | 1064.504 |
| 278 | 4V2 | 453 | 465 | HPYAYVPFSAGPR | 1460.720 |
| 279 | 4V2 | 487 | 495 | HFWIESNQK | 1187.572 |
| 280 | 4X1 | 1 | 9 | MEFSWLETR | 1197.549 |
| 281 | 4X1 | 60 | 68 | FIQDDNMEK | 1138.496 |
| 282 | 4X1 | 139 | 150 | LLTPGFHFNILK | 1398.802 |
| 283 | 4X1 | 151 | 161 | AYIEVMAHSVK | 1246.638 |
| 284 | 4X1 | 200 | 214 | ETNCQTNSTHDPYAK | 1707.716 |
| 285 | 4X1 | 227 | 239 | LYSLLYHSDIIFK | 1610.871 |
| 286 | 4X1 | 253 | 265 | VLNQYTDTIIQER | 1591.821 |
| 287 | 4X1 | 283 | 294 | YQDFLDIVLSAK | 1410.739 |
| 288 | 4X1 | 377 | 386 | LIPAVPSISR | 1051.639 |
| 289 | 4X1 | 431 | 439 | FSQENSDQR | 1109.474 |
| 290 | 4X1 | 440 | 452 | HPYAYLPFSAGSR | 1464.715 |
| 291 | 4X1 | 453 | 465 | NCIGQEFAMIELK | 1494.721 |
| 292 | 4X1 | 466 | 476 | VTIALILLHFR | 1294.812 |
| 293 | 4Z1 | 42 | 58 | ALHLFPAPPAHWFYGHK | 1988.021 |
| 294 | 4Z1 | 116 | 123 | ILESWVGR | 958.524 |
| 295 | 4Z1 | 138 | 150 | QIVKPGFNISILK | 1455.881 |
| 296 | 4Z1 | 151 | 161 | QIVKPGFNISILK | 1312.652 |
| 297 | 4Z1 | 167 | 176 | WEEHIAQNSR | 1268.590 |
| 298 | 4Z1 | 177 | 193 | LELFQHVSLMTLDSIMK | 2004.042 |
| 299 | 4Z1 | 226 | 238 | MNNFLHHNDLVFK | 1627.793 |
| 300 | 4Z1 | 239 | 248 | FSSQGQIFSK | 1127.561 |
| 301 | 4Z1 | 249 | 259 | FNQELHQFTEK | 1419.678 |
| 302 | 4Z1 | 282 | 292 | WDFLDILLSAK | 1319.712 |
| 303 | 4Z1 | 298 | 309 | DFSEADLQAEVK | 1350.630 |
| 304 | 4Z1 | 375 | 384 | LYAPVVNISR | 1130.645 |
| 305 | 4Z1 | 385 | 396 | LLDKPITFPDGR | 1370.756 |
| 306 | 4Z1 | 437 | 450 | IHPYAFIPFSAGLR | 1587.856 |
| 307 | 4Z1 | 451 | 463 | NCIGQHFAIIECK | 1474.706 |
| 308 | 4Z1 | 464 | 472 | VAVALTLLR | 954.623 |
| 309 | 4Z1 | 475 | 487 | LAPDHSRPPQPVR | 1468.790 |
| 310 | 5A1 | 30 | 38 | WYSTSAFSR | 1103.504 |
| 311 | 5A1 | 46 | 60 | HPKPSPFIGNLTFFR | 1756.941 |
| 312 | 5A1 | 61 | 71 | QGFWESQMELR | 1409.640 |
| 313 | 5A1 | 73 | 84 | LYGPLCGYYLGR | 1373.680 |
| 314 | 5A1 | 86 | 97 | MFIVISEPDMIK | 1421.730 |
| 315 | 5A1 | 98 | 110 | QVLVENFSNFTNR | 1566.779 |
| 316 | 5A1 | 119 | 128 | SVADSVLFLR | 1105.613 |
| 317 | 5A1 | 137 | 147 | GALMSAFSPEK | 1136.554 |
| 318 | 5A1 | 169 | 180 | YAESGDAFDIQR | 1370.610 |
| 319 | 5A1 | 249 | 257 | DELNGFFNK | 1082.503 |
| 320 | 5A1 | 277 | 286 | DFLQMVLDAR | 1206.607 |
| 321 | 5A1 | 287 | 301 | HSASPMGVQDFDIVR | 1657.788 |
| 322 | 5A1 | 302 | 315 | DVFSSTGCKPNPSR | 1493.693 |
| 323 | 5A1 | 413 | 424 | EAAQDCEVLGQR | 1317.598 |
| 324 | 5A1 | 462 | 477 | QQHRPFTYLPFGAGPR | 1870.959 |
| 325 | 5A1 | 491 | 499 | LTLLHVLHK | 1072.676 |
| 326 | 5A1 | 502 | 517 | FQACPETQVPLQLESK | 1816.903 |
| 327 | 7A1 | 75 | 87 | YVHFITNPLSYHK | 1617.830 |
| 328 | 7A1 | 113 | 131 | SIDPMDGNTTENINDTFI | 2123.968 |
| | | | | K | |
| 329 | 7A1 | 132 | 151 | TLQGHALNSLTESMMENL | 2272.094 |
| | | | | QR | |
| 330 | 7A1 | 152 | 162 | IMRPPVSSNSK | 1214.644 |
| 331 | 7A1 | 163 | 177 | TAAWVTEGMYSFCYR | 1783.770 |
| 332 | 7A1 | 178 | 190 | VMFEAGYLTIFGR | 1502.759 |
| 333 | 7A1 | 200 | 210 | AHILNNLDNFK | 1297.678 |
| 334 | 7A1 | 215 | 229 | VFPALVAGLPIHMFR | 1666.938 |
| 335 | 7A1 | 251 | 260 | ESISELISLR | 1145.629 |
| 336 | 7A1 | 261 | 275 | MFLNDTLSTFDDLEK | 1787.829 |
| 337 | 7A1 | 356 | 364 | LSSASLNIR | 959.540 |
| 338 | 7A1 | 368 | 382 | EDFTLHLEDGSYNIR | 1807.838 |
| 339 | 7A1 | 421 | 429 | TTFYCNGLK | 1045.490 |
| 340 | 7A1 | 432 | 447 | YYYMPFGSGATICPGR | 1781.790 |
| 341 | 7A1 | 484 | 498 | AGLGILPPLNDIEFK | 1595.892 |
| 342 | 7B1 | 1 | 11 | MAGEVSAATGR | 1048.497 |
| 343 | 7B1 | 51 | 63 | GWLPYLGVVLNLR | 1498.866 |
| 344 | 7B1 | 76 | 88 | QHGDTFTVLLGGK | 1371.715 |
| 345 | 7B1 | 89 | 104 | YITFILDPFQYQLVIK | 2000.120 |
| 346 | 7B1 | 122 | 130 | AFSISQLQK | 1020.560 |
| 347 | 7B1 | 149 | 162 | SLDILLESMMQNLK | 1633.842 |
| 348 | 7B1 | 163 | 171 | QVFEPQLLK | 1100.623 |
| 349 | 7B1 | 223 | 239 | FAYLVSNIPIELLGNVK | 1889.066 |
| 350 | 7B1 | 257 | 267 | MQGWSEVFQSR | 1353.614 |
| 351 | 7B1 | 311 | 319 | HPEAMAAVR | 980.486 |
| 352 | 7B1 | 334 | 343 | GSGFPIHLTR | 1083.582 |
| 353 | 7B1 | 362 | 370 | LSSYSTTIR | 1026.535 |
| 354 | 7B1 | 371 | 388 | FVEEDLTLSSETGDYCVR | 2061.920 |
| 355 | 7B1 | 437 | 448 | CYLMPFGTGTSK | 1303.594 |
| 356 | 7B1 | 487 | 501 | LLFGIQYPDSDVLFR | 1781.935 |
| 357 | 8A1 | 61 | 72 | HGDIFTILVGGR | 1283.699 |
| 358 | 8A1 | 94 | 106 | LDFHAYAIFLMER | 1624.807 |
| 359 | 8A1 | 107 | 121 | IFDVQLPHYSPSDEK | 1773.857 |
| 360 | 8A1 | 175 | 188 | AGYLTLYGIEALPR | 1535.835 |
| 361 | 8A1 | 189 | 197 | THESQAQDR | 1070.474 |
| 362 | 8A1 | 198 | 208 | VHSADVFHTFR | 1314.647 |
| 363 | 8A1 | 302 | 310 | NPEALAAVR | 939.514 |
| 364 | 8A1 | 334 | 350 | VLDSTPVLDSVLSESLR | 1828.978 |
| 365 | 8A1 | 351 | 359 | LTAAPFITR | 988.570 |
| 366 | 8A1 | 360 | 373 | EVVVDLAMPMADGR | 1501.727 |
| 367 | 8A1 | 383 | 393 | LLLFPFLSPQR | 1329.781 |
| 368 | 8A1 | 394 | 405 | DPEIYTDPEVFK | 1451.682 |
| 369 | 8A1 | 409 | 417 | FLNPDGSEK | 1005.477 |
| 370 | 8A1 | 429 | 444 | NYNMPWGAGHNHCLGR | 1825.789 |
| 371 | 8A1 | 481 | 495 | YGFGLMQPEHDVPVR | 1743.840 |
| 372 | 8B1 | 38 | 50 | GTVPWLGHAMAFR | 1441.729 |
| 373 | 8B1 | 115 | 129 | SVQGDHEMIHSASTK | 1625.747 |
| 374 | 8B1 | 156 | 171 | GWSLDASCWHEDSLFR | 1907.826 |
| 375 | 8B1 | 193 | 207 | EQDLLQAGELFMEFR | 1824.872 |
| 376 | 8B1 | 216 | 224 | FVYSLLWPR | 1179.644 |
| 377 | 8B1 | 239 | 248 | MLSVSHSQEK | 1144.555 |
| 378 | 8B1 | 249 | 263 | EGISNWLGNMLQFLR | 1776.898 |
| 379 | 8B1 | 264 | 274 | EQGVPSAMQDK | 1188.544 |
| 380 | 8B1 | 310 | 320 | EEATQVLGEAR | 1201.594 |
| 381 | 8B1 | 331 | 349 | LGALQHTPVLDSVVEETL | 2076.121 |
| | | | | R | |
| 382 | 8B1 | 359 | 367 | LVHEDYTLK | 1116.581 |
| 383 | 8B1 | 368 | 377 | MSSGQEYLFR | 1216.555 |
| 384 | 8B1 | 426 | 443 | IHHYTMPWGSGVSICPGR | 1996.940 |
| 385 | 8B1 | 480 | 492 | WGFGTMQPSHDVR | 1516.688 |
| 386 | 11A1 | 15 | 24 | GYQTFLSAPR | 1138.577 |
| 387 | 11A1 | 32 | 43 | VPTGEGAGISTR | 1143.588 |
| 388 | 11A1 | 74 | 84 | VHLHHVQNFQK | 1385.732 |
| 389 | 11A1 | 152 | 165 | VALNQEVMAPEATK | 1499.765 |
| 390 | 11A1 | 166 | 176 | NFLPLLDAVSR | 1243.692 |
| 391 | 11A1 | 189 | 204 | AGSGNYSGDISDDLFR | 1672.733 |
| 392 | 11A1 | 205 | 218 | FAFESITNVIFGER | 1628.820 |
| 393 | 11A1 | 219 | 232 | QGMLEEVVNPEAQR | 1598.772 |
| 394 | 11A1 | 265 | 276 | DHVAAWDVIFSK | 1386.693 |
| 395 | 11A1 | 277 | 289 | ADIYTQNFYWELR | 1717.810 |
| 396 | 11A1 | 361 | 378 | HQAQGDMATMLQLVPLLK | 1993.049 |
| 397 | 11A1 | 387 | 396 | LHPISVTLQR | 1162.682 |
| 398 | 11A1 | 397 | 405 | YLVNDLVLR | 1103.634 |
| 399 | 11A1 | 413 | 424 | TLVQVAIYALGR | 1302.766 |
| 400 | 11A1 | 425 | 439 | EPTFFFDPENFDPTR | 1857.821 |
| 401 | 11A1 | 452 | 460 | NLGFGWGVR | 1004.519 |
| 402 | 11B1 | 7 | 20 | AEVCMAVPWLSLQR | 1601.806 |
| 403 | 11B1 | 100 | 110 | LQQVDSLHPHR | 1328.695 |
| 404 | 11B1 | 111 | 120 | MSLEPWVAYR | 1250.612 |
| 405 | 11B1 | 144 | 156 | LNPEVLSPNAVQR | 1435.778 |
| 406 | 11B1 | 333 | 341 | NPNVQQALR | 1038.557 |
| 407 | 11B1 | 437 | 448 | NFYHVPFGFGMR | 1470.687 |
| 408 | 11B1 | 455 | 469 | LAEAEMLLLLHHVLK | 1728.996 |
| 409 | 11B1 | 470 | 482 | HLQVETLTQEDIK | 1552.810 |
| 410 | 11B2 | 7 | 20 | AEVCVAAPWLSLQR | 1541.802 |
| 411 | 11B2 | 34 | 48 | TVLPFEAMPQHPGNR | 1692.841 |
| 412 | 11B2 | 88 | 99 | MVCVMLPEDVEK | 1391.650 |
| 413 | 11B2 | 100 | 110 | LQQVDSLHPCR | 1294.645 |
| 414 | 11B2 | 111 | 120 | MILEPWVAYR | 1276.664 |
| 415 | 11B2 | 333 | 341 | NPDVQQILR | 1081.588 |
| 416 | 11B2 | 413 | 422 | NAALFPRPER | 1169.630 |
| 417 | 11B2 | 437 | 448 | NFHHVPFGFGMR | 1444.682 |
| 418 | 11B2 | 470 | 482 | LAEAEMLLLLHHVLK | 1571.819 |
| 419 | 17A1 | 30 | 45 | SLLSLPLVGSLPFLPR | 1708.029 |
| 420 | 17A1 | 46 | 55 | HGHMHNNFFK | 1267.567 |
| 421 | 17A1 | 72 | 83 | TTVIVGHHQLAK | 1302.741 |
| 422 | 17A1 | 92 | 109 | DFSGRPQMATLDIASNNR | 1991.948 |
| 423 | 17A1 | 111 | 125 | GIAFADSGAHWQLHR | 1664.817 |
| 424 | 17A1 | 127 | 136 | LAMATFALFK | 1111.610 |
| 425 | 17A1 | 212 | 222 | DSLVDLVPWLK | 1283.712 |
| 426 | 17A1 | 256 | 270 | SDSITNMLDTLMQAK | 1666.791 |
| 427 | 17A1 | 313 | 325 | WTLAFLLHNPQVK | 1565.872 |
| 428 | 17A1 | 328 | 340 | LYEEIDQNVGFSR | 1568.747 |
| 429 | 17A1 | 350 | 358 | LLLLEATIR | 1040.659 |
| 430 | 17A1 | 375 | 388 | ANVDSSIGEFAVDK | 1450.694 |
| 431 | 17A1 | 389 | 404 | GTEVIINLWALHHNEK | 1872.985 |
| 432 | 17A1 | 405 | 416 | EWHQPDQFMPER | 1598.694 |
| 433 | 17A1 | 450 | 462 | QELFLIMAWLLQR | 1659.917 |
| 434 | 17A1 | 482 | 490 | VVFLIDSFK | 1066.606 |
| 435 | 19A1 | 64 | 78 | FLWMGIGSACNYYNR | 1793.802 |
| 436 | 19A1 | 86 | 98 | VWISGEETLIISK | 1473.808 |
| 437 | 19A1 | 99 | 107 | SSSMFHIMK | 1066.494 |
| 438 | 19A1 | 119 | 129 | LGLQCIGMHEK | 1227.610 |
| 439 | 19A1 | 130 | 141 | GIIFNNNPELWK | 1443.751 |
| 440 | 19A1 | 159 | 168 | MVTVCAESLK | 1079.535 |
| 441 | 19A1 | 193 | 204 | VMLDTSNTLFLR | 1408.738 |
| 442 | 19A1 | 205 | 215 | IPLDESAIVVK | 1182.686 |
| 443 | 19A1 | 252 | 261 | DAIEVLIAEK | 1099.612 |
| 444 | 19A1 | 271 | 286 | LEECMDFATELILAEK | 1853.879 |
| 445 | 19A1 | 324 | 333 | HPNVEEAIIK | 1148.619 |
| 446 | 19A1 | 334 | 342 | EIQTVIGER | 1043.561 |
| 447 | 19A1 | 354 | 364 | VMENFIYESMR | 1417.637 |
| 448 | 19A1 | 365 | 374 | YQPVVDLVMR | 1218.643 |
| 449 | 19A1 | 376 | 388 | ALEDDVIDGYPVK | 1432.798 |
| 450 | 19A1 | 390 | 399 | GTNIILNIGR | 1069.624 |
| 451 | 19A1 | 425 | 434 | YFQPFGFGPR | 1214.587 |
| 452 | 19A1 | 461 | 472 | TLQGQCVESIQK | 1332.671 |
| 453 | 19A1 | 473 | 484 | IHDLSLHPDETK | 1403.704 |
| 454 | 19A1 | 485 | 494 | NMLEMIFTPR | 1250.615 |
| 455 | 39A1 | 34 | 47 | GWIPWIGVGFEFGK | 1591.819 |
| 456 | 39A1 | 60 | 72 | YGPIFTVFAMGNR | 1471.728 |
| 457 | 39A1 | 73 | 87 | MTFVTEEEGINVFLK | 1755.875 |
| 458 | 39A1 | 91 | 103 | VDFELAVQNIVYR | 1564.825 |
| 459 | 39A1 | 110 | 118 | NVFLALHEK | 1069.592 |
| 460 | 39A1 | 164 | 177 | HLLYPVTVNMLFNK | 1687.912 |
| 461 | 39A1 | 298 | 309 | AIMEGISSVFGK | 1237.638 |
| 462 | 39A1 | 378 | 389 | YFPEPELFKPER | 1550.777 |
| 463 | 39A1 | 398 | 411 | HSFLDCFMAFGSGK | 1545.674 |
| 464 | 39A1 | 435 | 445 | YDCSLLDPLPK | 1262.622 |
| 465 | 39A1 | 446 | 462 | QSYLHLVGVPQPEGQCR | 1909.947 |
| 466 | 46A1 | 59 | 69 | VLQDVFLDWAK | 1332.708 |
| 467 | 46A1 | 83 | 94 | TSVIVTSPESVK | 1245.682 |
| 468 | 46A1 | 111 | 119 | ALQTVFGER | 1019.540 |
| 469 | 46A1 | 120 | 133 | LFGQGLVSECNYER | 1613.751 |
| 470 | 46A1 | 148 | 160 | SSLVSLMETFNEK | 1483.723 |
| 471 | 46A1 | 161 | 171 | AEQLVEILEAK | 1241.687 |
| 472 | 46A1 | 217 | 226 | LMLEGITASR | 1089.585 |
| 473 | 46A1 | 268 | 281 | GEEVPADILTQILK | 1524.840 |
| 474 | 46A1 | 328 | 339 | LQAEVDEVIGSK | 1286.672 |
| 475 | 46A1 | 341 | 349 | YLDFEDLGR | 1126.529 |
| 476 | 46A1 | 350 | 358 | LQYLSQVLK | 1090.639 |
| 477 | 46A1 | 363 | 372 | LYPPAWGTFR | 1206.618 |
| 478 | 46A1 | 373 | 384 | LLEEETLIDGVR | 1385.740 |
| 479 | 46A1 | 385 | 400 | VPGNTPLLFSTYVMGR | 1750.908 |
| 480 | 46A1 | 401 | 415 | MDTYFEDPLTFNPDR | 1859.804 |
| 481 | 46A1 | 425 | 435 | FTYFPFSLGHR | 1370.677 |
| 482 | 46A1 | 436 | 448 | SCIGQQFAQMEVK | 1467.685 |
| 483 | 51A1 | 46 | 59 | LAAGHLVQLPAGVK | 1372.819 |
| 484 | 51A1 | 80 | 91 | SPIEFLENAYEK | 1438.698 |
| 485 | 51A1 | 92 | 103 | YGPVFSFTMVGK | 1331.658 |
| 486 | 51A1 | 122 | 133 | NEDLNAEDVYSR | 1423.621 |
| 487 | 51A1 | 142 | 156 | GVAYDVPNPVFLEQK | 1674.862 |
| 488 | 51A1 | 181 | 192 | EYFESWGESGEK | 1446.594 |
| 489 | 51A1 | 278 | 291 | IDDILQTLLDATYK | 1620.861 |
| 490 | 51A1 | 343 | 358 | TVCGENLPPLTYDQLK | 1789.892 |
| 491 | 51A1 | 373 | 382 | LRPPIMIMMR | 1256.692 |
| 492 | 51A1 | 426 | 436 | YLQDNPASGEK | 1220.567 |
| 493 | 51A1 | 437 | 446 | FAYVPFGAGR | 1083.550 |
| 494 | 51A1 | 449 | 460 | CIGENFAYVQIK | 1383.686 |

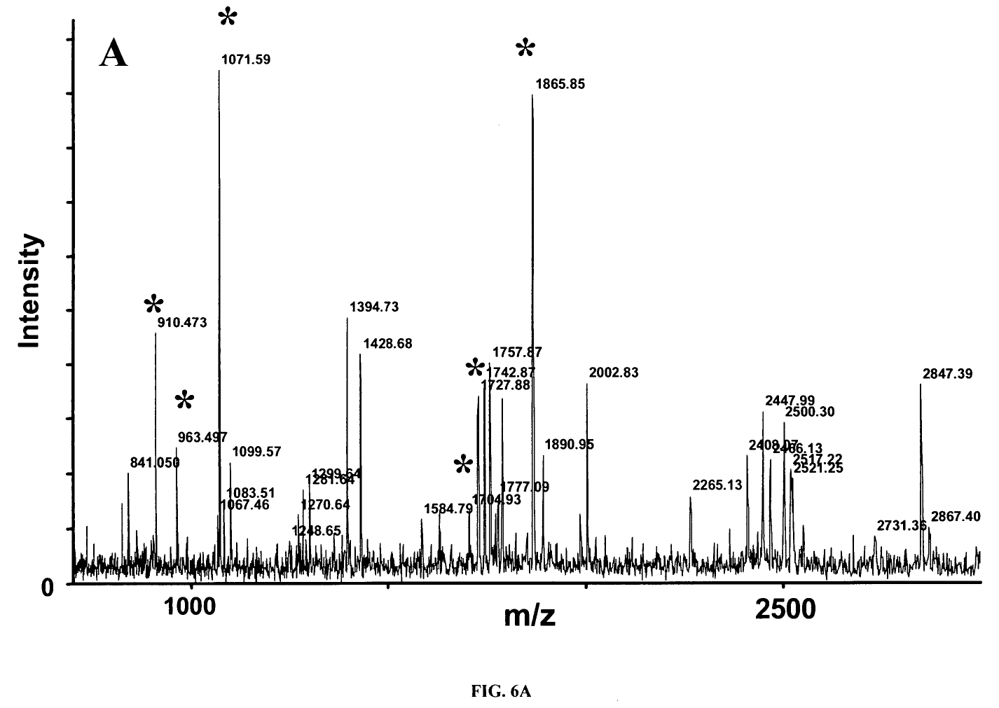

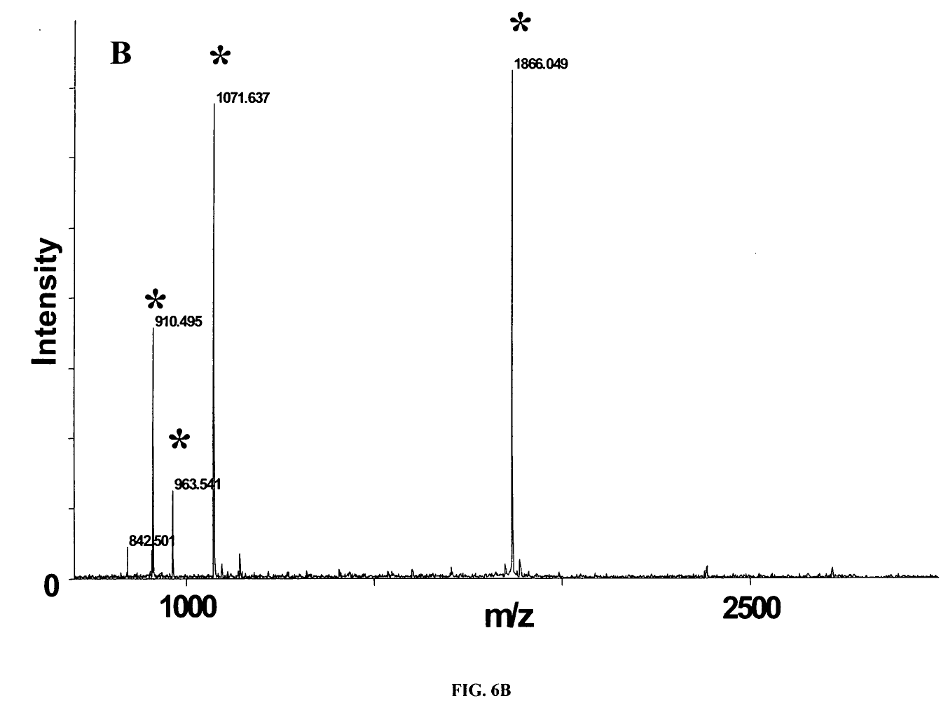

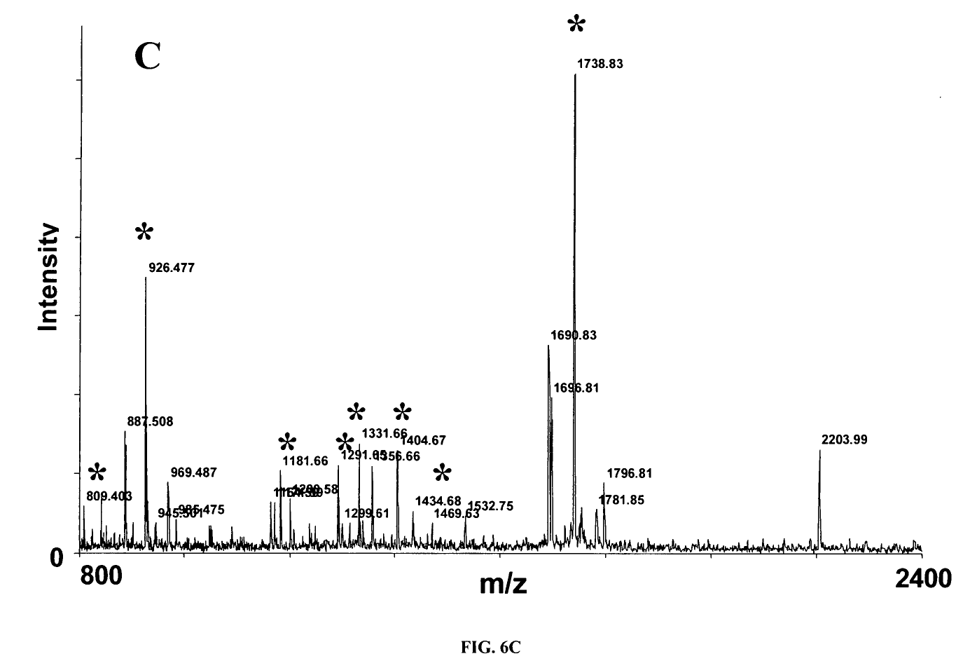

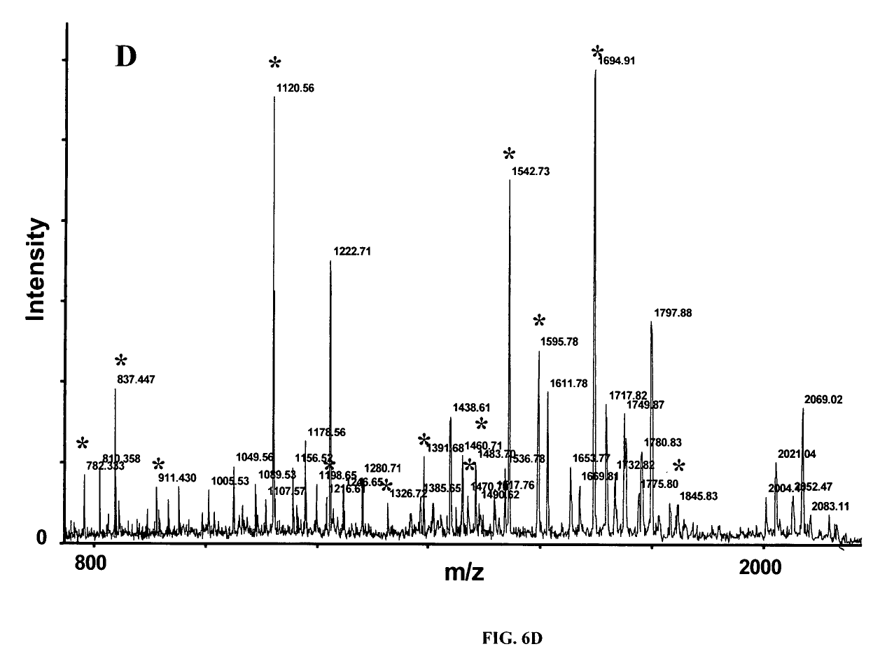

As can be seen from the table, all of CYPs have several isozyme-specific unique tryptic peptides. Not all of these peptides will show up in a tryptic digest and/or produce strong signal in the MALDI TOF mass spectrum. Thus, unique tryptic peptides that consistently formed during the trypsinolysis and generated strongest MS signal were identified. To this end, all three purified isozymes (CYP1A2, CYP2E1, and CYP2C19) were subjected to in-solution tryptic digest (FIG. 6). It should be noted that multiple digests differing in conditions were performed (in-solution vs. in-gel, with or w/o reduction and alkylation, 37° C. overnight vs. 58° C. 45 min) with each of these isozymes, and the results were consistent. For each CYP, there was at least one isozyme-specific unique tryptic peptide that produced a dominant mass peak in the corresponding PMF MALDI TOF mass spectrum (FIG. 6). For CYP1A2, the dominant mass peak occurred with SEQ ID NO. 13 (YLPNPALQR). For CYP2C19, the dominant peak occurred with SEQ. ID NO. 69 (GHMPYTDAVVHEVQR). For CYP 2E1, the dominant peak occurred with SEQ ID NO. 88 (FITLVPSNLPHEATR). The sequences of these peptides were confirmed by MS/MS (data not shown). It should be emphasized that these major isozyme-specific unique tryptic peptides were conserved even in simplified digests performed without destaining of gel bands, reduction and alkylation (cf. panels A and B, FIG. 6).

### EXAMPLE 5

### Quantitation of CYP Isozymes CYP2A2, CYP2E1, and CYP2C19 Using Mass Spectrometry

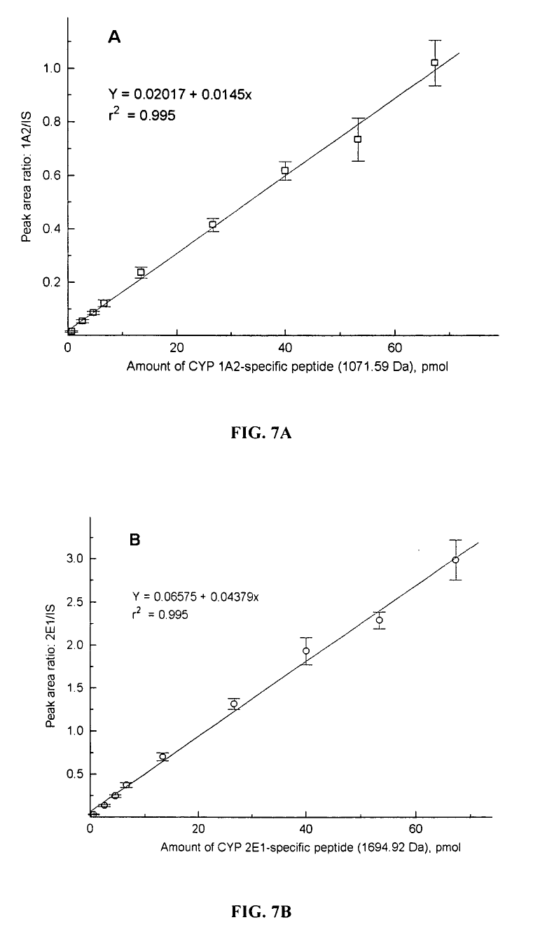

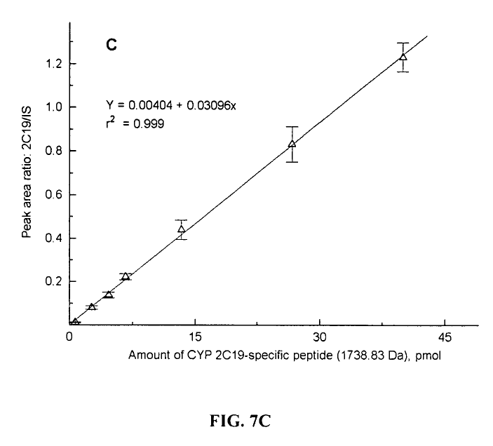

In this example, the identified major isozyme-specific unique tryptic peptides of CYP1A2 (SEQ. ID NO. 13), CYP2E1 ((SEQ. ID NO. 88), and CYP2C19 (SEQ. ID NO. 69) were synthesized and used for quantitative analysis of human CYPs. In the experiment, the CYP2B2 isozyme-specific unique peptide (SEQ. ID NO. 502) was used as an internal standard (“IS”). The calibration curves for the absolute quantitation of CYP isozymes were generated using mixtures of four peptides (internal standard peptide plus three synthetic isozyme-specific unique tryptic peptides). Each MALDI target spot contained 20 pmol of IS peptide and from 500 fmol to 70 pmol of the synthetic CYP1A2 and CYP2E1 isozyme-specific unique tryptic peptides and from 500 fmol to 50 pmol of the synthetic CYP2C19 isozyme-specific tryptic peptide. Linear regression analysis data presented on FIG. 7 indicate that for all three isozymes the peak area ratios are linear with the amount of the synthesized isozyme-specific unique tryptic peptide.

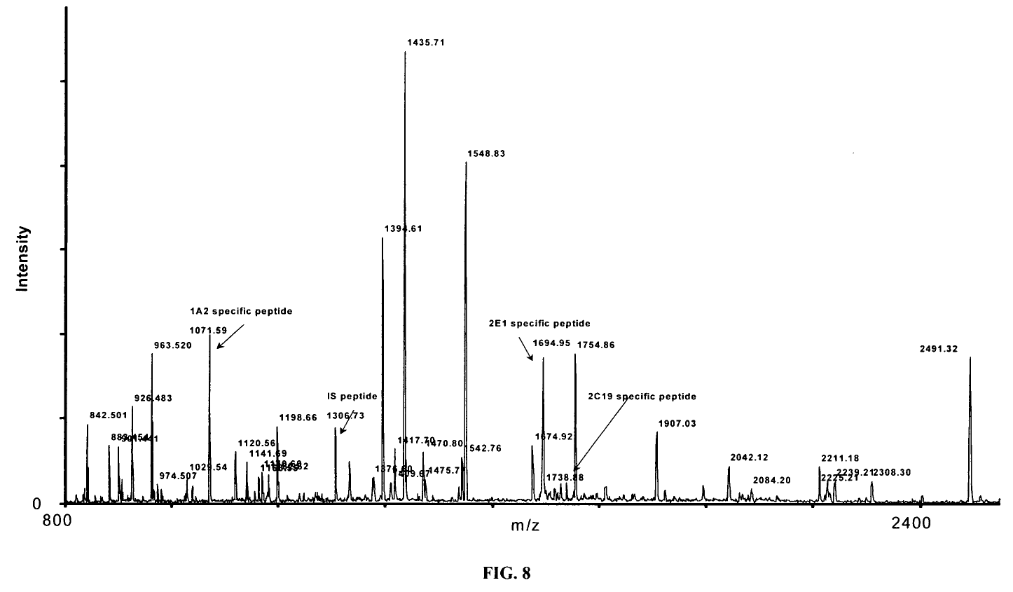

Subsequently, two mixtures of purified CYPs as set forth in the following table were prepared with different molar ratios based on their concentrations determined spectrophotometrically by UV-Vis spectra and then spiked them with IS peptide and performed in-solution tryptic digest. A representative MALDI TOF mass spectrum of a combined digest of all three CYP isozymes is shown in FIG. 8. The peak area ratios of isozyme-specific unique tryptic peptides to IS peptide was measured from MS spectra and the concentrations of all three CYPs in a given mixture were determined simultaneously using the developed calibration curves. The CYP isozymes concentrations measured by MALDI TOF MS were generally higher than the concentrations measured spectrophotometrically in individual CYP stock solutions (table below). Somewhat elevated values of CYP concentrations measured by MALDI TOF MS compared to UV-Vis measurement reflect the fact that the mass spectrometric method measures the apoprotein amount, while UV-Vis measures holoenzyme (CYP molecules containing heme moiety). Since in cytochromes P450 the prosthetic heme group is not covalently bound to the apoprotein (except CYP4As), part of the P450 molecules loses it relatively easily. All three CYP isozymes used in this study were recombinant proteins produced from over-expressed plasmid in E. coli. According to the manufacturer's certification (Invitrogen) their specific content varied from 10 nmol of spectral P450/mg of protein in case of CYP2C19 to 12 nmol/mg protein for CYP2E1, and 16 nmol/mg for CYP1A2. These values indicate presence of heme-depleted CYPs and/or existence of some protein impurity. And, indeed, the proteomic analysis identified presence of β-galactosidase from E. coli in all preparations of these isozymes (data not shown). In line with these findings, CYP concentrations calculated based on protein measurements in CYP isozymes stock solutions were consistently higher than MALDI TOF MS measured values (table below). Noteworthy also is the fact that the higher the P450 specific content (e.g., CYP purity) is, the better is the correlation between MALDI TOF MS and UV-Vis measured values (table below, CYP1A2 vs. CYP2E1 vs. CYP2C19).

| | Concentration of CYP | | | Concentration of CYP | | | |
|---|---|---|---|---|---|---|---|
| | isozymes in mixture 1, | | | isozymes in mixture 2, | | | CYP isozyme specific |
| CYP | pmol/ml | | | pmol/ml | | | content, nmol |
| isozyme | MALDI | UV-VIS | BCA | MALDI | UV-VIS | BCA | P450/mg protein |
| CYP1A2 | 7.6 ± 1.6 | 6.7 | 8.4 | 1.8 ± 0.5 | 2 | 2.6 | 16.3 |
| CYP2E1 | 7.8 ± 1.1 | 5.7 | 8.7 | 11.5 ± 1.5 | 8.1 | 12.3 | 12.3 |
| CYP2C19 | 5.3 ± 1.0 | 3.2 | 6.6 | 8.5 ± 0.3 | 4.6 | 10.5 | 10 |

Due to the low sequence similarity among the predicted isozyme-specific unique tryptic peptides, a labeled IS peptide was not designed, but rather it was decided to use CYP2B2 isozyme-specific unique tryptic peptide (1305.7 Da) as the universal internal standard. However, it should be pointed out, that isozyme-specific unique tryptic peptides for CYP2C19 and CYP2E1 originate from the same part of the CYP molecule as CYP2B1 and CYP2B2 isozyme-specific unique tryptic peptides, while CYP1 A2 isozyme-specific unique tryptic peptide comes from a different part of the molecule. If this trend can be confirmed and extended, then a single internal standard per CYP family/subfamily could be designed what in turn might increase accuracy of this approach.

### EXAMPLE 6

### Antibodies Against Isozyme-Specific Unique Proteolytic Peptides

In this example, it was demonstrated that anti-peptide antibodies raised against isozyme-specific unique tryptic peptides can be utilized for immunochemical (Western and/or ELISA) identification of CYP isozymes. In this example, the CYP2E1 unique tryptic peptide (SEQ. ID NO. 88: FITLVPSNLPHEATR, 1693.915 Da) was used previously in quantitative PMF experiments. The peptide was synthesized on an ACT 90 (Advanced ChemTech, Louisville, Ky.) by means of solid phase technique using Fmoc-protected amino acids. The peptide was purified by semi-preparative HPLC performed on a Summit HPLC system (Dionex, Calif.). The final peptide preparation was analyzed by MALDI-TOF MS and analytical reverse-phase HPLC, and were >99% pure. For the purpose of coupling to the carrier molecule, keyhole limpet haemocyanin (“KLH”), and to a resin to synthesize an affinity resin to be used in affinity purification, a cysteine residue was added to the N-terminus of the peptide. Polyclonal antibodies were raised against peptide-KLH conjugates in New Zealand White rabbits. Ten-week protocol to produce antibodies in two rabbits with the following was used.

| | |
|---|---|
| Day 1 | Bleed 25 mL (yields 10 mL pre-immune serum). 1^{st} Immunize |
| | with antigen in Complete Freund's Adjuvant (CFA). |
| Day 20 | 2^{nd} Immunize with antigen in incomplete Freund's Adjuvant |
| | (IFA). |
| Day 40 | 3^{rd} Immunize with antigen in IFA. |
| Day 50 | Test Bleed 10 mL for ELISA screen(internal quality control |
| | only, not provide to client). |
| Day 60 | 4^{th} Immunize with antigen in IFA. |
| Day 70 | Final Total Bleed 50 mL from Each Rabbit. |

The obtained serum was loaded on the peptide affinity column and eluted with increasing concentration of KSCN. The resultant polyclonal monospecific antibody was used in Example 7.

### EXAMPLE 7

### Immunochemistry Using Antibodies Against Unique Proteolytic Peptide from CYP2E1

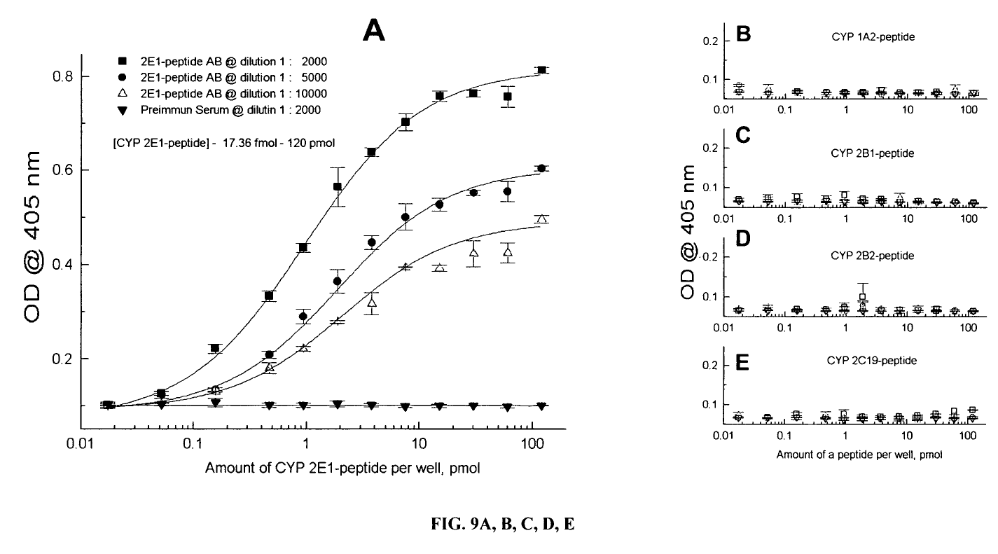

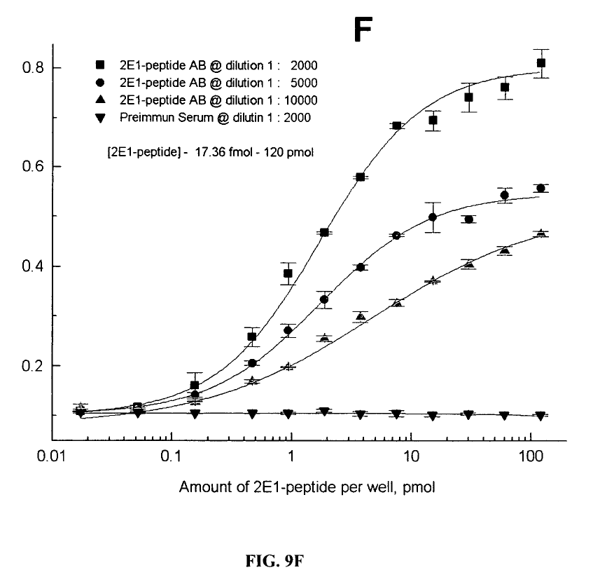

In this example, ELISA was used to investigate the immunoreactivity of the obtained monospecific antibodies toward CYP2E1 peptide (SEQ. ID NO. 88) introduced in the mixture of other peptides, thus mimicking a tryptic digest (FIG. 9, panel F). The antibody has an ELISA titer greater than 100000. The obtained antibody was evaluated by ELISA and Dot-blot. The reaction of developed msAb was positive and highly specific to CYP2E1 unique tryptic peptide as determined by ELISA (FIG. 9) and immunoblot (FIG. 10), and showed no cross-reactivity with tryptic peptides uniquely specific to CYP2B1 (SEQ. ID NO. 501: FSDLVPIGVPHR, 1335.730 Da), CYP2B2 (SEQ. ID NO. 502: FADLAPIGLPHR, 1305.719 Da), CYP1A2 (SEQ. ID NO. 13: YLPNPALQR, 1070.587 Da) and CYP2C19 (SEQ. ID NO. 69 GHMPYTDAVVHEVQR, 1737.826 Da) (FIG. 9 B-E). Comparison of panels A and F on FIG. 9 shows that there was essentially no difference between magnitudes of immunoresponse in both instances.

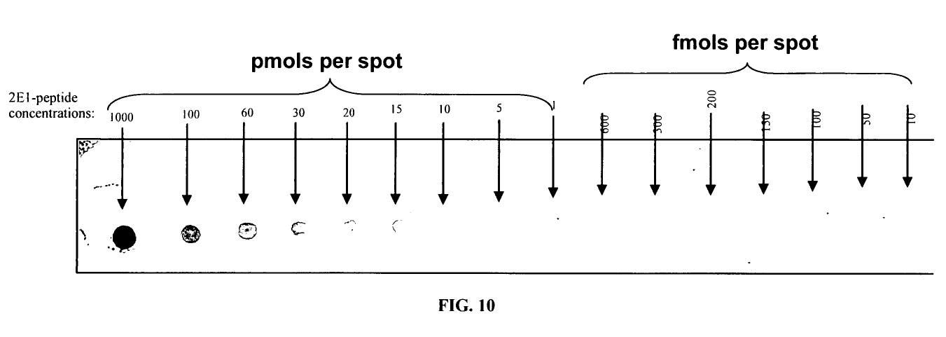

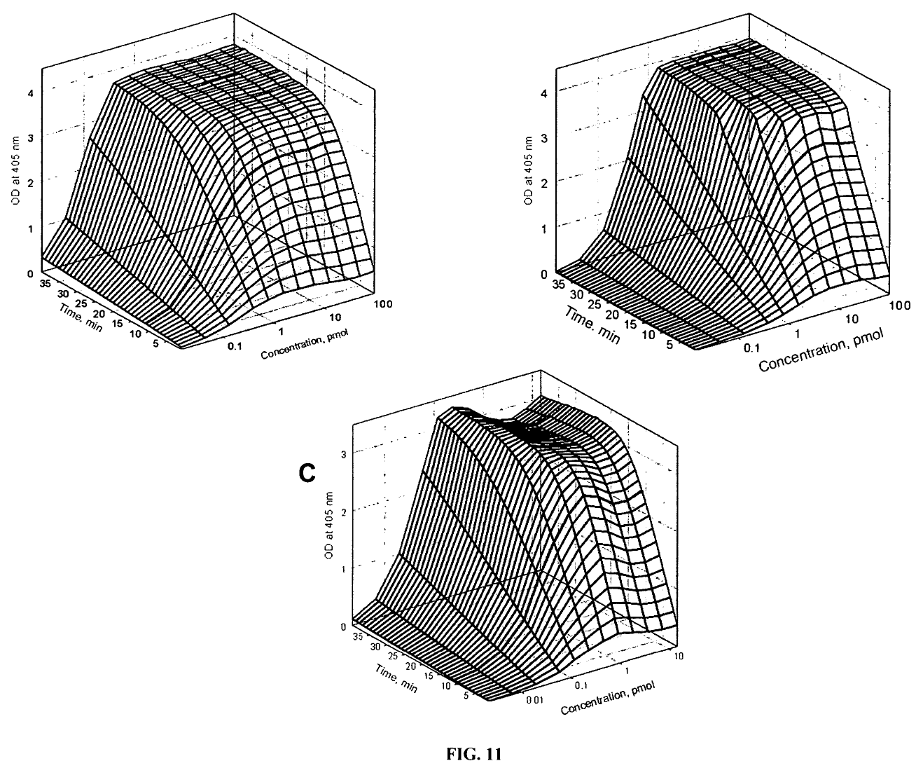

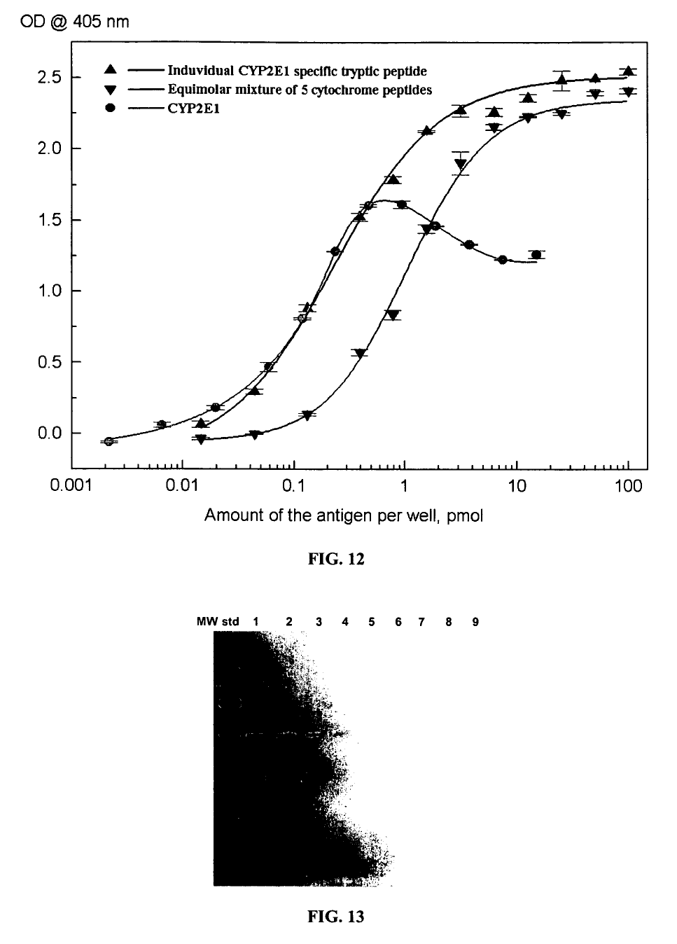

Next, conditions for ELISA experiments were optimized. FIG. 11 shows the relationship between incubation time, concentration of antigen, and optical density. The experiments described above were then repeated under optimal conditions (FIG. 12). The data presented on FIG. 12 clearly demonstrate that ELISA response of peptide-antibody interaction (triangles) and the whole protein-Ab interaction (upside down triangles) overlap on the linear part of the calibration curve, indicating that the amount of the tryptic peptide corresponds to the amount of the whole protein.

In short, the developed antibody demonstrated high affinity and specificity against the whole protein molecule as is seen from FIG. 12. ELISA analysis performed with CYP2E1 protein in the range from 2 fmol to 15 pmol confirmed Western blot data. Both techniques showed no cross reactivity with several human and rat CYP isozymes (CYP2B1, CYP2B2, CYP2A6, CYP2A13, CYP2C19, and CYP2A1, FIG. 13).

### EXAMPLE 7

### Inhibitory Antibodies Against Unique Proteolytic Peptides

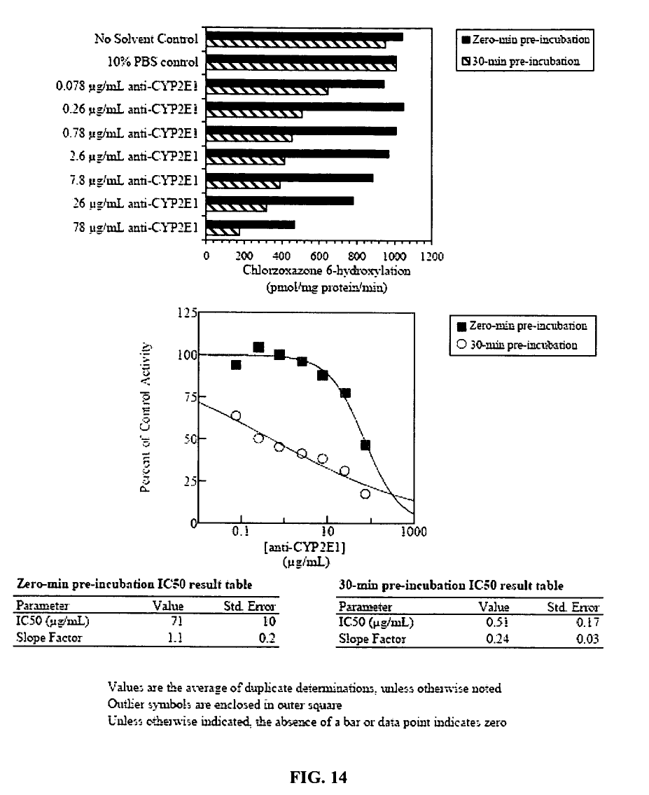

In this example, it was demonstrated that the developed antibodies against the unique tryptic peptide for CYP2E1 also possess an inhibitory potency against CYP2E1 specific activity. Chloraxozone 6-hydroxylation is considered as highly specific (selective) metabolic reaction used for CYP2E1 characterization and correspondingly it was used in this example. The main result is that CYP2E1 isozyme-specific unique tryptic peptide antibody is inhibitory, with an IC50 of 71 μg/mL. About 0.78 mg antibody to 1 mg microsomal protein caused 54% inhibition of chloroxazone 6-hydroxylation when pre-incubated for 15 minutes at room temperature, but 83% inhibition when pre-incubated for an additional 30 minutes at 37 degrees (FIG. 14). All developed up to date monoclonal or polyclonal antibodies against CYP2E1 are either inhibitory or could be used for recognition. Thus, the antibodies of the present invention combine inhibitory and recognition properties.

### EXAMPLE 8

### Immunochemistry Using Antibodies Against Unique Proteolytic Peptide from CYP1A2

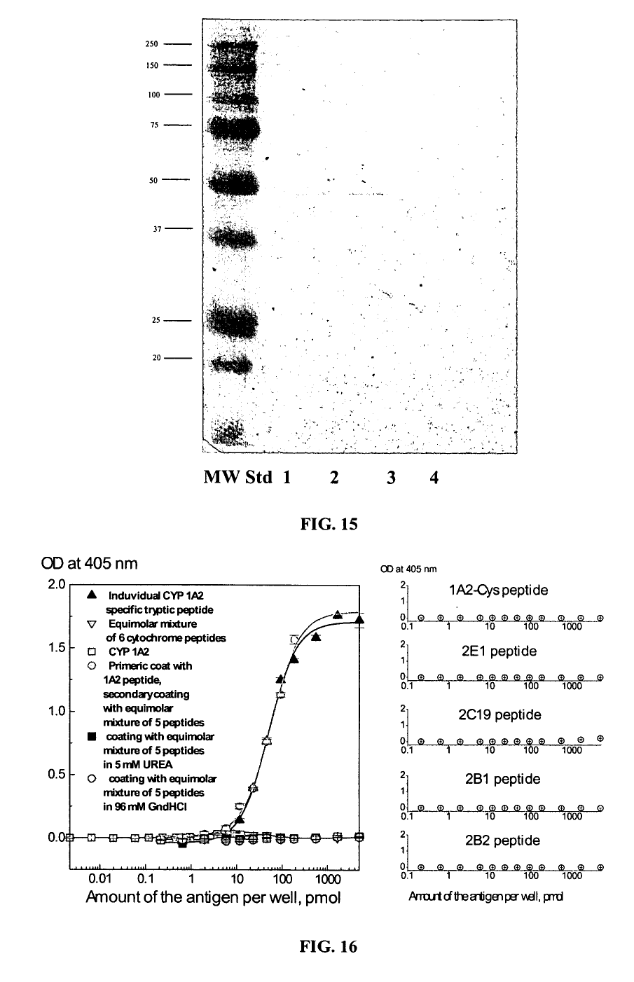

In this example, a antibody against human CYP1A2 isozyme, a major isozyme involved in carcinogenesis, was developed using the same techniques as set forth in Example 6. As in case with CYP2E1, the same CYP1A2 unique tryptic peptide (SEQ. ID NO. 13: YLPNPALQR, 1070.587 Da) used previously in quantitative PMF experiments. As is seen from the Western blot in FIG. 15, the antibodies react with the whole protein. In addition, the developed antibody demonstrated high affinity and specificity against the CYP1A2 isozyme-specific unique tryptic peptide (FIG. 16).

### EXAMPLE 9

### Immunochemistry Using Antibodies Against Unique Proteolytic Peptide from CYP1A2 or CYP2E1 Attached to Magnetic Beads

In this example, the polyclonal mono specific antibodies to CYP2E1 (Example 6) and CYP1A2 (Example 8) were used to extract corresponding peptides from their solutions and determine their concentration. A 40 μL aliquot of commercially available magnetic beads suspension (Magna Bind Protein A and Protein G; Pierce, cat ## 21348 and 21349, correspondingly) were washed twice with PBS and mixed with a 20 μL aliquot of either 1A2-peptide AB, or 2E1-peptide AB with addition of 30 μL of PBS. The mixture was incubated with occasional vortexing for 1 hour at incumbent temperature. The beads were separated from the mixture on a magnetic stand, and washed twice with PBS. A 100 μL aliquot of freshly prepared 30 mM dimethyl pimelimidate in 200 mM thriethanolamine was added to the beads with bound antibodies to perform a covalent cross-linking reaction. After 30 minute of incubation at incumbent temperature, the beads were separated from the cross-linker solution and 500 μL of 150 mM monoethanolamine (pH 9.0) were added to the pelleted beads to quench the reaction. The beads were incubated with quenching solution for another hour and then pelleted and washed twice with PBS. The beads with linked antibodies were either used for experiments immediately after preparation, or stored at +4° C. in PBS buffer, containing traces of sodium azide. In the last case, beads were washed with PBS twice before being used in the assays. According to the company's description, 40 μL of the beads have binding capacity of about 10 μg, which corresponds to about 70 pmol of antibodies. In the preparation of this example, the beads were incubated with 5.2 μg of antibodies in 20 μL aliquot, which corresponds to about 36 pmol.

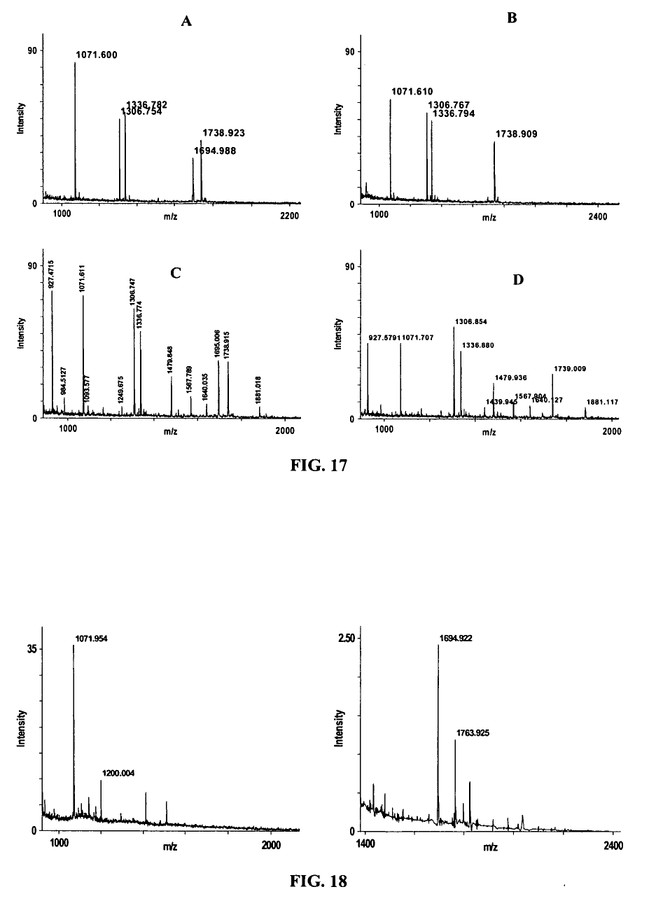

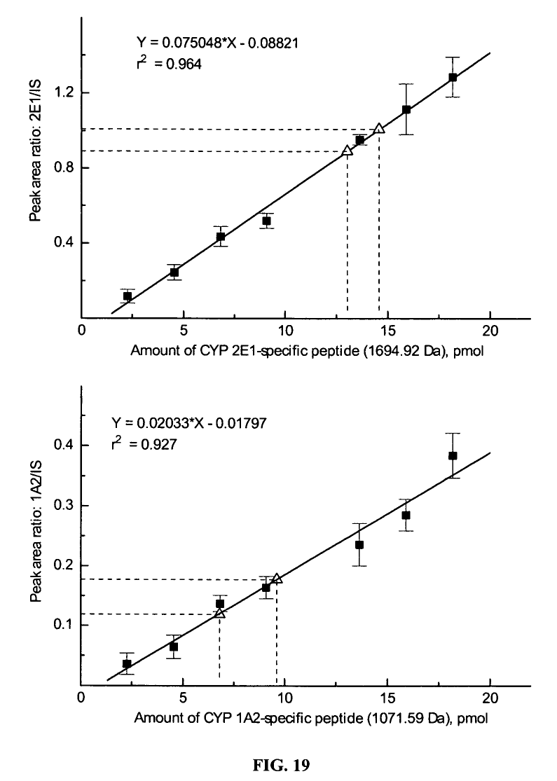

As is seen from the FIG. 17, antibodies bound to Protein G magnetic beads extracted only corresponding isozyme-specific unique tryptic peptides that were later eluted by 1% TFA and analyzed by MALDI TOF MS (FIG. 18). Corresponding calibration curves were created using the same internal standard as in previously described MS experiments and concentrations of CYP2E1 and CYP1A2 determined in solutions (FIG. 19 and following table).

| | Theoretical amount, | Measured amount, |
|---|---|---|
| | pmol per 20 μL aliquot | pmol per 20 μL aliquot |
| CYP1A2 | 10.115 | 8.20481 ± 1.41231 |
| CYP2E1 | 12.24 | 13.8015 ± 0.779 |

### REFERENCES

The following references, to the extent that they provide exemplary procedural or other details supplementary to those set forth herein, are specifically incorporated herein by reference:
Aebersold, R., B. Rist, et al. (2000). “Quantitative Proteome Analysis: Methods and Applications.” Ann NY Acad Sci 919(1): 33-47.
Anderson, L. and J. Seilhamer (1997). “A comparison of selected mRNA and protein abundances in human liver.” Electrophoresis 18(3-4): 533-7.
Anderson, N. L., A. D. Matheson, et al. (2000). “Proteomics: applications in basic and applied biology.” Current Opinion in Biotechnology 11(4): 408-12.
Bakhtiar, R. and R. W. Nelson (2001). “Mass Spectrometry of the Proteome.” Mol Pharmacol 60(3): 405-415.
Baldwin, M. A. (2004). “Protein Identification by Mass Spectrometry: Issues to be Considered.” Mol Cell Proteomics 3(1): 1-9.
Blackstock, W. P. and M. P. Weir (1999). “Proteomics: quantitative and physical mapping of cellular proteins.” Trends in Biotechnology 17(3): 121-7.
Bonk, T. and A. Humeny (2001). “MALDI-TOF-MS analysis of protein and DNA.” Neuroscientist 7(1): 6-12.
Bourrie, M., V. Meunier, et al. (1996). “Cytochrome P450 isoform inhibitors as a tool for the investigation of metabolic reactions catalyzed by human liver microsomes.” J Pharmacol Exp Ther 277(1): 321-332.
Bucknall, M., K. Fung, et al. (2002). “Practical quantitative biomedical applications of MALDI-TOF mass spectrometry.” J Am Soc Mass Spectrom 13(9): 1015-27.
Burke, M. D. and R. T. Mayer (1983). “Differential effects of phenobarbitone and 3-methylcholanthrene induction on the hepatic microsomal metabolism and cytochrome P-450-binding of phenoxazone and a homologous series of its n-alkyl ethers (alkoxyresorufins).” Chem.-Biol. Interactions 45: 243-258.
Chen, G., T. G. Gharib, et al. (2002). “Discordant Protein and mRNA Expression in Lung Adenocarcinomas.” Mol Cell Proteomics 1(4): 304-313.
Chow, T., S. Imaoka, et al. (1999). “Developmental Changes in the Catalytic Activity and Expression of CYP2D Isoforms in the Rat Liver.” Drug Metab Dispos 27(2): 188-192.
Cohen, S. and B. Chait (1996). “Influence of matrix solution conditions on the MALDI-MS analysis of peptides and proteins.” Anal Chem 68(1): 31-7.
Czerwinski, M., T. L. McLemore, et al. (1994). “Quantification of CYP2B7, CYP4B1, and CYPOR messenger RNAs in normal human lung and lung tumors.” Cancer Res 54(4): 1085-91.
Danielson, P. (2002). “The cytochrome P450 superfamily: biochemistry, evolution and drug metabolism in humans.” Curr Drug Metab 3(6): 561-97.
Dierks, E. A., K. R. Stams, et al. (2001). “A Method for the Simultaneous Evaluation of the Activities of Seven Major Human Drug-Metabolizing Cytochrome P450s Using an in Vitro Cocktail of Probe Substrates and Fast Gradient Liquid Chromatography Tandem Mass Spectrometry.” Drug Metab Dispos 29(1): 23-29.
Eagling, V. A., J. F. Tjia, et al. (1998). “Differential selectivity of cytochrome P450 inhibitors against probe substrates in human and rat liver microsomes.” Br J Clin Pharmacol 45(2): 107-114.
Edwards, R. J., A. R. Boobis, et al. (2003). “A strategy for investigating the CYP superfamily using targeted antibodies is a paradigm for functional genomic studies.” Drug Metab Dispos 31(12): 1476-1480.
Figueroa, I., O. Torres, et al. (1998). “Effects of the water content in the sample preparation for MALDI on the mass spectra.” Anal Chem 70(21): 4527-33.
Galeva, N. and M. Altermann (2002). “Comparison of one-dimensional and two-dimensional gel electrophoresis as a separation tool for proteomic analysis of rat liver microsomes: cytochromes P450 and other membrane proteins.” Proteomics 2(6): 713-22.
Galeva, N., D. Yakovlev, et al. (2003). “Direct Identification of Cytochrome P450 Isozymes by Matrix-assisted Laser Desorption/Ionization Time of Flight-Based Proteomic Approach.” Drug Metab Dispos 31(4): 351-355.
Gallagher, E. P., T. M. Buetler, et al. (1995). “The effects of diquat and ciprofibrate on mRNA expression and catalytic activities of hepatic xenobiotic metabolizing and antioxidant enzymes in rat liver.” Toxicol Appl Pharmacol 134(1): 81-91.
Garden, R. and J. Sweedler (2000). “Heterogeneity within MALDI samples as revealed by mass spectrometric imaging.” Anal Chem 72(1): 30-6.
Gelboin, H., I. Goldfarb, et al. (1996). “Inhibitory and noninhibitory monoclonal antibodies to human cytochrome P450 2E1.” Chem Res Toxicol 9(6): 1023-30.
Gelboin, H., K. Krausz, et al. (1995). “Inhibitory and non-inhibitory monoclonal antibodies to human cytochrome P450 3A3/4.” Biochem Pharmacol 50(11): 1841-50.
Gerber, S. A., J. Rush, et al. (2003). “Absolute quantification of proteins and phosphoproteins from cell lysates by tandem MS.” PNAS 100(12): 6940-6945.
Guengerich, F. P. (1995). “Cytochrome P450 proteins and potential utilization in biodegradation.” Environ Health Perspect 103 Suppl 5: 25-8.
Guengerich, F. P. (1997). “Comparisons of catalytic selectivity of cytochrome P450 subfamily enzymes from different species.” Chem Biol Interact 106(3): 161-82.
Guengerich, F. P. (2003). “Cytochromes P450, Drugs, and Diseases.” Mol. Interv. 3(4): 194-204.
Gygi, S. P. and R. Aebersold (2000). “Mass spectrometry and proteomics.” Curr Opin Chem Biol 4(5): 489-94.
Helmke, S., C. Yen, et al. (2004). “Simultaneous quantification of human cardiac alpha- and beta-myosin heavy chain proteins by MALDI-TOF mass spectrometry.” Anal Chem 76(6): 1683-9.
Hlongwane, C., I. Delves, et al. (2001). “Comparative quantitative fatty acid analysis of triacylglycerols using matrix-assisted laser desorption/ionization time-of-flight mass spectrometry and gas chromatography.” Rapid Commun Mass Spectrom 15(21): 2027-34.
Huber, M., I. Bahr, et al. (2004). “Comparison of Proteomic and Genomic Analyses of the Human Breast Cancer Cell Line T47D and the Antiestrogen-resistant Derivative T47D-r.” Mol Cell Proteomics 3(1): 43-55.
Hunter, T., N. Andon, et al. (2002). “The functional proteomics toolbox: methods and applications.” J Chromatogr B Analyt Technol Biomed Life Sci 782(1-2): 165-81.
Jung, E., M. Heller, et al. (2000). “Proteomics meets cell biology: the establishment of subcellular proteomes.” Electrophoresis 21(16): 3369-77.
Knochenmuss, R., F. Dubois, et al. (1999). “The Matrix Suppression Effect and Ionization Mechanisms in Matrix-assisted Laser Desorption/Ionization.” Rapid Commun Mass Spectrom 10(8): 871-77.
Kobayashi, K., K. Urashima, et al. (2002). “Substrate specificity for rat cytochrome P450 (CYP) isoforms: screening with cDNA-expressed systems of the rat.” Biochem Pharmacol 63(5): 889-96.
Kobayashi, K., K. Urashima, et al. (2003). “Selectivities of human cytochrome P450 inhibitors toward rat P450 isoforms: study with cDNA-expressed systems of the rat.” Drug Metab Dispos 31(7): 833-836.
Kobayashi, S., M. Nakano, et al. (1987). “On the Mechanism of the Peroxidase-Catalyzed Oxygen-Transfer Reaction.” biochem 26: 5019-5022.
Kratzer, R., C. Eckerskorn, et al. (1998). “Suppression effects in enzymatic peptide ladder sequencing using ultraviolet—matrix assisted laser desorption/ionization—mass spectormetry.” Electrophoresis 19(11): 1910-9.
Krausz, K. W., I. Goldfarb, et al. (2001). “Monoclonal Antibodies Specific and Inhibitory to Human Cytochromes P450 2C8, 2C9, and 2C19.” Drug Metab Dispos 29(11): 1410-1423.
Lewis, D. (2003). “P450 structures and oxidative metabolism of xenobiotics.” Pharmacogenomics 4(4): 387-95.
Lill, J. (2003). “Proteomic tools for quantitation by mass spectrometry.” Mass Spectrom Rev 22(3): 182-94.
Lin, J. H. (1998). “Applications and Limitations of Interspecies Scaling and In Vitro Extrapolation in Pharmacokinetics.” Drug Metab Dispos 26(12): 1202-1212.
Luss, H., R. Li, et al. (1997). “Dedifferentiated human ventricular cardiac myocytes express inducible nitric oxide synthase mRNA but not protein in response to IL-1, TNF, IFNgamma, and LPS.” J Mol Cell Cardiol 29(4): 1153-65.
Mann, M., R. C. Hendrickson, et al. (2001). “ANALYSIS OF PROTEINS AND PROTEOMES BY MASS SPECTROMETRY.” Annu. Rev. Biochem. 70(1): 437-473.
McFadyen, M. C. E., W. T. Melvin, et al. (2004). “Cytochrome P450 enzymes: Novel options for cancer therapeutics.” Mol Cancer Ther 3(3): 363-371.
Mirgorodskaya, O., Y. Kozmin, et al. (2000). “Quantitation of peptides and proteins by matrix-assisted laser desorption/ionization mass spectrometry using (18)O-labeled internal standards.” Rapid Commun Mass Spectrom 14(14): 1226-32.
Moritz, B. and H. Meyer (2003). “Approaches for the quantification of protein concentration ratios.” Proteomics 3(11): 2208-20.
Nelson, D. (1998). “Metazoan cytochrome P450 evolution.” Comp Biochem Physiol C Pharmacol Toxicol Endocrinol 121(1-3): 15-22.
Newsholme, S. J., B. F. Maleeff, et al. (2000). “Two-dimensional electrophoresis of liver proteins: characterization of a drug-induced hepatomegaly in rats.” Electrophoresis 21(11): 2122-8.
Newton, D., R. Wang, et al. (1995). “Cytochrome P450 inhibitors. Evaluation of specificities in the in vitrometabolism of therapeutic agents by human liver microsomes.” Drug Metab Dispos 23(1): 154-158.
Nisar, S., C. S. Lane, et al. (2004). “A proteomic approach to the identification of cytochrome P450 isoforms in male and female rat liver by nanoscale liquid chromatography-electrospray ionization-tandem mass spectrometry.” Drug Metab Dispos 32(4): 382-386.
Omiecinski, C. J., C. A. Redlich, et al. (1990). “Induction and developmental expression of cytochrome P4501A1 messenger RNA in rat and human tissues: detection by the polymerase chain reaction.” Cancer Res 50(14): 4315-21.
Ong, S.-E., B. Blagoev, et al. (2002). “Stable Isotope Labeling by Amino Acids in Cell Culture, SILAC, as a Simple and Accurate Approach to Expression Proteomics.” Mol Cell Proteomics 1(5): 376-386.
Patterson, S. D. (2000). “Mass spectrometry and proteomics.” Physiol Genomics 2(2): 59-65.
Patterson, S. D. (2000). “Proteomics: the industrialization of protein chemistry.” Current Opinion in Biotechnology 11(4): 413-8.
Poulos, T. L. (1995). “Cytochrome P450.” Curr Opin Struct Biol 5(6): 767-74.
Rochat, B., E. M. J. Gillam, et al. (2003). “Evaluation of Recombinant Cytochromes P450 Activity in Metabolic Pathways.” Drug Metab Dispos 31(1): 145-146.
Salonen, J. S., L. Nyman, et al. (2003). “Comparative studies on the cytochrome P450-associated metabolism and interaction potential of selegiline between human liver-derived in vitro systems.” Drug Metab Dispos 31(9): 1093-1102.
Schenkman, J. B., A. I. Voznesensky, et al. (1994). “Influence of ionic strength on the P450 monooxygenase reaction and role of cytochrome b5 in the process.” Arch Biochem Biophys 314(1): 234-41.
Sechi, S. and Y. Oda (2003). “Quantitative proteomics using mass spectrometry.” Curr Opin Chem Biol 7(1): 70-7.
Shet, M. S., K. M. Faulkner, et al. (1995). “The effects of cytochrome b5, NADPH-P450 reductase, and lipid on the rate of 6 beta-hydroxylation of testosterone as catalyzed by a human P450 3A4 fusion protein.” Arch Biochem Biophys 318(2): 314-21.
Shou, M., T. Lu, et al. (2000). “Use of inhibitory monoclonal antibodies to assess the contribution of cytochromes P450 to human drug metabolism.” Eur J Pharmacol 394(2-3): 199-209.
Soars, M., H. Gelboin, et al. (2003). “A comparison of relative abundance, activity factor and inhibitory monoclonal antibody approaches in the characterization of human CYP enzymology.” Br J Clin Pharmacol 55(2): 175-81.
Stresser, D. M., S. D. Turner, et al. (2002). “Cytochrome P450 Fluorometric Substrates: Identification of Isoform-Selective Probes for Rat CYP2D2 and Human CYP3A4.” Drug Metab Dispos 30(7): 845-852.
Tran, T. H., L. L. von Moltke, et al. (2002). “Microsomal Protein Concentration Modifies the Apparent Inhibitory Potency of CYP3A Inhibitors.” Drug Metab Dispos 30(12): 1441-1445.
Vanden Heuvel, J. P., G. C. Clark, et al. (1993). “CYP1A1 mRNA levels as a human exposure biomarker: use of quantitative polymerase chain reaction to measure CYP1A1 expression in human peripheral blood lymphocytes.” Carcinogenesis 14(10): 2003-6.
Venkatakrishnan, K., L. L. von Moltke, et al. (2000). “Comparison between Cytochrome P450 (CYP) Content and Relative Activity Approaches to Scaling from cDNA-Expressed CYPs to Human Liver Microsomes: Ratios of Accessory Proteins as Sources of Discrepancies between the Approaches.” Drug Metab Dispos 28(12): 1493-1504.
Venkatakrishnan, K., L. L. von Moltke, et al. (2001). “Application of the Relative Activity Factor Approach in Scaling from Heterologously Expressed Cytochromes P450 to Human Liver Microsomes: Studies on Amitriptyline as a Model Substrate.” J Pharmacol Exp Ther 297(1): 326-337.
Walker, A., C. Land, et al. (2000). “Quantitative determination of the peptide retention of polymeric substrates using matrix-assisted laser desorption/ionization mass spectrometry.” J Am Soc Mass Spectrom 11(1): 62-8.
Washburn, M., R. Ulaszek, et al. (2002). “Analysis of quantitative proteomic data generated via multidimensional protein identification technology.” Anal Chem 74(7): 1650-7.
Weaver, R., K. S. Graham, et al. (2003). “Cytochrome P450 inhibition using recombinant proteins and mass spectrometry/multiple reaction monitoring technology in a cassette incubation.” Drug Metab Dispos 31(7): 955-966.
Weston, A. and L. Hood (2004). “Systems biology, proteomics, and the future of health care: toward predictive, preventative, and personalized medicine.” J Proteome Res 3(2): 179-96.
Whetstone, P., N. Butlin, et al. (2004). “Element-coded affinity tags for peptides and proteins.” Bioconjug Chem 15(1): 3-6.
Yan, W., H. Lee, et al. (2004). “A Dataset of Human Liver Proteins Identified by Protein Profiling Via Isotope-coded Affinity Tag (ICAT) and Tandem Mass Spectrometry.” Mol Cell Proteomics 3(10): 1039-1041.
Yao, X., A. Freas, et al. (2001). “Proteolytic 180 labeling for comparative proteomics: model studies with two serotypes of adenovirus.” Analytical Chemistry 73(13): 2836-42.
Yates, J. R. (2000). “Mass spectrometry. From genomics to proteomics.” Trends in Genetics 16(1): 5-8.
Yu, L. J., J. Matias, et al. (2001). “P450 Enzyme Expression Patterns in the NCI Human Tumor Cell Line Panel.” Drug Metab Dispos 29(3): 304-312.
Zappacosta, F. and R. Annan (2004). “N-terminal isotope tagging strategy for quantitative proteomics: results-driven analysis of protein abundance changes.” Anal Chem 76(22): 6618-27.
Zhang, H., W. Yan, et al. (2004). “Chemical probes and tandem mass spectrometry: a strategy for the quantitative analysis of proteomes and subproteomes.” Curr Opin Chem Biol 8(1): 66-75.
Zhang, Q.-Y., D. Dunbar, et al. (1999). “Characterization of Human Small Intestinal Cytochromes P-450.” Drug Metab Dispos 27(7): 804-809.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying figures are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. A method for detecting a protein of interest in a sample comprising:
obtaining a sample;
identifying a unique proteolytic peptide derived from the protein of interest by digestion with a protease;
subjecting the sample to proteolysis using said protease to obtain a mixture of proteolytic peptides;
detecting the unique proteolytic peptide in said mixture;
wherein the presence or absence of said unique proteolytic peptide in said mixture is indicative of the presence or absence of said protein of interest in said sample.

2. The method of claim 1 wherein the protein of interest is a member of the P450 superfamily.

3. The method of claim 1 wherein said sample is a mammalian sample.

4. The method of claim 1 wherein said protease is trypsin.

5. The method of claim 1 wherein said detecting step is performed by detecting the unique proteolytic peptide using mass spectrometry.

6. The method of claim 5 wherein said mass spectrometry is matrix-assisted laser dissorption/ionization time of flight (MALDI-TOF) mass spectrometry.

7. The method of claim 1 wherein said protein of interest is a member of the cytochrome P450 family.

8. The method of claim 1 wherein said step of identifying a unique proteolytic peptide derived from the protein of interest by digestion with a protease is performed using the SwisProt or NCBI database to a generate simulated tryptic digest followed by a comparative analysis with simulated tryptic digests with all proteins in the SwissProt or NCBI database.

9. The method of claim 1 wherein said unique proteolytic peptide has a mass between 900 and 1900 Da.

10. The method of claim 1 wherein said unique proteolytic peptide has an arginine residue at the C-terminus.

11. The method of claim 1 wherein said unique proteolytic peptide is a unique tryptic peptide selected from the group consisting of SEQ ID NO. 1 to 502.

12. The method of claim 1 wherein said protein of interest is an isozyme of the cytochrome P450 family, and wherein said unique proteolytic peptide is a unique tryptic peptide for said isozyme of the cytochrome P450 family, and wherein said protease is trypsin, and wherein said detecting step is performed using MALDI-TOF MS.

13. The method of claim 1 wherein said protein of interest is an isozyme of the cytochrome P450 family, and wherein said unique proteolytic peptide is a unique tryptic peptide for said isozyme of the cytochrome P450 family, and wherein said protease is trypsin, and wherein said detecting step is performed using immunochemistry.

14. The method of claim 13 wherein said immunochemistry is a fluorescent antibody method, enzyme-linked immunosorbent assay method (ELISA), radioimmunoassay (RIA), or sandwich ELISA method.

15. The method of claim 1 wherein said detecting step is performed using both MALDI-TOF MS and immunochemistry.

16. The method of claim 1 wherein further comprising the step of quantifying the amount of unique proteolytic peptide in the mixture.

17. The method of claim 16 wherein the step of quantifying the amount of unique proteolytic peptide is performed using mass spectrometry to generate a mass spectrum.

18. The method of claim 17 wherein the quantifying step is performed by determining a monoisotopic peak area for said unique proteolytic peptide and correlating that area to an amount of peptide using a standard curve.

19. The method of claim 17 further comprising the step of adding an internal standard peptide to said mixture of proteolytic peptides.

20. The method of claim 19 wherein the quantifying step comprises determining the ratio of a monoisotopic peak area for said unique proteolytic peptide to a monoisotopic peak area for said internal standard peptide.

21. The method of claim 16 wherein said the step of quantifying the amount of unique proteolytic peptide is performed using immunochemistry.

22. The method of claim 21 wherein said immunochemistry is a fluorescent antibody method, enzyme-linked immunosorbent assay method (ELISA), radioimmunoassay (RIA), or sandwich ELISA method.

23. A antibody that binds an epitope consisting essentially of a unique tryptic peptide derived from a cytochrome P450 isozyme.

24. The antibody of claim 23 which is a monospecific polyclonal antibody.

25. The antibody of claim 23 wherein said epitope is selected from a unique tryptic peptide having SEQ ID No. 1-502.

26. The antibody of claim 25 which is a monospecific polyclonal antibody.

27. The antibody of claim 23 labeled with a reporter group.

28. The antibody of claim 23 wherein said epitope is a CYP2E1 unique tryptic peptide having SEQ ID NO. 88 (FITLVPSNLPHEATR).

29. The antibody of claim 28 which is a monospecific polyclonal antibody.

30. The antibody of claim 28 wherein said antibody is inhibitory as demonstrated by an assay from chloroxazone 6-hydroxylation.

31. The antibody of claim 23 wherein said epitope is a CYP1A2 unique tryptic peptide having SEQ ID NO. 13 (YLPNPALQR).

32. The antibody of claim 31 which is a monospecific polyclonal antibody.


SEQUENCE LISTING (502 sequences)

SEQ ID NO: 1 - protein, 12 aa, Homo sapiens
  misc_feature (1)..(12): CYP1A1 (Residues 66-77)
MSQQYGDVLQIR

SEQ ID NO: 2 - protein, 11 aa, Homo sapiens
  MISC_FEATURE (1)..(11): CYP1A1 (Residues 242-252)
YLPNPSLNAFK

SEQ ID NO: 3 - protein, 14 aa, Homo sapiens
  MISC_FEATURE (1)..(14): CYP1A1 (Residues 293-306)
QLDENANVQLSDEK

SEQ ID NO: 4 - protein, 11 aa, Homo sapiens
  MISC_FEATURE (1)..(11): CYP1A1 (Residues 343-353)
IQEELDTVIGR

SEQ ID NO: 5 - protein, 15 aa, Homo sapiens
  MISC_FEATURE (1)..(15): CYP1A1 (Residues 363-377)
SHLPYMEAFILETFR

SEQ ID NO: 6 - protein, 15 aa, Homo sapiens
  MISC_FEATURE (1)..(15): CYP1A1 (Residues 378-392)
HSSFVPFTIPHSTTR

SEQ ID NO: 7 - protein, 12 aa, Homo sapiens
  MISC_FEATURE (1)..(12): CYP1A1 (Residues 420-431)
LWVNPSEFLPER

SEQ ID NO: 8 - protein, 10 aa, Homo sapiens
  MISC_FEATURE (1)..(10): CYP1A1 (Residues 432-441)
FLTPDGAIDK

SEQ ID NO: 9 - protein, 13 aa, Homo sapiens
  MISC_FEATURE (1)..(13): CYP1A1 (Residues 465-477)
WEVFLFLAILLQR

SEQ ID NO: 10 - protein, 10 aa, Homo sapiens
  MISC_FEATURE (1)..(10): CYP1A1 (Residues 478-487)
VEFSVPLGVK

SEQ ID NO: 11 - protein, 12 aa, Homo sapiens
  MISC_FEATURE (1)..(12): CYP1A1 (Residues 488-499)
VDMTPIYGLTMK

SEQ ID NO: 12 - protein, 11 aa, Homo sapiens
  MISC_FEATURE (1)..(11): CYP1A2 (Residues 80-90)
IGSTPVLVLSR

SEQ ID NO: 13 - protein, 9 aa, Homo sapiens
  MISC_FEATURE (1)..(9): CYP1A2 (Residues 244-252)
YLPNPALQR

SEQ ID NO: 14 - protein, 11 aa, Homo sapiens
  MISC_FEATURE (1)..(11): CYP1A2 (Residues 267-277)
TVQEHYQDFDK

SEQ ID NO: 15 - protein, 10 aa, Homo sapiens
  MISC_FEATURE (1)..(10): CYP1A2 (Residues 297-306)
ASGNLIPQEK

SEQ ID NO: 16 - protein, 15 aa, Homo sapiens
  MISC_FEATURE (1)..(15): CYP1A2 (Residues 378-392)
HSSFLPFTIPHSTTR

SEQ ID NO: 17 - protein, 11 aa, Homo sapiens
  MISC_FEATURE (1)..(11): CYP1A2 (Residues 393-403)
DTTLNGFYIPK

SEQ ID NO: 18 - protein, 16 aa, Homo sapiens
  MISC_FEATURE (1)..(16): CYP1A2 (Residues 432-447)
FLTADGTAINKPLSEK

SEQ ID NO: 19 - protein, 12 aa, Homo sapiens
  MISC_FEATURE (1)..(12): CYP1A2 (Residues 489-500)
VDLTPIYGLTMK

SEQ ID NO: 20 - protein, 12 aa, Homo sapiens
  MISC_FEATURE (1)..(12): CYP1B1 (Residues 131-142)
SMAFGHYSEHWK

SEQ ID NO: 21 - protein, 12 aa, Homo sapiens
  MISC_FEATURE (1)..(12): CYP1B1 (Residues 164-175)
QVLEGHVLSEAR

SEQ ID NO: 22 - protein, 9 aa, Homo sapiens
  MISC_FEATURE (1)..(9): CYP1B1 (Residues 214-222)
YSHDDPEFR

SEQ ID NO: 23 - protein, 11 aa, Homo sapiens
  MISC_FEATURE (1)..(11): CYP1B1 (Residues 223-233)
ELLSHNEEFGR

SEQ ID NO: 24 - protein, 9 aa, Homo sapiens
  MISC_FEATURE (1)..(9): CYP1B1 (Residues 267-275)
NFSNFILDK

SEQ ID NO: 25 - protein, 12 aa, Homo sapiens
  MISC_FEATURE (1)..(12): CYP1B1 (Residues 291-302)
DMMDAFILSAEK

SEQ ID NO: 26 - protein, 11 aa, Homo sapiens
  MISC_FEATURE (1)..(11): CYP1B1 (Residues 356-366)
VQAELDQVVGR

SEQ ID NO: 27 - protein, 11 aa, Homo sapiens
  MISC_FEATURE (1)..(11): CYP1B1 (Residues 434-444)
WPNPENFDPAR

SEQ ID NO: 28 - protein, 12 aa, Homo sapiens
  MISC_FEATURE (1)..(12): CYP1B1 (Residues 503-514)
MNFSYGLTIKPK

SEQ ID NO: 29 - protein, 16 aa, Homo sapiens
  MISC_FEATURE (1)..(16): CYP1B1 (Residues 524-539)
ESMELLDSAVQNLQAK

SEQ ID NO: 30 - protein, 13 aa, Homo sapiens
  MISC_FEATURE (1)..(13): CYP2A6 (Residues 149-161)
IQEEAGFLIDAHR

SEQ ID NO: 31 - protein, 15 aa, Homo sapiens
  MISC_FEATURE (1)..(15): CYP2A6 (Residues 162-176)
GTGGANIDPTFFLSR

SEQ ID NO: 32 - protein, 13 aa, Homo sapiens
  MISC_FEATURE (1)..(13): CYP2A6 (Residues 388-400)
GTEVYPMLGSVLR

SEQ ID NO: 33 - protein, 13 aa, Homo sapiens
  MISC_FEATURE (1)..(13): CYP2A7 (Residues 149-161)
IQEESGFLIEAIR

SEQ ID NO: 34 - protein, 15 aa, Homo sapiens
  MISC_FEATURE (1)..(15): CYP2A7 (Residues 162-176)
SSHGANIDPTFFLSR

SEQ ID NO: 35 - protein, 13 aa, Homo sapiens
  MISC_FEATURE (1)..(13): CYP2A7 (Residues 388-400)
GTEVFPMLGSVLR

SEQ ID NO: 36 - protein, 11 aa, Homo sapiens
  MISC_FEATURE (1)..(11): CYP2A13 (Residues 102-112)
GEQATFDWLFK

SEQ ID NO: 37 - protein, 13 aa, Homo sapiens
  MISC_FEATURE (1)..(13): CYP2A13 (Residues 149-161)
IQEEAGFLIDALR

SEQ ID NO: 38 - protein, 15 aa, Homo sapiens
  MISC_FEATURE (1)..(15): CYP2A13 (Residues 162-176)
GTHGANIDPTFFLSR

SEQ ID NO: 39 - protein, 11 aa, Homo sapiens
  MISC_FEATURE (1)..(11): CYP2A13 (Residues 240-250)
ELQGLEDFIAK

SEQ ID NO: 40 - protein, 13 aa, Homo sapiens
  MISC_FEATURE (1)..(13): CYP2A13 (Residues 349-361)
MPYTEAVIHEIQR

SEQ ID NO: 41 - protein, 12 aa, Homo sapiens
  MISC_FEATURE (1)..(12): CYP2A13 (Residues 362-373)
FGDMLPMGLAHR

SEQ ID NO: 42 - protein, 13 aa, Homo sapiens
  MISC_FEATURE (1)..(13): CYP2A13 (Residues 388-400)
GTEVFPMLGSELR

SEQ ID NO: 43 - protein, 9 aa, Homo sapiens
  MISC_FEATURE (1)..(9): CYP2B6 (Residues 101-109)
IAMVDPFFR

SEQ ID NO: 44 - protein, 11 aa, Homo sapiens
  MISC_FEATURE (1)..(11): CYP2B6 (Residues 110-120)
GYGVIFANGNR

SEQ ID NO: 45 - protein, 10 aa, Homo sapiens
  MISC_FEATURE (1)..(10): CYP2B6 (Residues 188-197)
FHYQDQEFLK

SEQ ID NO: 46 - protein, 12 aa, Homo sapiens
  MISC_FEATURE (1)..(12): CYP2B6 (Residues 263-274)
DLIDTYLLHMEK

SEQ ID NO: 47 - protein, 13 aa, Homo sapiens
  MISC_FEATURE (1)..(13): CYP2B6 (Residues 346-358)
MPYTEAVIYEIQR

SEQ ID NO: 48 - protein, 11 aa, Homo sapiens
  MISC_FEATURE (1)..(11): CYP2B6 (Residues 423-433)
TEAFIPFSLGK

SEQ ID NO: 49 - protein, 13 aa, Homo sapiens
  MISC_FEATURE (1)..(13): CYP2C8 (Residues 85-97)
EALIDNGEEFSGR

SEQ ID NO: 50 - protein, 9 aa, Homo sapiens
  MISC_FEATURE (1)..(9): CYP2C8 (Residues 191-199)
DQNFLTLMK

SEQ ID NO: 51 - protein, 12 aa, Homo sapiens
  MISC_FEATURE (1)..(12): CYP2C8 (Residues 250-261)
EHQASLDVNNPR

SEQ ID NO: 52 - protein, 11 aa, Homo sapiens
  MISC_FEATURE (1)..(11): CYP2C8 (Residues 323-333)
VQEEIDHVIGR

SEQ ID NO: 53 - protein, 16 aa, Homo sapiens
  MISC_FEATURE (1)..(16): CYP2C8 (Residues 384-399)
GTTIMALLTSVLHDDK

SEQ ID NO: 54 - protein, 8 aa, Homo sapiens
  MISC_FEATURE (1)..(8): CYP2C9 (Residues 98-105)
GIFPLAER

SEQ ID NO: 55 - protein, 16 aa, Homo sapiens
  MISC_FEATURE (1)..(16): CYP2C9 (Residues 384-399)
GTTILISLTSVLHDNK

SEQ ID NO: 56 - protein, 8 aa, Homo sapiens
  MISC_FEATURE (1)..(8): CYP2C10 (Residues 98-105)
GIFPLAER

SEQ ID NO: 57 - protein, 16 aa, Homo sapiens
  MISC_FEATURE (1)..(16): CYP2C10 (Residues 384-399)
GTTILISLTSVLHDNK

SEQ ID NO: 58 - protein, 13 aa, Homo sapiens
  MISC_FEATURE (1)..(13): CYP2C18 (Residues 85-97)
EALIDHGEEFSGR

SEQ ID NO: 59 - protein, 10 aa, Homo sapiens
  MISC_FEATURE (1)..(10): CYP2C18 (Residues 109-118)
GLGILFSNGK

SEQ ID NO: 60 - protein, 9 aa, Homo sapiens
  MISC_FEATURE (1)..(9): CYP2C18 (Residues 233-241)
IAENFAYIK

SEQ ID NO: 61 - protein, 12 aa, Homo sapiens
  MISC_FEATURE (1)..(12): CYP2C18 (Residues 250-261)
EHQESLDMNSAR

SEQ ID NO: 62 - protein, 16 aa, Homo sapiens
  MISC_FEATURE (1)..(16): CYP2C18 (Residues 384-399)
GMTIITSLTSVLHNDK

SEQ ID NO: 63 - protein, 13 aa, Homo sapiens
  MISC_FEATURE (1)..(13): CYP2C18 (Residues 466-478)
DIDITPIANAFGR

SEQ ID NO: 64 - protein, 14 aa, Homo sapiens
  MISC_FEATURE (1)..(14): CYP2C19 (Residues 60-73)
IYGPVFTLYFGLER

SEQ ID NO: 65 - protein, 11 aa, Homo sapiens
  MISC_FEATURE (1)..(11): CYP2C19 (Residues 74-84)
MVVLHGYEVVK

SEQ ID NO: 66 - protein, 8 aa, Homo sapiens
  MISC_FEATURE (1)..(8): CYP2C19 (Residues 98-105)
GHFPLAER

SEQ ID NO: 67 - protein, 12 aa, Homo sapiens
  MISC_FEATURE (1)..(12): CYP2C19 (Residues 236-247)
NLAFMESDILEK

SEQ ID NO: 68 - protein, 12 aa, Homo sapiens
  MISC_FEATURE (1)..(12): CYP2C19 (Residues 250-261)
EHQESMDINNPR

SEQ ID NO: 69 - protein, 15 aa, Homo sapiens
  MISC_FEATURE (1)..(15): CYP2C19 (Residues 343-357)
GHMPYTDAVVHEVQR

SEQ ID NO: 70 - protein, 16 aa, Homo sapiens
  MISC_FEATURE (1)..(16): CYP2C19 (Residues 384-399)
GTTILTSLTSVLHDNK

SEQ ID NO: 71 - protein, 11 aa, Homo sapiens
  MISC_FEATURE (1)..(11): CYP2C19 (Residues 400-410)
EFPNPEMFDPR

SEQ ID NO: 72 - protein, 11 aa, Homo sapiens
  MISC_FEATURE (1)..(11): CYP2C19 (Residues 422-432)
SNYFMPFSAGK

SEQ ID NO: 73 - protein, 10 aa, Homo sapiens
  MISC_FEATURE (1)..(10): CYP2D6 (Residues 205-214)
LLDLAQEGLK

SEQ ID NO: 74 - protein, 14 aa, Homo sapiens
  MISC_FEATURE (1)..(14): CYP2D6 (Residues 246-259)
AFLTQLDELLTEHR

SEQ ID NO: 75 - protein, 10 aa, Homo sapiens
  MISC_FEATURE (1)..(10): CYP2D6 (Residues 260-269)
MTWDPAQPPR

SEQ ID NO: 76 - protein, 12 aa, Homo sapiens
  MISC_FEATURE (1)..(12): CYP2D6 (Residues 270-281)
DLTEAFLAEMEK

SEQ ID NO: 77 - protein, 13 aa, Homo sapiens
  MISC_FEATURE (1)..(13): CYP2D6 (Residues 284-296)
GNPESSFNDENLR

SEQ ID NO: 78 - protein, 13 aa, Homo sapiens
  MISC_FEATURE (1)..(13): CYP2D6 (Residues 331-343)
VQQEIDDVIGQVR

SEQ ID NO: 79 - protein, 15 aa, Homo sapiens
  MISC_FEATURE (1)..(15): CYP2D6 (Residues 366-380)
FGDIVPLGMTHMTSR

SEQ ID NO: 80 - protein, 13 aa, Homo sapiens
  MISC_FEATURE (1)..(13): CYP2D6 (Residues 392-404)
GTTLITNLSSVLK

SEQ ID NO: 81 - protein, 10 aa, Homo sapiens
  MISC_FEATURE (1)..(10): CYP2D6 (Residues 405-414)
DEAVWEKPFR

SEQ ID NO: 82 - protein, 13 aa, Homo sapiens
  MISC_FEATURE (1)..(13): CYP2E1 (Residues 64-76)
FGPVFTLYVGSQR

SEQ ID NO: 83 - protein, 10 aa, Homo sapiens
  MISC_FEATURE (1)..(10): CYP2E1 (Residues 101-110)
GDLPAFHAHR

SEQ ID NO: 84 - protein, 11 aa, Homo sapiens
  MISC_FEATURE (1)..(11): CYP2E1 (Residues 113-123)
GIIFNNGPTWK

SEQ ID NO: 85 - protein, 10 aa, Homo sapiens
  MISC_FEATURE (1)..(10): CYP2E1 (Residues 150-159)
EAHFLLEALR

SEQ ID NO: 86 - protein, 8 aa, Homo sapiens
  MISC_FEATURE (1)..(8): CYP2E1 (Residues 188-195)
HFDYNDEK

SEQ ID NO: 87 - protein, 15 aa, Homo sapiens
  MISC_FEATURE (1)..(15): CYP2E1 (Residues 345-359)
QEMPYMDAVVHEIQR

SEQ ID NO: 88 - protein, 15 aa, Homo sapiens
  MISC_FEATURE (1)..(15): CYP2E1 (Residues 360-374)
FITLVPSNLPHEATR

SEQ ID NO: 89 - protein, 12 aa, Homo sapiens
  MISC_FEATURE (1)..(12): CYP2E1 (Residues 409-420)
FKPEHFLNENGK

SEQ ID NO: 90 - protein, 12 aa, Homo sapiens
  MISC_FEATURE (1)..(12): CYP2E1 (Residues 423-434)
YSDYFKPFSTGK

SEQ ID NO: 91 - protein, 13 aa, Homo sapiens
  MISC_FEATURE (1)..(13): CYP2F1 (Residues 61-73)
EYGSMYTVHLGPR

SEQ ID NO: 92 - protein, 11 aa, Homo sapiens
  MISC_FEATURE (1)..(11): CYP2F1 (Residues 75-85)
VVVLSGYQAVK

SEQ ID NO: 93 - protein, 13 aa, Homo sapiens
  MISC_FEATURE (1)..(13): CYP2F1 (Residues 86-98)
EALVDQGEEFSGR

SEQ ID NO: 94 - protein, 11 aa, Homo sapiens
  MISC_FEATURE (1)..(11): CYP2F1 (Residues 110-120)
GNGIAFSSGDR

SEQ ID NO: 95 - protein, 8 aa, Homo sapiens
  MISC_FEATURE (1)..(8): CYP2F1 (Residues 126-133)
QFSIQILR

SEQ ID NO: 96 - protein, 13 aa, Homo sapiens
  MISC_FEATURE (1)..(13): CYP2F1 (Residues 146-158)
ILEEGSFLLADVR

SEQ ID NO: 97 - protein, 14 aa, Homo sapiens
  MISC_FEATURE (1)..(14): CYP2F1 (Residues 160-173)
TEGEPFDPTFVLSR

SEQ ID NO: 98 - protein, 14 aa, Homo sapiens
  MISC_FEATURE (1)..(14): CYP2F1 (Residues 224-237)
FPSLLDWVPGPHQR

SEQ ID NO: 99 - protein, 16 aa, Homo sapiens
  MISC_FEATURE (1)..(16): CYP2F1 (Residues 247-262)
DLIAHSVHDHQASSPR

SEQ ID NO: 100 - protein, 15 aa, Homo sapiens
  MISC_FEATURE (1)..(15): CYP2F1 (Residues 302-316)
TVSTTLHHAFLALMK

SEQ ID NO: 101 - protein, 15 aa, Homo sapiens
  MISC_FEATURE (1)..(15): CYP2F1 (Residues 324-334)
TVSTTLHHAFLALMK

SEQ ID NO: 102 - protein, 15 aa, Homo sapiens
  MISC_FEATURE (1)..(15): CYP2F1 (Residues 344-358)
AAMPYTDAVIHEVQR

SEQ ID NO: 103 - protein, 12 aa, Homo sapiens
  MISC_FEATURE (1)..(12): CYP2F1 (Residues 359-370)
FADIIPMNLPHR

SEQ ID NO: 104 - protein, 11 aa, Homo sapiens
  MISC_FEATURE (1)..(11): CYP2F1 (Residues 423-433)
SPAFMPFSAGR

SEQ ID NO: 105 - protein, 17 aa, Homo sapiens
  MISC_FEATURE (1)..(17): CYP2J2 (Residues 20-36)
TLLLGTVAFLLAADFLK

SEQ ID NO: 106 - protein, 12 aa, Homo sapiens
  MISC_FEATURE (1)..(12): CYP2J2 (Residues 124-135)
NGLIMSSGQAWK

SEQ ID NO: 107 - protein, 13 aa, Homo sapiens
  MISC_FEATURE (1)..(13): CYP2J2 (Residues 159-171)
IQEEAQHLTEAIK

SEQ ID NO: 108 - protein, 12 aa, Homo sapiens
  MISC_FEATURE (1)..(12): CYP2J2 (Residues 172-183)
EENGQPFDPHFK

SEQ ID NO: 109 - protein, 13 aa, Homo sapiens
  MISC_FEATURE (1)..(13): CYP2J2 (Residues 201-213)
FEYQDSWFQQLLK

SEQ ID NO: 110 - protein, 12 aa, Homo sapiens
  MISC_FEATURE (1)..(12): CYP2J2 (Residues 214-225)
LLDEVTYLEASK

SEQ ID NO: 111 - protein, 14 aa, Homo sapiens
  MISC_FEATURE (1)..(14): CYP2J2 (Residues 239-252)
FLPGPHQTLFSNWK

SEQ ID NO: 112 - protein, 9 aa, Homo sapiens
  MISC_FEATURE (1)..(9): CYP2J2 (Residues 256-264)
LFVSHMIDK

SEQ ID NO: 113 - protein, 13 aa, Homo sapiens
  MISC_FEATURE (1)..(13): CYP2J2 (Residues 344-356)
VIGQGQQPSTAAR

SEQ ID NO: 114 - protein, 15 aa, Homo sapiens
  MISC_FEATURE (1)..(15): CYP2J2 (Residues 357-371)
ESMPYTNAVIHEVQR

SEQ ID NO: 115 - protein, 11 aa, Homo sapiens
  MISC_FEATURE (1)..(11): CYP2J2 (Residues 372-382)
MGNIIPLNVPR

SEQ ID NO: 116 - protein, 15 aa, Homo sapiens
  MISC_FEATURE (1)..(15): CYP2J2 (Residues 383-397)
EVTVDTTLAGYHLPK

SEQ ID NO: 117 - protein, 13 aa, Homo sapiens
  MISC_FEATURE (1)..(13): CYP2J2 (Residues 398-410)
GTMILTNLTALHR

SEQ ID NO: 118 - protein, 10 aa, Homo sapiens
  MISC_FEATURE (1)..(10): CYP2J2 (Residues 436-445)
EAFMPFSIGK

SEQ ID NO: 119 - protein, 13 aa, Homo sapiens
  MISC_FEATURE (1)..(13): CYP2J2 (Residues 456-468)
TELFIFFTSLMQK

SEQ ID NO: 120 - protein, 10 aa, Homo sapiens
  MISC_FEATURE (1)..(10): CYP2J2 (Residues 469-478)
FTFRPPNNEK

SEQ ID NO: 121 - protein, 11 aa, Homo sapiens
  MISC_FEATURE (1)..(11): CYP2J2 (Residues 485-495)
MGITISPVSHR

SEQ ID NO: 122 - protein, 22 aa, Homo sapiens
  MISC_FEATURE (1)..(22): CYP2R1 (Residues 7-28)
AEEGAAALGGALFLLLFALGVR

SEQ ID NO: 123 - protein, 10 aa, Homo sapiens
  MISC_FEATURE (1)..(10): CYP2R1 (Residues 164-173)
FFNDAIETYK

SEQ ID NO: 124 - protein, 19 aa, Homo sapiens
  MISC_FEATURE (1)..(19): CYP2R1 (Residues 181-199)
QLITNAVSNITNLIIFGER

SEQ ID NO: 125 - protein, 11 aa, Homo sapiens
  MISC_FEATURE (1)..(11): CYP2R1 (Residues 249-259)
NAAVVYDFLSR

SEQ ID NO: 126 - protein, 9 aa, Homo sapiens
  MISC_FEATURE (1)..(9): CYP2R1 (Residues 289-297)
NDPSSTFSK

SEQ ID NO: 127 - protein, 13 aa, Homo sapiens
  MISC_FEATURE (1)..(13): CYP2R1 (Residues 358-370)
MPYTEAVLHEVLR

SEQ ID NO: 128 - protein, 16 aa, Homo sapiens
  MISC_FEATURE (1)..(16): CYP2R1 (Residues 397-412)
GTTVITNLYSVHFDEK

SEQ ID NO: 129 - protein, 9 aa, Homo sapiens
  MISC_FEATURE (1)..(9): CYP2R1 (Residues 416-424)
DPEVFHPER

SEQ ID NO: 130 - protein, 10 aa, Homo sapiens
  MISC_FEATURE (1)..(10): CYP2R1 (Residues 425-434)
FLDSSGYFAK

SEQ ID NO: 131 - protein, 10 aa, Homo sapiens
  MISC_FEATURE (1)..(10): CYP2R1 (Residues 436-445)
EALVPFSLGR

SEQ ID NO: 132 - protein, 9 aa, Homo sapiens
  MISC_FEATURE (1)..(9): CYP2R1 (Residues 447-455)
HCLGEHLAR

SEQ ID NO: 133 - protein, 13 aa, Homo sapiens
  MISC_FEATURE (1)..(13): CYP2R1 (Residues 456-468)
MEMFLFFTALLQR

SEQ ID NO: 134 - protein, 16 aa, Homo sapiens
  MISC_FEATURE (1)..(16): CYP2R1 (Residues 469-484)
FHLHFPHELVPDLKPR

SEQ ID NO: 135 - protein, 16 aa, Homo sapiens
  MISC_FEATURE (1)..(16): CPY2R1 (Residues 485-500)
LGMTLQPQPYLICAER

SEQ ID NO: 136 - protein, 13 aa, Homo sapiens
  MISC_FEATURE (1)..(13): CYP2S1 (Residues 89-101)
EALGGQAEEFSGR

SEQ ID NO: 137 - protein, 11 aa, Homo sapiens
  MISC_FEATURE (1)..(11): CYP2S1 (Residues 144-154)
EGEELIQAEAR

SEQ ID NO: 138 - protein, 15 aa, Homo sapiens
  MISC_FEATURE (1)..(15): CYP2S1 (Residues 236-250)
QLLHHVSTLAAFTVR

SEQ ID NO: 139 - protein, 16 aa, Homo sapiens
  MISC_FEATURE (1)..(16): CYP2S1 (Residues 251-266)
QVQQHQGNLDASGPAR

SEQ ID NO: 140 - protein, 9 aa, Homo sapiens
  MISC_FEATURE (1)..(9): CYP2S1 (Residues 267-275)
DLVDAFLLK

SEQ ID NO: 141 - protein, 15 aa, Homo sapiens
  MISC_FEATURE (1)..(15): CYP2S1 (Residues 276-290)
MAQEEQNPGTEFTNK

SEQ ID NO: 142 - protein, 13 aa, Homo sapiens
  MISC_FEATURE (1)..(13): CYP2S1 (Residues 335-347)
ELGAGQAPSLGDR

SEQ ID NO: 143 - protein, 13 aa, Homo sapiens
  MISC_FEATURE (1)..(13): CYP2S1 (Residues 350-362)
LPYTDAVLHEAQR

SEQ ID NO: 144 - protein, 11 aa, Homo sapiens
  MISC_FEATURE (1)..(11): CYP2S1 (Residues 363-373)
LLALVPMGIPR

SEQ ID NO: 145 - protein, 9 aa, Homo sapiens
  MISC_FEATURE (1)..(9): CYP2S1 (Residues 408-416)
HPEEFNPDR

SEQ ID NO: 146 - protein, 11 aa, Homo sapiens
  MISC_FEATURE (1)..(11): CYP2S1 (Residues 427-437)
HEAFLPFSLGK

SEQ ID NO: 147 - protein, 19 aa, Homo sapiens
  MISC_FEATURE (1)..(19): CYP2U1 (Residues 1-19)
MSSPGPSQPPAEDPPWPAR

SEQ ID NO: 148 - protein, 20 aa, Homo sapiens
  MISC_FEATURE (1)..(20): CYP2U1 (Residues 93-112)
AAGIDPSVIGPQVLLAHLAR

SEQ ID NO: 149 - protein, 12 aa, Homo sapiens
  MISC_FEATURE (1)..(12): CYP2U1 (Residues 164-175)
GVVFAHYGPVWR

SEQ ID NO: 150 - protein, 9 aa, Homo sapiens
  MISC_FEATURE (1)..(9): CYP2U1 (Residues 241-249)
FDYTNSEFK

SEQ ID NO: 151 - protein, 8 aa, Homo sapiens
  MISC_FEATURE (1)..(8): CYP2U1 (Residues 304-311)
DHQESLDR

SEQ ID NO: 152 - protein, 15 aa, Homo sapiens
  MISC_FEATURE (1)..(15): CYP2U1 (Residuces 398-412)
AQMPYTEATIMEVQR

SEQ ID NO: 153 - protein, 13 aa, Homo sapiens
  MISC_FEATURE (1)..(13): CYP2U1 (Residues 439-451)
GTLILPNLWSVHR

SEQ ID NO: 154 - protein, 15 aa, Homo sapiens
  MISC_FEATURE (1)..(15): CYP2U1 (Residues 452-466)
DPAIWEKPEDFYPNR

SEQ ID NO: 155 - protein, 10 aa, Homo sapiens
  MISC_FEATURE (1)..(10): CYP2U1 (Residues 467-476)
FLDDQGQLIK

SEQ ID NO: 156 - protein, 10 aa, Homo sapiens
  MISC_FEATURE (1)..(10): CYP2U1 (Residues 478-487)
ETFIPFGIGK

SEQ ID NO: 157 - protein, 16 aa, Homo sapiens
  MISC_FEATURE (1)..(16): CYP2U1 (Residues 528-543)
FGLTLAPHPFNITISR

SEQ ID NO: 158 - protein, 16 aa, Homo sapiens
  MISC_FEATURE (1)..(16): CYP2W1 (Residues 7-17)
FGLTLAPHPFNITISR

SEQ ID NO: 159 - protein, 11 aa, Homo sapiens
  MISC_FEATURE (1)..(11): CYP2W1 (Residues 20-30)
TVVLTGFEAVK

SEQ ID NO: 160 - protein, 11 aa, Homo sapiens
  MISC_FEATURE (1)..(11): CYP2W1 (Residues 55-65)
GGGIFFSSGAR

SEQ ID NO: 161 - protein, 10 aa, Homo sapiens
  MISC_FEATURE (1)..(10): CYP2W1 (Residues 97-106)
CLSGQLDGYR

SEQ ID NO: 162 - protein, 19 aa, Homo sapiens
  MISC_FEATURE (1)..(19): CYP2W1 (Residues 282-300)
LEDQQALPYTSAVLHEVQR

SEQ ID NO: 163 - protein, 10 aa, Homo sapiens
  MISC_FEATURE (1)..(10): CYP2W1 (Residues 301-310)
FITLLPHVPR

SEQ ID NO: 164 - protein, 15 aa, Homo sapiens
  MISC_FEATURE (1)..(15): CYP2W1 (Residues 311-325)
CTAADTQLGGFLLPK

SEQ ID NO: 165 - protein, 10 aa, Homo sapiens
  MISC_FEATURE (1)..(10): CYP2W1 (Residues 365-374)
EAFLPFSAGR

SEQ ID NO: 166 - protein, 18 aa, Homo sapiens
  MISC_FEATURE (1)..(18): CYP2W1 (Residues 400-417)
LLPPPGVSPASLDTTPAR

SEQ ID NO: 167 - protein, 10 aa, Homo sapiens
  MISC_FEATURE (1)..(10): CYP3A3 (Residues 105-114)
EPFGPVGFMK

SEQ ID NO: 168 - protein, 12 aa, Homo sapiens
  MISC_FEATURE (1)..(12): CYP3A3 (Residues 268-279)
VDFLQLMIDSHK

SEQ ID NO: 169 - protein, 20 aa, Homo sapiens
  MISC_FEATURE (1)..(20): CYP3A4 (Residues 35-54)
LGIPGPTPLPFLGNILSYHK

SEQ ID NO: 170 - protein, 21 aa, Homo sapiens
  MISC_FEATURE (1)..(21): CYP3A4 (Residues 70-90)
VWGFYDGQQPVLAITDPDMIK

SEQ ID NO: 171 - protein, 12 aa, Homo sapiens
  MISC_FEATURE (1)..(12): CYP3A4 (Residues 115-126)
SAISIAEDEEWK

SEQ ID NO: 172 - protein, 7 aa, Homo sapiens
  MISC_FEATURE (1)..(7): CYP3A4 (Residues 243-249)
EVTNFLR

SEQ ID NO: 173 - protein, 14 aa, Homo sapiens
  MISC_FEATURE (1)..(14): CYP3A4 (Residues 268-281)
VDFLQLMIDSQNSK

SEQ ID NO: 174 - protein, 13 aa, Homo sapiens
  MISC_FEATURE (1)..(13): CYP3A4 (Residues 390-402)
GWVVMIPSYALHR

SEQ ID NO: 175 - protein, 16 aa, Homo sapiens
  MISC_FEATURE (1)..(16): CYP3A4 (Residues 476-491)
LSLGGLLQPEKPVVLK

SEQ ID NO: 176 - protein, 12 aa, Homo sapiens
  MISC_FEATURE (1)..(12): CYP3A5 (Residues 116-127)
SAISLAEDEEWK

SEQ ID NO: 177 - protein, 15 aa, Homo sapiens
  MISC_FEATURE (1)..(15): CYP3A5 (Residues 144-158)
EMFPIIAQYGDVLVR

SEQ ID NO: 178 - protein, 14 aa, Homo sapiens
  MISC_FEATURE (1)..(14): CYP3A5 (Residues 269-282)
LDFLQLMIDSQNSK

SEQ ID NO: 179 - protein, 11 aa, Homo sapiens
  MISC_FEATURE (1)..(11): CYP3A5 (Residues 380-390)
DVEINGVFIPK

SEQ ID NO: 180 - protein, 16 aa, Homo sapiens
  MISC_FEATURE (1)..(16): CYP3A5 (Residues 391-406)
GSMVVIPTYALHHDPK

SEQ ID NO: 181 - protein, 12 aa, Homo sapiens
  MISC_FEATURE (1)..(12): CYP3A5 (Residues 407-418)
YWTEPEEFRPER

SEQ ID NO: 182 - protein, 16 aa, Homo sapiens
  MISC_FEATURE (1)..(16): CYP3A5 (Residues 476-491)
LDTQGLLQPEKPIVLK

SEQ ID NO: 183 - protein, 12 aa, Homo sapiens
  MISC_FEATURE (1)..(12): CYP3A7 (Residues 116-127)
NAISIAEDEEWK

SEQ ID NO: 184 - protein, 12 aa, Homo sapiens
  MISC_FEATURE (1)..(12): CYP3A7 (Residues 213-224)
FNPLDPFVLSIK

SEQ ID NO: 185 - protein, 9 aa, Homo sapiens
  MISC_FEATURE (1)..(9): CYP3A7 (Residues 334-342)
EIDTVLPNK

SEQ ID NO: 186 - protein, 16 aa, Homo sapiens
  MISC_FEATURE (1)..(16): CYP3A7 (Residues 391-406)
GVVVMIPSYVLHHDPK

SEQ ID NO: 187 - protein, 14 aa, Homo sapiens
  MISC_FEATURE (1)..(14): CYP3A7 (Residues 479-492)
FGGLLLTEKPIVLK

SEQ ID NO: 188 - protein, 7 aa, Homo sapiens
  MISC_FEATURE (1)..(7): CYP3A43 (Residues 56-62)
GLWNFDR

SEQ ID NO: 189 - protein, 12 aa, Homo sapiens
  MISC_FEATURE (1)..(12): CYP3A43 (Residues 116-127)
SALSFAEDEEWK

SEQ ID NO: 190 - protein, 11 aa, Homo sapiens
  MISC_FEATURE (1)..(11): CYP3A43 (Residues 131-141)
TLLSPAFTSVK

SEQ ID NO: 191 - protein, 14 aa, Homo sapiens
  MISC_FEATURE (1)..(14): CYP3A43 (Residues 269-282)
VDFFQQMIDSQNSK

SEQ ID NO: 192 - protein, 16 aa, Homo sapiens
  MISC_FEATURE (1)..(16): CYP3A43 (Residues 391-409)
GLAVMVPIYALHHDPK

SEQ ID NO: 193 - protein, 9 aa, Homo sapiens
  MISC_FEATURE (1)..(9): CYP3A43 (Residues 432-440)
YIPFGAGPR

SEQ ID NO: 194 - protein, 16 aa, Homo sapiens
  MISC_FEATURE (1)..(16): CYP3A43 (Residues 477-492)
LDNLPILQPEKPIVLK

SEQ ID NO: 195 - protein, 10 aa, Homo sapiens
  MISC_FEATURE (1)..(10): CYP4A11 (Residues 1-10)
MSVSVLSPSR

SEQ ID NO: 196 - protein, 8 aa, Homo sapiens
  MISC_FEATURE (1)..(8): CYP4A11 (Residues 34-41)
AVQLYLHR

SEQ ID NO: 197 - protein, 10 aa, Homo sapiens
  MISC_FEATURE (1)..(10): CYP4A11 (Residues 97-106)
VQLYDPDYMK

SEQ ID NO: 198 - protein, 17 aa, Homo sapiens
  MISC_FEATURE (1)..(17): CYP4A11 (Residues 234-250)
NAFHQNDTIYSLTSAGR

SEQ ID NO: 199 - protein, 12 aa, Homo sapiens
  MISC_FEATURE (1)..(12): CYP4A11 (Residues 288-299)
HLDFLDILLLAK

SEQ ID NO: 200 - protein, 10 aa, Homo sapiens
  MISC_FEATURE (1)..(10): CYP4A11 (Residues 300-309)
MENGSILSDK

SEQ ID NO: 201 - protein, 12 aa, Homo sapiens
  MISC_FEATURE (1)..(12): CYP4A11 (Residues 392-403)
ELSTPVTFPDGR

SEQ ID NO: 202 - protein, 16 aa, Homo sapiens
  MISC_FEATURE (1)..(16): CYP4A11 (Residues 408-423)
GIMVLLSIYGLHHNPK

SEQ ID NO: 203 - protein, 12 aa, Homo sapiens
  MISC_FEATURE (1)..(12): CYP4A11 (Residues 424-435)
VWPNPEVFDPFR

SEQ ID NO: 204 - protein, 9 aa, Homo sapiens
  MISC_FEATURE (1)..(9): CYP4A11 (Residues 478-486)
FELLPDPTR

SEQ ID NO: 205 - protein, 14 aa, Homo sapiens
  MISC_FEATURE (1)..(14): CYP4B1 (Residues 111-124)
APDVYDFFLQWIGR

SEQ ID NO: 206 - protein, 12 aa, Homo sapiens
  MISC_FEATURE (1)..(12): CYP4B1 (Residues 282-293)
HLDFLDILLGAR

SEQ ID NO: 207 - protein, 16 aa, Homo sapiens
  MISC_FEATURE (1)..(16): CYP4B1 (Residues 347-362)
EILGDQDFFQWDDLGK

SEQ ID NO: 208 - protein, 10 aa, Homo sapiens
  MISC_FEATURE (1)..(10): CYP4B1 (Residues 376-385)
LYPPVPQVYR

SEQ ID NO: 209 - protein, 12 aa, Homo sapiens
  MISC_FEATURE: CYP4B1 (Residues 386-397)
QLSKPVTFVDGR

SEQ ID NO: 210 - protein, 17 aa, Homo sapiens
  MISC_FEATURE (1)..(17): CYP4B1 (Residues 398-414)
SLPAGSLISMHIYALHR

SEQ ID NO: 211 - protein, 15 aa, Homo sapiens
  MISC_FEATURE (1)..(15): CYP4B1 (Residues 415-429)
NSAVWPDPEVFDSLR

SEQ ID NO: 212 - protein, 13 aa, Homo sapiens
  MISC_FEATURE (1)..(13): CYP4B1 (Residues 439-451)
HPFAFMPFSAGPR

SEQ ID NO: 213 - protein, 9 aa, Homo sapiens
  MISC_FEATURE (1)..(9): CYP4B1 (Residues 474-482)
FEFSLDPSR

SEQ ID NO: 214 - protein, 18 aa, Homo sapiens
  MISC_FEATURE (1)..(18): CYP4F2 (Residues 58-75)
NWFWGHQGMVNPTEEGMR

SEQ ID NO: 215 - protein, 14 aa, Homo sapiens
  MISC_FEATURE (1)..(14): CYP4F2 (Residues 76-89)
VLTQLVATYPQGFK

SEQ ID NO: 216 - protein, 19 aa, Homo sapiens
  MISC_FEATURE (1)..(19): CYP4F2 (Residues 90-108)
VWMGPISPLLSLCHPDIIR

SEQ ID NO: 217 - protein, 12 aa, Homo sapiens
  MISC_FEATURE (1)..(12): CYP4F2 (Residues 109-120)
SVINASAAIAPK

SEQ ID NO: 218 - protein, 18 aa, Homo sapiens
  MISC_FEATURE (1)..(18): CYP4F2 (Residues 369-386)
EIEWDDLAHLPFLTMCMK

SEQ ID NO: 219 - protein, 12 aa, Homo sapiens
  MISC_FEATURE (1)..(12): CYP4F2 (Residues 401-412)
HVTQDIVLPDGR

SEQ ID NO: 220 - protein, 13 aa, Homo sapiens
  MISC_FEATURE (1)..(13): CYP4F2 (Residues 467-479)
NCIGQTFAMAEMK

SEQ ID NO: 221 - protein, 14 aa, Homo sapiens
  MISC_FEATURE (1)..(14): CYP4F3 (Residues 34-47)
ILAWTYTFYDNCCR

SEQ ID NO: 222 - protein, 20 aa, Homo sapiens
  MISC_FEATURE (1)..(20): CYP4F3 (Residues 101-120)
IFHPTYIKPVLFAPAAIVPK

SEQ ID NO: 223 - protein, 11 aa, Homo sapiens
  MISC_FEATURE (1)..(11): CYP4F3 (Residues 178-188)
WQLLASEGSAR

SEQ ID NO: 224 - protein, 10 aa, Homo sapiens
  MISC_FEATURE (1)..(10): CYP4F3 (Residues 391-400)
LHPPVPAVSR

SEQ ID NO: 225 - protein, 9 aa, Homo sapiens
  MISC_FEATURE (1)..(9): CYP4F3 (Residues 480-488)
VVLGLTLLR

SEQ ID NO: 226 - protein, 13 aa, Homo sapiens
  MISC_FEATURE (1)..(13): CYP4F8 (Residues 34-46)
ILAWTYAFYHNGR

SEQ ID NO: 227 - protein, 19 aa, Homo sapiens
  MISC_FEATURE (1)..(19): CYP4F8 (Residues 57-75)
QNWFLGHLGLVTPTEEGLR

SEQ ID NO: 228 - protein, 15 aa, Homo sapiens
  MISC_FEATURE (1)..(15): CYP4F8 (Residues 76-90)
VLTQLVATYPQGFVR

SEQ ID NO: 229 - protein, 18 aa, Homo sapiens
  MISC_FEATURE (1)..(18): CYP4F8 (Residues 91-108)
WLGPITPIINLCHPDIVR

SEQ ID NO: 230 - protein, 12 aa, Homo sapiens
  MISC_FEATURE (1)..(12): CYP4F8 (Residues 109-120)
SVINTSDAITDK

SEQ ID NO: 231 - protein, 17 aa, Homo sapiens
  MISC_FEATURE (1)..(17): CYP4F8 (Residues 127-143)
TLKPWLGDGLLLSVGDK

SEQ ID NO: 232 - protein, 16 aa, Homo sapiens
  MISC_FEATURE (1)..(16): CYP4F8 (Residues 150-165)
LLTPAFHFNILKPYIK

SEQ ID NO: 233 - protein, 11 aa, Homo sapiens
  MISC_FEATURE (1)..(11): CYP4F8 (Residues 244-254)
DFLYFLTPCGR

SEQ ID NO: 234 - protein, 14 aa, Homo sapiens
  MISC_FEATURE (1)..(14): CYP4F8 (Residues 277-290)
TLTSQGVDDFLQAK

SEQ ID NO: 235 - protein, 14 aa, Homo sapiens
  MISC_FEATURE (1)..(14): CYP4F8 (Residues 295-308)
TLDFIDVLLLSEDK

SEQ ID NO: 236 - protein, 18 aa, Homo sapiens
  MISC_FEATURE (1)..(18): CYP4F8 (Residues 369-386)
EIEWDDLAQLPFLTMCLK

SEQ ID NO: 237 - protein, 10 aa, Homo sapiens
  MISC_FEATURE (1)..(10): CYP4F8 (Residues 391-400)
LHPPIPTFAR

SEQ ID NO: 238 - protein, 12 aa, Homo sapiens
  MISC_FEATURE (1)..(12): CYP4F8 (Residues 401-412)
GCTQDVVLPDSR

SEQ ID NO: 239 - protein, 13 aa, Homo sapiens
  MISC_FEATURE (1)..(13): CYP4F8 (Residues 454-466)
SPMAFIPFSAGPR

SEQ ID NO: 240 - protein, 8 aa, Homo sapiens
  MISC_FEATURE (1)..(8): CYP4F8 (Residues 508-515)
AEDGLWLR

SEQ ID NO: 241 - protein, 13 aa, Homo sapiens
  MISC_FEATURE (1)..(13): CYP4F11 (Residues 34-46)
VLAWTYTFYDNCR

SEQ ID NO: 242 - protein, 14 aa, Homo sapiens
  MISC_FEATURE (1)..(14): CYP4F11 (Residues 76-89)
TLTQLVTTYPQGFK

SEQ ID NO: 243 - protein, 8 aa, Homo sapiens
  MISC_FEATURE (1)..(8): CYP4F11 (Residues 170-177)
SVNIMHDK

SEQ ID NO: 244 - protein, 16 aa, Homo sapiens
  MISC_FEATURE (1)..(16): CYP4F11 (Residues 259-274)
ACHLVHDFTDAVIQER

SEQ ID NO: 245 - protein, 12 aa, Homo sapiens
  MISC_FEATURE (1)..(12): CYP4F11 (Residues 277-288)
TLPTQGIDDFLK

SEQ ID NO: 246 - protein, 9 aa, Homo sapiens
  MISC_FEATURE (1)..(9): CYP4F11 (Residues 347-355)
HPEYQEQCR

SEQ ID NO: 247 - protein, 12 aa, Homo sapiens
  MISC_FEATURE (1)..(12): CYP4F11 (Residues 401-412)
CCTQDFVLPDGR

SEQ ID NO: 248 - protein, 11 aa, Homo sapiens
  MISC_FEATURE (1)..(11): CYP4F11 (Residues 480-490)
VVLALTLLHFR

SEQ ID NO: 249 - protein, 9 aa, Homo sapiens
  MISC_FEATURE (1)..(9): CYP4F11 (Residues 491-499)
ILPTHTEPR

SEQ ID NO: 250 - protein, 13 aa, Homo sapiens
  MISC_FEATURE (1)..(13): CYP4F12 (Residues 34-46)
ILAWTYAFYNNCR

SEQ ID NO: 251 - protein, 18 aa, Homo sapiens
  MISC_FEATURE (1)..(18): CYP4F12 (Residues 58-75)
NWFWGHLGLITPTEEGLK

SEQ ID NO: 252 - protein, 12 aa, Homo sapiens
  MISC_FEATURE (1)..(12): CYP4F12 (Residues 109-120)
SITNASAAIAPK

SEQ ID NO: 253 - protein, 17 aa, Homo sapiens
  MISC_FEATURE (1)..(17): CYP4F12 (Residues 127-143)
FLKPWLGEGILLSGGDK

SEQ ID NO: 254 - protein, 8 aa, Homo sapiens
  MISC_FEATURE (1)..(8): CYP4F12 (Residues 162-169)
SYITIFNK

SEQ ID NO: 255 - protein, 11 aa, Homo sapiens
  MISC_FEATURE (1)..(11): CYP4F12 (Residues 178-188)
WQHLASEGSSR

SEQ ID NO: 256 - protein, 11 aa, Homo sapiens
  MISC_FEATURE (1)..(11): CYP4F12 (Residues 262-272)
LVHDFTDAVIR

SEQ ID NO: 257 - protein, 12 aa, Homo sapiens
  MISC_FEATURE (1)..(12): CYP4F12 (Residues 277-288)
TLPTQGIDDFFK

SEQ ID NO: 258 - protein, 10 aa, Homo sapiens
  MISC_FEATURE (1)..(10): CYP4F12 (Residues 391-400)
LHPPAPFISR

SEQ ID NO: 259 - protein, 11 aa, Homo sapiens
  MISC_FEATURE (1)..(11): CYP4F12 (Residues 480-490)
VVLALMLLHFR

SEQ ID NO: 260 - protein, 9 aa, Homo sapiens
  MISC_FEATURE (1)..(9): CYP4F12 (Residues 491-499)
FLPDHTEPR

SEQ ID NO: 261 - protein, 9 aa, Homo sapiens
  MISC_FEATURE (1)..(9): CYP4F12 (Residues 516-524)
VEPLNVGLQ

SEQ ID NO: 262 - protein, 12 aa, Homo sapiens
  MISC_FEATURE (1)..(12): CYP4V2 (Residues 1-12)
MAGLWLGLVWQK

SEQ ID NO: 263 - protein, 16 aa, Homo sapiens
  MISC_FEATURE (1)..(16): CYP4V2 (Residues 56-71)
AYPLVGHALLMKPDGR

SEQ ID NO: 264 - protein, 14 aa, Homo sapiens
  MISC_FEATURE (1)..(14): CYP4V2 (Residues 72-85)
EFFQQIIEYTEEYR

SEQ ID NO: 265 - protein, 17 aa, Homo sapiens
  MISC_FEATURE (1)..(17): CYP4V2 (Residues 126-142)
FLEPWLGLGLLTSTGNK

SEQ ID NO: 266 - protein, 14 aa, Homo sapiens
  MISC_FEATURE (1)..(14): CYP4V2 (Residues 208-221)
NIGAQSNDDSEYVR

SEQ ID NO: 267 - protein, 14 aa, Homo sapiens
  MISC_FEATURE (1)..(14): CYP4V2 (Residues 236-249)
MPWLWLDLWYLMFK

SEQ ID NO: 268 - protein, 13 aa, Homo sapiens
  MISC_FEATURE (1)..(13): CYP4V2 (Residues 260-272)
ILHTFTNSVIAER

SEQ ID NO: 269 - protein, 11 aa, Homo sapiens
  MISC_FEATURE (1)..(11): CYP4V2 (Residues 273-283)
ANEMNANEDCR

SEQ ID NO: 270 - protein, 16 aa, Homo sapiens
  MISC_FEATURE (1)..(16): CYP4V2 (Residues 298-313)
AFLDLLLSVTDDEGNR

SEQ ID NO: 271 - protein, 11 aa, Homo sapiens
  MISC_FEATURE (1)..(11): CYP4V2 (Residues 355-365)
VDHELDDVFGK

SEQ ID NO: 272 - protein, 11 aa, Homo sapiens
  MISC_FEATURE (1)..(11): CYP4V2 (Residues 366-376)
SDRPATVEDLK

SEQ ID NO: 273 - protein, 10 aa, Homo sapiens
  MISC_FEATURE (1)..(10): CYP4V2 (Residues 391-400)
LFPSVPLFAR

SEQ ID NO: 274 - protein, 12 aa, Homo sapiens
  MISC_FEATURE (1)..(12): CYP4V2 (Residues 401-412)
SVSEDCEVAGYR

SEQ ID NO: 275 - protein, 13 aa, Homo sapiens
  MISC_FEATURE (1)..(13): CYP4V2 (Residues 416-428)
GTEAVIIPYALHR

SEQ ID NO: 276 - protein, 12 aa, Homo sapiens
  MISC_FEATURE (1)..(12): CYP4V2 (Residues 432-443)
YFPNPEEFQPER

SEQ ID NO: 277 - protein, 9 aa, Homo sapiens
  MISC_FEATURE (1)..(9): CYP4V2 (Residues 444-452)
FFPENAQGR

SEQ ID NO: 278 - protein, 13 aa, Homo sapiens
  MISC_FEATURE (1)..(13): CYP4V2 (Residues 453-465)
HPYAYVPFSAGPR

SEQ ID NO: 279 - protein, 9 aa, Homo sapiens
  MISC_FEATURE (1)..(9): CYP4V2 (Residues 487-495)
HFWIESNQK

SEQ ID NO: 280 - protein, 9 aa, Homo sapiens
  MISC_FEATURE (1)..(9): CYP4X1 (Residues 1-9)
MEFSWLETR

SEQ ID NO: 281 - protein, 9 aa, Homo sapiens
  MISC_FEATURE (1)..(9): CYP4X1 (Residues 60-68)
FIQDDNMEK

SEQ ID NO: 282 - protein, 12 aa, Homo sapiens
  MISC_FEATURE (1)..(12): CYP4X1 (Residues 139-150)
LLTPGFHFNILK

SEQ ID NO: 283 - protein, 11 aa, Homo sapiens
  MISC_FEATURE (1)..(11): CYP4X1 (Residues 151-161)
AYIEVMAHSVK

SEQ ID NO: 284 - protein, 15 aa, Homo sapiens
  MISC_FEATURE (1)..(15): CYP4X1 (Residues 200-214)
ETNCQTNSTHDPYAK

SEQ ID NO: 285 - protein, 13 aa, Homo sapiens
  MISC_FEATURE (1)..(13): CYP4X1 (Residues 227-239)
LYSLLYHSDIIFK

SEQ ID NO: 286 - protein, 13 aa, Homo sapiens
  MISC_FEATURE (1)..(13): CYP4X1 (Residues 253-265)
VLNQYTDTIIQER

SEQ ID NO: 287 - protein, 12 aa, Homo sapiens
  MISC_FEATURE (1)..(12): CYP4X1 (Residues 283-294)
YQDFLDIVLSAK

SEQ ID NO: 288 - protein, 10 aa, Homo sapiens
  MISC_FEATURE (1)..(10): CYP4X1 (Residues 377-386)
LIPAVPSISR

SEQ ID NO: 289 - protein, 9 aa, Homo sapiens
  MISC_FEATURE (1)..(9): CYP4X1 (Residues 431-439)
FSQENSDQR

SEQ ID NO: 290 - protein, 13 aa, Homo sapiens
  MISC_FEATURE (1)..(13): CYP4X1 (Residues 440-452)
HPYAYLPFSAGSR

SEQ ID NO: 291 - protein, 13 aa, Homo sapiens
  MISC_FEATURE (1)..(13): CYP4X1 (Residues 453-465)
NCIGQEFAMIELK

SEQ ID NO: 292 - protein, 11 aa, Homo sapiens
  MISC_FEATURE (1)..(11): CYP4X1 (Residues 466-476)
VTIALILLHFR

SEQ ID NO: 293 - protein, 17 aa, Homo sapiens
  MISC_FEATURE (1)..(17): CYP4Z1 (Residues 42-58)
ALHLFPAPPAHWFYGHK

SEQ ID NO: 294 - protein, 8 aa, Homo sapiens
  MISC_FEATURE (1)..(8): CYP4Z1 (Residues 116-123)
ILESWVGR

SEQ ID NO: 295 - protein, 13 aa, Homo sapiens
  MISC_FEATURE (1)..(13): CYP4Z1 (Residues 138-150)
QIVKPGFNISILK

SEQ ID NO: 296 - protein, 13 aa, Homo sapiens
  MISC_FEATURE (1)..(13): CYP4Z1 (Residues 151-161)
QIVKPGFNISILK

SEQ ID NO: 297 - protein, 10 aa, Homo sapiens
  MISC_FEATURE (1)..(10): CYP4Z1 (Residues 167-176)
WEEHIAQNSR

SEQ ID NO: 298 - protein, 17 aa, Homo sapiens
  MISC_FEATURE (1)..(17): CYP4Z1 (Residues 177-193)
LELFQHVSLMTLDSIMK

SEQ ID NO: 299 - protein, 13 aa, Homo sapiens
  MISC_FEATURE (1)..(13): CYP4Z1 (Residues 226-238)
MNNFLHHNDLVFK

SEQ ID NO: 300 - protein, 10 aa, Homo sapiens
  MISC_FEATURE (1)..(10): CYP4Z1 (Residues 239-248)
FSSQGQIFSK

SEQ ID NO: 301 - protein, 11 aa, Homo sapiens
  MISC_FEATURE (1)..(11): CYP4Z1 (Residues 249-259)
FNQELHQFTEK

SEQ ID NO: 302 - protein, 11 aa, Homo sapiens
  MISC_FEATURE (1)..(11): CYP4Z1 (Residues 282-292)
WDFLDILLSAK

SEQ ID NO: 303 - protein, 12 aa, Homo sapiens
  MISC_FEATURE (1)..(12): CYP4Z1 (Residues 298-309)
DFSEADLQAEVK

SEQ ID NO: 304 - protein, 10 aa, Homo sapiens
  MISC_FEATURE (1)..(10): CYP4Z1 (Residues 375-384)
LYAPVVNISR

SEQ ID NO: 305 - protein, 12 aa, Homo sapiens
  MISC_FEATURE (1)..(12): CYP4Z1 (Residues 385-396)
LLDKPITFPDGR

SEQ ID NO: 306 - protein, 14 aa, Homo sapiens
  MISC_FEATURE (1)..(14): CYP4Z1 (Residues 437-450)
IHPYAFIPFSAGLR

SEQ ID NO: 307 - protein, 13 aa, Homo sapiens
  MISC_FEATURE (1)..(13): CYP4Z1 (Residues 451-463)
NCIGQHFAIIECK

SEQ ID NO: 308 - protein, 9 aa, Homo sapiens
  MISC_FEATURE (1)..(9): CYP4Z1 (Residues 464-472)
VAVALTLLR

SEQ ID NO: 309 - protein, 13 aa, Homo sapiens
  MISC_FEATURE (1)..(13): CYP4Z1 (Residues 475-487)
LAPDHSRPPQPVR

SEQ ID NO: 310 - protein, 9 aa, Homo sapiens
  MISC_FEATURE (1)..(9): CYP5A1 (Residues 30-38)
WYSTSAFSR

SEQ ID NO: 311 - protein, 15 aa, Homo sapiens
  MISC_FEATURE (1)..(15): CYP5A1 (Residues 46-60)
HPKPSPFIGNLTFFR

SEQ ID NO: 312 - protein, 11 aa, Homo sapiens
  MISC_FEATURE (1)..(11): CYP5A1 (Residues 61-71)
QGFWESQMELR

SEQ ID NO: 313 - protein, 12 aa, Homo sapiens
  MISC_FEATURE (1)..(12): CYP5A1 (Residues 73-84)
LYGPLCGYYLGR

SEQ ID NO: 314 - protein, 12 aa, Homo sapiens
  MISC_FEATURE (1)..(12): CYP5A1 (Residues 86-97)
MFIVISEPDMIK

SEQ ID NO: 315 - protein, 13 aa, Homo sapiens
  MISC_FEATURE (1)..(13): CYP5A1 (Residues 98-110)
QVLVENFSNFTNR

SEQ ID NO: 316 - protein, 10 aa, Homo sapiens
  MISC_FEATURE (1)..(10): CYP5A1 (Residues 119-128)
SVADSVLFLR

SEQ ID NO: 317 - protein, 11 aa, Homo sapiens
  MISC_FEATURE (1)..(11): CYP5A1 (Residues 137-147)
GALMSAFSPEK

SEQ ID NO: 318 - protein, 12 aa, Homo sapiens
  MISC_FEATURE (1)..(12): CYP5A1 (Residues 169-180)
YAESGDAFDIQR

SEQ ID NO: 319 - protein, 9 aa, Homo sapiens
  MISC_FEATURE (1)..(9): CYP5A1 (Residues 249-257)
DELNGFFNK

SEQ ID NO: 320 - protein, 10 aa, Homo sapiens
  MISC_FEATURE (1)..(10): CYP5A1 (Residues 277-286)
DFLQMVLDAR

SEQ ID NO: 321 - protein, 15 aa, Homo sapiens
  MISC_FEATURE (1)..(15): CYP5A1 (Residues 287-301)
HSASPMGVQDFDIVR

SEQ ID NO: 322 - protein, 14 aa, Homo sapiens
  MISC_FEATURE (1)..(14): CYP5A1 (Residues 302-315)
DVFSSTGCKPNPSR

SEQ ID NO: 323 - protein, 12 aa, Homo sapiens
  MISC_FEATURE (1)..(12): CYP5A1 (Residues 413-424)
EAAQDCEVLGQR

SEQ ID NO: 324 - protein, 16 aa, Homo sapiens
  MISC_FEATURE (1)..(16): CYP5A1 (Residues 462-477)
QQHRPFTYLPFGAGPR

SEQ ID NO: 325 - protein, 9 aa, Homo sapiens
  MISC_FEATURE (1)..(9): CYP5A1 (Residues 491-499)
LTLLHVLHK

SEQ ID NO: 326 - protein, 16 aa, Homo sapiens
  MISC_FEATURE (1)..(16): CYP5A1 (Residues 502-517)
FQACPETQVPLQLESK

SEQ ID NO: 327 - protein, 13 aa, Homo sapiens
  MISC_FEATURE (1)..(13): CYP7A1 (Residues 75-87)
YVHFITNPLSYHK

SEQ ID NO: 328 - protein, 19 aa, Homo sapiens
  MISC_FEATURE (1)..(19): CYP7A1 (Residues 113-131)
SIDPMDGNTTENINDTFIK

SEQ ID NO: 329 - protein, 20 aa, Homo sapiens
  MISC_FEATURE (1)..(20): CYP7A1 (Residues 132-151)
TLQGHALNSLTESMMENLQR

SEQ ID NO: 330 - protein, 11 aa, Homo sapiens
  MISC_FEATURE (1)..(11): CYP7A1 (Residues 152-162)
IMRPPVSSNSK

SEQ ID NO: 331 - protein, 15 aa, Homo sapiens
  MISC_FEATURE (1)..(15): CYP7A1 (Residues 163-177)
TAAWVTEGMYSFCYR

SEQ ID NO: 332 - protein, 13 aa, Homo sapiens
  MISC_FEATURE (1)..(13): CYP7A1 (Residues 178-190)
VMFEAGYLTIFGR

SEQ ID NO: 333 - protein, 11 aa, Homo sapiens
  MISC_FEATURE (1)..(11): CYP7A1 (Residues 200-210)
AHILNNLDNFK

SEQ ID NO: 334 - protein, 15 aa, Homo sapiens
  MISC_FEATURE (1)..(15): CYP7A1 (Residues 215-229)
VFPALVAGLPIHMFR

SEQ ID NO: 335 - protein, 10 aa, Homo sapiens
  MISC_FEATURE (1)..(10): CYP7A1 (Residues 251-260)
ESISELISLR

SEQ ID NO: 336 - protein, 15 aa, Homo sapiens
  MISC_FEATURE (1)..(15): CYP7A1 (Residues 261-275)
MFLNDTLSTFDDLEK

SEQ ID NO: 337 - protein, 9 aa, Homo sapiens
  MISC_FEATURE (1)..(9): CYP7A1 (Residues 356-364)
LSSASLNIR

SEQ ID NO: 338 - protein, 15 aa, Homo sapiens
  MISC_FEATURE (1)..(15): CYP7A1 (Residues 368-382)
EDFTLHLEDGSYNIR

SEQ ID NO: 339 - protein, 9 aa, Homo sapiens
  MISC_FEATURE (1)..(9): CYP7A1 (Residues 421-429)
TTFYCNGLK

SEQ ID NO: 340 - protein, 16 aa, Homo sapiens
  MISC_FEATURE (1)..(16): CYP7A1 (Residues 432-447)
YYYMPFGSGATICPGR

SEQ ID NO: 341 - protein, 15 aa, Homo sapiens
  MISC_FEATURE (1)..(15): CYP7A1 (Residues 484-498)
AGLGILPPLNDIEFK

SEQ ID NO: 342 - protein, 11 aa, Homo sapiens
  MISC_FEATURE (1)..(11): CYP7B1 (Residues 1-11)
MAGEVSAATGR

SEQ ID NO: 343 - protein, 13 aa, Homo sapiens
  MISC_FEATURE (1)..(13): CYP7B1 (Residues 51-63)
GWLPYLGVVLNLR

SEQ ID NO: 344 - protein, 13 aa, Homo sapiens
  MISC_FEATURE (1)..(13): CYP7B1 (Residues 76-88)
QHGDTFTVLLGGK

SEQ ID NO: 345 - protein, 16 aa, Homo sapiens
  MISC_FEATURE (1)..(16): CYP7B1 (Residues 89-104)
YITFILDPFQYQLVIK

SEQ ID NO: 346 - protein, 9 aa, Homo sapiens
  MISC_FEATURE (1)..(9): CYP7B1 (Residues 122-130)
AFSISQLQK

SEQ ID NO: 347 - protein, 14 aa, Homo sapiens
  MISC_FEATURE (1)..(14): CYP7B1 (Residues 149-162)
SLDILLESMMQNLK

SEQ ID NO: 348 - protein, 9 aa, Homo sapiens
  MISC_FEATURE (1)..(9): CYP7B1 (Residues 163-171)
QVFEPQLLK

SEQ ID NO: 349 - protein, 17 aa, Homo sapiens
  MISC_FEATURE (1)..(17): CYP7B1 (Residues 223-239)
FAYLVSNIPIELLGNVK

SEQ ID NO: 350 - protein, 11 aa, Homo sapiens
  MISC_FEATURE (1)..(11): CYP7B1 (Residues 257-267)
MQGWSEVFQSR

SEQ ID NO: 351 - protein, 9 aa, Homo sapiens
  MISC_FEATURE (1)..(9): CYP7B1 (Residues 311-319)
HPEAMAAVR

SEQ ID NO: 352 - protein, 10 aa, Homo sapiens
  MISC_FEATURE (1)..(10): CYP7B1 (Residues 334-343)
GSGFPIHLTR

SEQ ID NO: 353 - protein, 9 aa, Homo sapiens
  MISC_FEATURE (1)..(9): CYP7B1 (Residues 362-370)
LSSYSTTIR

SEQ ID NO: 354 - protein, 18 aa, Homo sapiens
  MISC_FEATURE (1)..(18): CYP7B1 (Residues 371-388)
FVEEDLTLSSETGDYCVR

SEQ ID NO: 355 - protein, 12 aa, Homo sapiens
  MISC_FEATURE (1)..(12): CYP7B1 (Residues 437-448)
CYLMPFGTGTSK

SEQ ID NO: 356 - protein, 15 aa, Homo sapiens
  MISC_FEATURE (1)..(15): CYP7B1 (Residues 487-501)
LLFGIQYPDSDVLFR

SEQ ID NO: 357 - protein, 12 aa, Homo sapiens
  MISC_FEATURE (1)..(12): CYP8A1 (Residues 61-72)
HGDIFTILVGGR

SEQ ID NO: 358 - protein, 13 aa, Homo sapiens
  MISC_FEATURE (1)..(13): CYP8A1 (Residues 94-106)
LDFHAYAIFLMER

SEQ ID NO: 359 - protein, 15 aa, Homo sapiens
  MISC_FEATURE (1)..(15): CYP8A1 (Residues 107-121)
IFDVQLPHYSPSDEK

SEQ ID NO: 360 - protein, 14 aa, Homo sapiens
  MISC_FEATURE (1)..(14): CYP8A1 (Residues 175-188)
AGYLTLYGIEALPR

SEQ ID NO: 361 - protein, 9 aa, Homo sapiens
  MISC_FEATURE (1)..(9): CYP8A1 (Residues 189-197)
THESQAQDR

SEQ ID NO: 362 - protein, 11 aa, Homo sapiens
  MISC_FEATURE (1)..(11): CYP8A1 (Residues 198-208)
VHSADVFHTFR

SEQ ID NO: 363 - protein, 9 aa, Homo sapiens
  MISC_FEATURE (1)..(9): CYP8A1 (Residues 302-310)
NPEALAAVR

SEQ ID NO: 364 - protein, 17 aa, Homo sapiens
  MISC_FEATURE (1)..(17): CYP8A1 (Residues 334-350)
VLDSTPVLDSVLSESLR

SEQ ID NO: 365 - protein, 9 aa, Homo sapiens
  MISC_FEATURE (1)..(9): CYP8A1 (Residues 351-359)
LTAAPFITR

SEQ ID NO: 366 - protein, 14 aa, Homo sapiens
  MISC_FEATURE (1)..(14): CYP8A1 (Residues 360-373)
EVVVDLAMPMADGR

SEQ ID NO: 367 - protein, 11 aa, Homo sapiens
  MISC_FEATURE (1)..(11): CYP8A1 (Residues 383-393)
LLLFPFLSPQR

SEQ ID NO: 368 - protein, 12 aa, Homo sapiens
  MISC_FEATURE (1)..(12): CYP8A1 (Residues 394-405)
DPEIYTDPEVFK

SEQ ID NO: 369 - protein, 9 aa, Homo sapiens
  MISC_FEATURE (1)..(9): CYP8A1 (Residues 409-417)
FLNPDGSEK

SEQ ID NO: 370 - protein, 16 aa, Homo sapiens
  MISC_FEATURE (1)..(16): CYP8A1 (Residues 429-444)
NYNMPWGAGHNHCLGR

SEQ ID NO: 371 - protein, 15 aa, Homo sapiens
  MISC_FEATURE (1)..(15): CYP8A1 (Residues 481-495)
YGFGLMQPEHDVPVR

SEQ ID NO: 372 - protein, 13 aa, Homo sapiens
  MISC_FEATURE (1)..(13): CYP8B1 (Residues 38-50)
GTVPWLGHAMAFR

SEQ ID NO: 373 - protein, 15 aa, Homo sapiens
  MISC_FEATURE (1)..(15): CYP8B1 (Residues 115-129)
SVQGDHEMIHSASTK

SEQ ID NO: 374 - protein, 16 aa, Homo sapiens
  MISC_FEATURE (1)..(16): CYP8B1 (Residues 156-171)
GWSLDASCWHEDSLFR

SEQ ID NO: 375 - protein, 15 aa, Homo sapiens
  MISC_FEATURE (1)..(15): CYP8B1 (Residues 193-207)
EQDLLQAGELFMEFR

SEQ ID NO: 376 - protein, 9 aa, Homo sapiens
  MISC_FEATURE (1)..(9): CYP8B1 (Residues 216-224)
FVYSLLWPR

SEQ ID NO: 377 - protein, 10 aa, Homo sapiens
  MISC_FEATURE (1)..(10): CYP8B1 (Residues 239-248)
MLSVSHSQEK

SEQ ID NO: 378 - protein, 15 aa, Homo sapiens
  MISC_FEATURE (1)..(15): CYP8B1 (Residues 249-263)
EGISNWLGNMLQFLR

SEQ ID NO: 379 - protein, 11 aa, Homo sapiens
  MISC_FEATURE (1)..(11): CYP8B1 (Residues 264-274)
EQGVPSAMQDK

SEQ ID NO: 380 - protein, 11 aa, Homo sapiens
  MISC_FEATURE (1)..(11): CYP8B1 (Residues 310-320)
EEATQVLGEAR

SEQ ID NO: 381 - protein, 19 aa, Homo sapiens
  MISC_FEATURE (1)..(19): CYP8B1 (Residues 331-349)
LGALQHTPVLDSVVEETLR

SEQ ID NO: 382 - protein, 9 aa, Homo sapiens
  MISC_FEATURE (1)..(9): CYP8B1 (Residues 359-367)
LVHEDTYLK

SEQ ID NO: 383 - protein, 10 aa, Homo sapiens
  MISC_FEATURE (1)..(10): CYP8B1 (Residues 368-377)
MSSGQEYLFR

SEQ ID NO: 384 - protein, 18 aa, Homo sapiens
  MISC_FEATURE (1)..(18): CYP8B1 (Residues 426-443)
IHHYTMPWGSGVSICPGR

SEQ ID NO: 385 - protein, 13 aa, Homo sapiens
  MISC_FEATURE (1)..(13): CYP8B1 (Residues 480-492)
WGFGTMQPSHDVR

SEQ ID NO: 386 - protein, 10 aa, Homo sapiens
  MISC_FEATURE (1)..(10): CYP11A1 (Residues 15-24)
GYQTFLSAPR

SEQ ID NO: 387 - protein, 12 aa, Homo sapiens
  MISC_FEATURE (1)..(12): CYP11A1 (Residues 32-43)
VPTGEGAGISTR

SEQ ID NO: 388 - protein, 11 aa, Homo sapiens
  MISC_FEATURE (1)..(11): CYP11A1 (Residues 74-84)
VHLHHVQNFQK

SEQ ID NO: 389 - protein, 14 aa, Homo sapiens
  MISC_FEATURE (1)..(14): CYP11A1 (Residues 152-165)
VALNQEVMAPEATK

SEQ ID NO: 390 - protein, 11 aa, Homo sapiens
  MISC_FEATURE (1)..(11): CYP11A1 (Residues 166-176)
NFLPLLDAVSR

SEQ ID NO: 391 - protein, 16 aa, Homo sapiens
  MISC_FEATURE (1)..(16): CYP11A1 (Residues 189-204)
AGSGNYSGDISDDLFR

SEQ ID NO: 392 - protein, 14 aa, Homo sapiens
  MISC_FEATURE (1)..(14): CYP11A1 (Residues 205-218)
FAFESITNVIFGER

SEQ ID NO: 393 - protein, 14 aa, Homo sapiens
  MISC_FEATURE (1)..(14): CYP11A1 (Residues 219-232)
QGMLEEVVNPEAQR

SEQ ID NO: 394 - protein, 12 aa, Homo sapiens
  MISC_FEATURE (1)..(12): CYP11A1 (Residues 265-276)
DHVAAWDVIFSK

SEQ ID NO: 395 - protein, 13 aa, Homo sapiens
  MISC_FEATURE (1)..(13): CYP11A1 (Residues 277-289)
ADIYTQNFYWELR

SEQ ID NO: 396 - protein, 18 aa, Homo sapiens
  MISC_FEATURE (1)..(18): CYP11A1 (Residues 361-378)
HQAQGDMATMLQLVPLLK

SEQ ID NO: 397 - protein, 10 aa, Homo sapiens
  MISC_FEATURE (1)..(10): CYP11A1 (Residue 387-396)
LHPISVTLQR

SEQ ID NO: 398 - protein, 9 aa, Homo sapiens
  MISC_FEATURE (1)..(9): CYP11A1 (Residues 397-405)
YLVNDLVLR

SEQ ID NO: 399 - protein, 12 aa, Homo sapiens
  MISC_FEATURE (1)..(12): CYP11A1 (Residues 413-424)
TLVQVAIYALGR

SEQ ID NO: 400 - protein, 15 aa, Homo sapiens
  MISC_FEATURE (1)..(15): CYP11A1 (Residues 425-439)
EPTFFFDPENFDPTR

SEQ ID NO: 401 - protein, 9 aa, Homo sapiens
  MISC_FEATURE (1)..(9): CYP11A1 (Residues 452-460)
NLGFGWGVR

SEQ ID NO: 402 - protein, 14 aa, Homo sapiens
  MISC_FEATURE (1)..(14): CYP11B1 (Residues 7-20)
AEVCMAVPWLSLQR

SEQ ID NO: 403 - protein, 11 aa, Homo sapiens
  MISC_FEATURE (1)..(11): CYP11B1 (Residues 100-110)
LQQVDSLHPHR

SEQ ID NO: 404 - protein, 10 aa, Homo sapiens
  MISC_FEATURE (1)..(10): CYP11B1 (Residues 111-120)
MSLEPWVAYR

SEQ ID NO: 405 - protein, 13 aa, Homo sapiens
  MISC_FEATURE (1)..(13): CYP11B1 (Residues 144-156)
LNPEVLSPNAVQR

SEQ ID NO: 406 - protein, 9 aa, Homo sapiens
  MISC_FEATURE (1)..(9): CYP11B1 (Residues 333-341)
NPNVQQALR

SEQ ID NO: 407 - protein, 12 aa, Homo sapiens
  MISC_FEATURE (1)..(12): CYP11B1 (Residues 437-448)
NFYHVPFGFGMR

SEQ ID NO: 408 - protein, 15 aa, Homo sapiens
  MISC_FEATURE (1)..(15): CYP11B1 (Residues 455-469)
LAEAEMLLLLHHVLK

SEQ ID NO: 409 - protein, 13 aa, Homo sapiens
  MISC_FEATURE (1)..(13): CYP11B1 (Residues 470-482)
HLQVETLTQEDIK

SEQ ID NO: 410 - protein, 14 aa, Homo sapiens
  MISC_FEATURE (1)..(14): CYP11B2 (Residues 7-20)
AEVCVAAPWLSLQR

SEQ ID NO: 411 - protein, 15 aa, Homo sapiens
  MISC_FEATURE (1)..(15): CYP11B2 (Residues 34-48)
TVLPFEAMPQHPGNR

SEQ ID NO: 412 - protein, 12 aa, Homo sapiens
  MISC_FEATURE (1)..(12): CYP11B2 (Residues 88-99)
MVCVMLPEDVEK

SEQ ID NO: 413 - protein, 11 aa, Homo sapiens
  MISC_FEATURE (1)..(11): CYP11B2 (Residues 100-110)
LQQVDSLHPCR

SEQ ID NO: 414 - protein, 10 aa, Homo sapiens
  MISC_FEATURE (1)..(10): CYP11B2 (Residues 111-120)
MILEPWVAYR

SEQ ID NO: 415 - protein, 9 aa, Homo sapiens
  MISC_FEATURE (1)..(9): CYP11B2 (Residues 333-341)
NPDVQQILR

SEQ ID NO: 416 - protein, 10 aa, Homo sapiens
  MISC_FEATURE (1)..(10): CYP11B2 (Residues 413-422)
NAALFPRPER

SEQ ID NO: 417 - protein, 12 aa, Homo sapiens
  MISC_FEATURE (1)..(12): CYP11B2 (Residues 437-448)
NFHHVPFGFGMR

SEQ ID NO: 418 - protein, 15 aa, Homo sapiens
  MISC_FEATURE (1)..(15): CYP11B2 (Residues 470-482)
LAEAEMLLLLHHVLK

SEQ ID NO: 419 - protein, 16 aa, Homo sapiens
  MISC_FEATURE (1)..(16): CYP17A1 (Residues 30-45)
SLLSLPLVGSLPFLPR

SEQ ID NO: 420 - protein, 10 aa, Homo sapiens
  MISC_FEATURE (1)..(10): CYP17A1 (Residues 46-55)
HGHMHNNFFK

SEQ ID NO: 421 - protein, 12 aa, Homo sapiens
  MISC_FEATURE (1)..(12): CYP17A1 (Residues 72-83)
TTVIVGHHQLAK

SEQ ID NO: 422 - protein, 18 aa, Homo sapiens
  MISC_FEATURE (1)..(18): CYP17A1 (Residues 92-109)
DFSGRPQMATLDIASNNR

SEQ ID NO: 423 - protein, 15 aa, Homo sapiens
  MISC_FEATURE (1)..(15): CYP17A1 (Residues 111-125)
GIAFADSGAHWQLHR

SEQ ID NO: 424 - protein, 10 aa, Homo sapiens
  MISC_FEATURE (1)..(10): CYP17A1 (Residues 127-136)
LAMATFALFK

SEQ ID NO: 425 - protein, 11 aa, Homo sapiens
  MISC_FEATURE (1)..(11): CYP17A1 (Residues 212-222)
DSLVDLVPWLK

SEQ ID NO: 426 - protein, 15 aa, Homo sapiens
  MISC_FEATURE (1)..(15): CYP17A1 (Residues 256-270)
SDSITNMLDTLMQAK

SEQ ID NO: 427 - protein, 13 aa, Homo sapiens
  MISC_FEATURE (1)..(13): CYP17A1 (Residues 313-325)
WTLAFLLHNPQVK

SEQ ID NO: 428 - protein, 13 aa, Homo sapiens
  MISC_FEATURE (1)..(13): CYP17A1 (Residues 328-340)
LYEEIDQNVGFSR

SEQ ID NO: 429 - protein, 9 aa, Homo sapiens
  MISC_FEATURE (1)..(9): CYP17A1 (Residues 350-358)
LLLLEATIR

SEQ ID NO: 430 - protein, 14 aa, Homo sapiens
  MISC_FEATURE (1)..(14): CYP17A1 (Residues 375-388)
ANVDSSIGEFAVDK

SEQ ID NO: 431 - protein, 16 aa, Homo sapiens
  MISC_FEATURE (1)..(16): CYP17A1 (Residues 389-404)
GTEVIINLWALHHNEK

SEQ ID NO: 432 - protein, 12 aa, Homo sapiens
  MISC_FEATURE (1)..(12): CYP17A1 (Residues 405-416)
EWHQPDQFMPER

SEQ ID NO: 433 - protein, 13 aa, Homo sapiens
  MISC_FEATURE (1)..(13): CYP17A1 (Residues 450-462)
QELFLIMAWLLQR

SEQ ID NO: 434 - protein, 9 aa, Homo sapiens
  MISC_FEATURE (1)..(9): CYP17A1 (Residue 482-490)
VVFLIDSFK

SEQ ID NO: 435 - protein, 15 aa, Homo sapiens
  MISC_FEATURE (1)..(15): CYP19A1 (Residues 64-78)
FLWMGIGSACNYYNR

SEQ ID NO: 436 - protein, 13 aa, Homo sapiens
  MISC_FEATURE (1)..(13): CYP19A1 (Residues 86-98)
VWISGEETLIISK

SEQ ID NO: 437 - protein, 9 aa, Homo sapiens
  MISC_FEATURE (1)..(9): CYP19A1 (Residues 99-107)
SSSMFHIMK

SEQ ID NO: 438 - protein, 11 aa, Homo sapiens
  MISC_FEATURE (1)..(11): CYP19A1 (Residues 119-129)
LGLQCIGMHEK

SEQ ID NO: 439 - protein, 12 aa, Homo sapiens
  MISC_FEATURE (1)..(12): CYP19A1 (Residues 130-141)
GIIFNNNPELWK

SEQ ID NO: 440 - protein, 10 aa, Homo sapiens
  MISC_FEATURE (1)..(10): CYP19A1 (Residues 159-168)
MVTVCAESLK

SEQ ID NO: 441 - protein, 12 aa, Homo sapiens
  MISC_FEATURE (1)..(12): CYP19A1 (Residues 193-204)
VMLDTSNTLFLR

SEQ ID NO: 442 - protein, 11 aa, Homo sapiens
  MISC_FEATURE (1)..(11): CYP19A1 (Residues 205-215)
IPLDESAIVVK

SEQ ID NO: 443 - protein, 10 aa, Homo sapiens
  MISC_FEATURE (1)..(10): CYP19A1 (Residues 252-261)
DAIEVLIAEK

SEQ ID NO: 444 - protein, 16 aa, Homo sapiens
  MISC_FEATURE (1)..(16): CYP19A1 (Residues 271-286)
LEECMDFATELILAEK

SEQ ID NO: 445 - protein, 10 aa, Homo sapiens
  MISC_FEATURE (1)..(10): CYP19A1 (Residues 324-333)
HPNVEEAIIK

SEQ ID NO: 446 - protein, 9 aa, Homo sapiens
  MISC_FEATURE (1)..(9): CYP19A1 (Residues 334-342)
EIQTVIGER

SEQ ID NO: 447 - protein, 11 aa, Homo sapiens
  MISC_FEATURE (1)..(11): CYP19A1 (Residues 354-364)
VMENFIYESMR

SEQ ID NO: 448 - protein, 10 aa, Homo sapiens
  MISC_FEATURE (1)..(10): CYP19A1 (Residues 365-374)
YQPVVDLVMR

SEQ ID NO: 449 - protein, 13 aa, Homo sapiens
  MISC_FEATURE (1)..(13): CYP19A1 (Residues 376-388)
ALEDDVIDGYPVK

SEQ ID NO: 450 - protein, 10 aa, Homo sapiens
  MISC_FEATURE (1)..(10): CYP19A1 (Residues 390-399)
GTNIILNIGR

SEQ ID NO: 451 - protein, 10 aa, Homo sapiens
  MISC_FEATURE (1)..(10): CYP19A1 (Residues 425-434)
YFQPFGFGPR

SEQ ID NO: 452 - protein, 12 aa, Homo sapiens
  MISC_FEATURE (1)..(12): CYP19A1 (Residues 461-472)
TLQGQCVESIQK

SEQ ID NO: 453 - protein, 12 aa, Homo sapiens
  MISC_FEATURE (1)..(12): CYP19A1 (Residues 473-484)
IHDLSLHPDETK

SEQ ID NO: 454 - protein, 10 aa, Homo sapiens
  MISC_FEATURE (1)..(10): CYP19A1 (Residues 485-494)
NMLEMIFTPR

SEQ ID NO: 455 - protein, 14 aa, Homo sapiens
  MISC_FEATURE (1)..(14): CYP39A1 (Residues 34-47)
GWIPWIGVGFEFGK

SEQ ID NO: 456 - protein, 13 aa, Homo sapiens
  MISC_FEATURE (1)..(13): CYP39A1 (Residues 60-72)
YGPIFTVFAMGNR

SEQ ID NO: 457 - protein, 15 aa, Homo sapiens
  MISC_FEATURE (1)..(15): CYP39A1 (Residues 73-87)
MTFVTEEEGINVFLK

SEQ ID NO: 458 - protein, 13 aa, Homo sapiens
  MISC_FEATURE (1)..(13): CYP39A1 (Residues 91-103)
VDFELAVQNIVYR

SEQ ID NO: 459 - protein, 9 aa, Homo sapiens
  MISC_FEATURE (1)..(9): CYP39A1 (Residues 110-118)
NVFLALHEK

SEQ ID NO: 460 - protein, 15 aa, Homo sapiens
  MISC_FEATURE (1)..(15): CYP39A1 (Residues 164-177)
HLLYPVTVNYMLFNK

SEQ ID NO: 461 - protein, 12 aa, Homo sapiens
  MISC_FEATURE (1)..(12): CYP39A1 (Residues 298-309)
AIMEGISSVFGK

SEQ ID NO: 462 - protein, 12 aa, Homo sapiens
  MISC_FEATURE (1)..(12): CYP39A1 (Residues 378-389)
YFPEPELFKPER

SEQ ID NO: 463 - protein, 14 aa, Homo sapiens
  MISC_FEATURE (1)..(14): CYP39A1 (Residues 398-411)
HSFLDCFMAFGSGK

SEQ ID NO: 464 - protein, 11 aa, Homo sapiens
  MISC_FEATURE (1)..(11): CYP39A1 (Residues 435-445)
YDCSLLDPLPK

SEQ ID NO: 465 - protein, 17 aa, Homo sapiens
  MISC_FEATURE (1)..(17): CYP39A1 (Residues 446-462)
QSYLHLVGVPQPEGQCR

SEQ ID NO: 466 - protein, 11 aa, Homo sapiens
  MISC_FEATURE (1)..(11): CYP46A1 (Residues 59-69)
VLQDVFLDWAK

SEQ ID NO: 467 - protein, 12 aa, Homo sapiens
  MISC_FEATURE (1)..(12): CYP46A1 (Residues 83-94)
TSVIVTSPESVK

SEQ ID NO: 468 - protein, 9 aa, Homo sapiens
  MISC_FEATURE (1)..(9): CYP46A1 (Residues 111-119)
ALQTVFGER

SEQ ID NO: 469 - protein, 14 aa, Homo sapiens
  MISC_FEATURE (1)..(14): CYP46A1 (Residues 120-133)
LFGQGLVSECNYER

SEQ ID NO: 470 - protein, 13 aa, Homo sapiens
  MISC_FEATURE (1)..(13): CYP46A1 (Residues 148-160)
SSLVSLMETFNEK

SEQ ID NO: 471 - protein, 11 aa, Homo sapiens
  MISC_FEATURE (1)..(11): CYP46A1 (Residues 161-171)
AEQLVEILEAK

SEQ ID NO: 472 - protein, 10 aa, Homo sapiens
  MISC_FEATURE (1)..(10): CYP46A1 (Residues 217-226)
LMLEGITASR

SEQ ID NO: 473 - protein, 14 aa, Homo sapiens
  MISC_FEATURE (1)..(14): CYP46A1 (Residues 268-281)
GEEVPADILTQILK

SEQ ID NO: 474 - protein, 12 aa, Homo sapiens
  MISC_FEATURE (1)..(12): CYP46A1 (Residues 328-339)
LQAEVDEVIGSK

SEQ ID NO: 475 - protein, 9 aa, Homo sapiens
  MISC_FEATURE (1)..(9): CYP46A1 (Residues 341-349)
YLDFEDLGR

SEQ ID NO: 476 - protein, 9 aa, Homo sapiens
  MISC_FEATURE (1)..(9): CYP46A1 (Residues 350-358)
LQYLSQVLK

SEQ ID NO: 477 - protein, 10 aa, Homo sapiens
  MISC_FEATURE (1)..(10): CYP46A1 (Residues 363-372)
LYPPAWGTFR

SEQ ID NO: 478 - protein, 12 aa, Homo sapiens
  MISC_FEATURE (1)..(12): CYP46A1 (Residues 373-384)
LLEEETLIDGVR

SEQ ID NO: 479 - protein, 16 aa, Homo sapiens
  MISC_FEATURE (1)..(16): CYP46A1 (Residues 385-400)
VPGNTPLLFSTYVMGR

SEQ ID NO: 480 - protein, 15 aa, Homo sapiens
  MISC_FEATURE (1)..(15): CYP46A1 (Residues 401-415)
MDTYFEDPLTFNPDR

SEQ ID NO: 481 - protein, 11 aa, Homo sapiens
  MISC_FEATURE (1)..(11): CYP46A1 (Residues 425-435)
FTYFPFSLGHR

SEQ ID NO: 482 - protein, 13 aa, Homo sapiens
  MISC_FEATURE (1)..(13): CYP46A1 (Residues 436-448)
SCIGQQFAQMEVK

SEQ ID NO: 483 - protein, 14 aa, Homo sapiens
  MISC_FEATURE (1)..(14): CYP51A1 (Residues 46-59)
LAAGHLVQLPAGVK

SEQ ID NO: 484 - protein, 12 aa, Homo sapiens
  MISC_FEATURE (1)..(12): CYP51A1 (Residues 80-91)
SPIEFLENAYEK

SEQ ID NO: 485 - protein, 12 aa, Homo sapiens
  MISC_FEATURE (1)..(12): CYP51A1 (Residues 92-103)
YGPVFSFTMVGK

SEQ ID NO: 486 - protein, 12 aa, Homo sapiens
  MISC_FEATURE (1)..(12): CYP51A1 (Residues 122-133)
NEDLNAEDVYSR

SEQ ID NO: 487 - protein, 15 aa, Homo sapiens
  MISC_FEATURE (1)..(15): CYP51A1 (Residues 142-156)
GVAYDVPNPVFLEQK

SEQ ID NO: 488 - protein, 12 aa, Homo sapiens
  MISC_FEATURE (1)..(12): CYP51A1 (Residues 181-192)
EYFESWGESGEK

SEQ ID NO: 489 - protein, 14 aa, Homo sapiens
  MISC_FEATURE (1)..(14): CYP51A1 (Residues 278-291)
IDDILQTLLDATYK

SEQ ID NO: 490 - protein, 16 aa, Homo sapiens
  MISC_FEATURE (1)..(16): CYP51A1 (Residues 343-358)
TVCGENLPPLTYDQLK

SEQ ID NO: 491 - protein, 10 aa, Homo sapiens
  MISC_FEATURE (1)..(10): CYP51A1 (Residues 373-382)
LRPPIMIMMR

SEQ ID NO: 492 - protein, 11 aa, Homo sapiens
  MISC_FEATURE (1)..(11): CYP51A1 (Residues 426-436)
YLQDNPASGEK

SEQ ID NO: 493 - protein, 10 aa, Homo sapiens
  MISC_FEATURE (1)..(10): CYP51A1 (Residues 437-446)
FAYVPFGAGR

SEQ ID NO: 494 - protein, 12 aa, Homo sapiens
  MISC_FEATURE (1)..(12): CYP51A1 (Residues 449-460)
CIGENFAYVQIK

SEQ ID NO: 495 - protein, 21 aa, Homo sapiens
  MISC_FEATURE (1)..(21): CYP2B1 (Residues 1-21)
MEPTILLLLALLVGFLLLLVR

SEQ ID NO: 496 - protein, 21 aa, Homo sapiens
  MISC_FEATURE (1)..(21): CYP2B2 (Residues 1-21)
MEPSILLLLALLVGFLLLLVR

SEQ ID NO: 497 - protein, 7 aa, Homo sapiens
  MISC_FEATURE (1)..(7): CYP2B1 (Residues 317-323)
YPHVAEK

SEQ ID NO: 498 - protein, 7 aa, Homo sapiens
  MISC_FEATURE (1)..(7): CYP2B2 (Residues 317-323)
YPHVTEK

SEQ ID NO: 499 - protein, 17 aa, Homo sapiens
  MISC_FEATURE (1)..(17): CYP2B1 (Residues 327-336)
EIDQVIGSHRLPTLDDR

SEQ ID NO: 500 - protein, 17 aa, Homo sapiens
  MISC_FEATURE (1)..(17): CYP2B2 (Residues 327-343)
EIDQVIGSHRPPSLDDR

SEQ ID NO: 501 - protein, 12 aa, Homo sapiens
  MISC_FEATURE (1)..(12): CYP2B1 (Residues 359-370)
FSDLVPIGVPHR

SEQ ID NO: 502 - protein, 12 aa, Homo sapiens
  MISC_FEATURE (1)..(12): CYP2B2 (Residues 359-370)
FADLAPIGLPHR